(19) United States
(12) Patent Application Publication
(10) Pub. No.: US 2023/0085044 A1
(43) Pub. Date: Mar. 16, 2023

(54) METHODS, SYSTEMS, AND APPARATUS FOR PROBABILISTIC REASONING

(71) Applicant: Minerva Intelligence Inc., Vancouver (CA)

(72) Inventors: David Lynton Poole, Vancouver (CA); Clinton Paul Smyth, Vancouver (CA)

(73) Assignee: Minerva Intelligence Inc., Vancouver, BC (CA)

(21) Appl. No.: 17/800,355

(22) PCT Filed: Feb. 19, 2021
(86) PCT No.: PCT/CA2021/050189

§ 371 Date: Aug. 17, 2022

(30) Foreign Application Priority Data
Feb. 19, 2020 (CA) 3072901
Feb. 18, 2021 (CA) 3109301

(51) Int. Cl. G06N 7/00; G06F 40/30

(52) U.S. Cl. CPC G06N 7/005; G06F 40/30

(57) ABSTRACT

A device may be provided for expressing a probabilistic reasoning of an attribute in a conceptual model. A model attribute may be determined that may be relevant for a model. The model may be determined by expressing at least two of a frequency of the model attribute in the model, a frequency of the model attribute in a default model, a probabilistic reasoning of a presence of the model attribute, a probabilistic reasoning of an absence of the model attribute. An instance may be determined and may include at least an instance attribute that has a positive probabilistic reasoning or a negative probabilistic reasoning. A predictive score may be determined for the instance using a contribution made by the instance attribute. An explanation associated with the predictive score may be determined.

### BACKGROUND

The rise of artificial intelligence (AI) may be one of the most significant trends in the technology sector over the coming years. Advances in AI may impact companies of all sizes and in various sectors as businesses look to improve decision-making, reduce operating costs and enhance consumer experience. The concept of what defines AI has changed over time, but at its core are machines being able to perform tasks that may require human perception or cognition.

Recent breakthroughs in AI have been achieved by applying machine learning to very large data sets. However, machine learning has limitations in that machine learning often may fail when there may be limited training data available or when the actual dataset differs from the training set. Also, it is often difficult to get clear explanations of the results produced by deep learning systems.

### SUMMARY OF THE INVENTION

Disclosed herein are systems, methods, and apparatus that provide probabilistic reasoning to generate predictive analyses. Probabilistic reasoning may assist machine learning where there may be limited training data available or when the dataset differs from the training set. Probabilistic reasoning may also provide explanations of the results produced by deep learning systems.

Probabilistic reasoning may use human generated knowledge models to generate predictive analyses. For example, semantic networks may be used as a data format, which may allow for explanations to be provided in a natural language. Probabilistic reasoning may provide predictions and may provide advice (e.g., expert advice).

As disclosed herein, artificial intelligence may be used to provide a probabilistic interpretation of scores. For example, the artificial intelligence may provide probabilistic reasoning with (e.g., using) complex human-generated and sensed observations. A score used for probabilistic interpretation may be a log base 10 of a probability ratio. For example, scores in a model may be log base 10 of a probability ratio (e.g., similar to the use of logs in decibels or the Richter scale), which provides an order-of-magnitude interpretation to the scores. Whereas the probabilities in a conjunction may be multiplied, the scores may be added.

A score used for probabilistic interpretation may be a measure of surprise; so that a model that makes a prediction (e.g., a surprising prediction) may get a reward for the prediction, but may not get much of a reward for making a prediction that would be expected (e.g., would normally be expected) to be true. For example, a prediction that is usual and/or rate may or may not be unexpected or surprising, and a score may be designed to reflect that. A surprise or unexpected prediction may be relative to a normal. For example, in probability, the normal may be an average, but it may be some other well-defined default, which may alleviate a need for determining the average.

A model with attributes may be used to provide probabilistic interpretation of scores. One or more values or numbers may be specified for an attribute. For example, two numbers may be specified for an attribute (e.g., each attribute) in a model; one number may be applied when the attribute is present in an instance of the model, and the other number may be when the attribute is absent. The rewards may be added to get a score (e.g., total score). In many cases, one of these may be small enough so that it may be effectively ignored, except for cases where it may be the differentiating attribute (in which case it may be a small e value such as 0.001). If the model does not make a prediction about an attribute, that attribute may be ignored.

To provide probabilistic interpretation; of scores, semantics and scores may be used. For example, a semantics for the rewards and scores may provide a principled way to judge correctness and to learn the weights from statistics of the world.

A device for expressing a diagnosticity of an attribute in a conceptual model may be provided. The device may comprise a memory and a processor. The processor may be configured to perform a number of actions. One or more terminologies in a domain of expertise for expressing one or more attributes may be determined. An ontology may be determined using the one or more terminologies in the domain of expertise. A constrained model and a constrained instance may be determined by constraining a model and an instance using the ontology. A calibrated model may be determined by calibrating the constrained model to a default model using a terminology from the one or more terminologies to express a first reward and a second reward. A degree of match between the constrained instance and the calibrated model may be determined.

A method implemented in a device for expressing a diagnosticity of an attribute in a conceptual model may be provided. One or more terminologies in a domain of expertise for expressing one or more attributes may be determined. An ontology may be determined using the one or more terminologies in the domain of expertise. A constrained model and a constrained instance may be determined by constraining a model and an instance using the ontology. A calibrated model may be determined by calibrating the constrained model to a default model using a terminology from the one or more terminologies to express a first reward and a second reward. A degree of match may be determined between the constrained instance and the calibrated model.

A computer readable medium having computer executable instructions stored therein may be provided. The computer executable instructions may comprise a number of actions. For example, one or more terminologies in a domain of expertise for expressing one or more attributes may be determined. An ontology may be determined using the one or more terminologies in the domain of expertise. A constrained model and a constrained instance may be determined by constraining a model and an instance using the ontology. A calibrated model may be determined by calibrating the constrained model to a default model using a terminology from the one or more terminologies to express a first reward and a second reward. A degree of match may be determined between the constrained instance and the calibrated model.

As disclosed herein, a device may be provided for expressing a diagnosticity of an attribute in a conceptual model. The device may include a memory, and a processor, the processor configured to perform a number of actions. One or more model attributes may be determined that may be relevant for a model. The model may be defined by expressing, for each model attribute in the one or more model attributes, at least two of a frequency of the model attribute in the model, a frequency of the model attribute in a default model, a diagnosticity of a presence of the model attribute, and a diagnosticity of an absence of the model attribute. An instance may be determined that may include one or more instance attributes, where an instance attribute in the one or more instance attributes may be assigned a positive diagnosticity when the instance attribute may be present and may be assigned a negative diagnosticity when the instance attribute may be absent. A predictive score for the instance may be determined by summing contributions made by the one or more instance attributes. An explanation associated with the predictive score may be determined for each model attribute in the one or more model attributes using the frequency of the model attribute in the model and the frequency of the model attribute in the default model.

As described herein, a device may be provided for expressing a probabilistic reasoning of an attribute in a conceptual model. The device may include a memory and a processor. The processor may be configured to perform a number of actions. A model attribute may be determined that may be relevant for a model. The model may be determined by expressing at least two of a frequency of the model attribute in the model, a frequency of the model attribute in a default model, a probabilistic reasoning of a presence of the model attribute, a probabilistic reasoning of an absence of the model attribute. An instance may be determined and may include at least an instance attribute that has a positive probabilistic reasoning or a negative probabilistic reasoning. A predictive score may be determined for the instance using a contribution made by the instance attribute. An explanation associated with the predictive score may be determined using the frequency of the model attribute in the model and the frequency of the model attribute in the default model.

As described herein, a method may be provided for expressing a probabilistic reasoning of an attribute in a conceptual model. The method may be performed by a device. A model attribute may be determined that may be relevant for a model. The model may be determined by expressing at least two of a frequency of the model attribute in the model, a frequency of the model attribute in a default model, a probabilistic reasoning of a presence of the model attribute, a probabilistic reasoning of an absence of the model attribute. An instance may be determined and may include at least an instance attribute that has a positive probabilistic reasoning or a negative probabilistic reasoning. A predictive score may be determined for the instance using a contribution made by the instance attribute. An explanation associated with the predictive score may be determined using the frequency of the model attribute in the model and the frequency of the model attribute in the default model.

This Summary is provided to introduce a selection of concepts in a simplified form that are further described herein in the Detailed Description. This Summary is not intended to identify key features or essential features of the claimed subject matter, nor is it intended to be used to limit the scope of the claimed subject matter. Other features are described herein.

### BRIEF DESCRIPTION OF THE DRAWINGS

The Summary and the Detailed Description may be better understood when read in conjunction with the accompanying exemplary drawings. It is understood that the potential embodiments of the disclosed systems and implementations are not limited to those depicted.

FIG. 1 shows an example computing environment that may be used for probabilistic reasoning.

FIG. 2 shows an example of joint probability generated by probabilistic reasoning.

FIG. 3 shows an example depiction of a probability of an attribute in part of a model.

FIG. 4 shows another example depiction of a probability of an attribute in part of a model.

FIG. 5 shows another example depiction of a probability of an attribute in part of a model.

FIG. 6 shows an example depiction of a probability of an attribute that may be rare for a model and may be rare in the background.

FIG. 7 shows an example depiction of a probability of an attribute that may be rare in the background and may not be rare in a model.

FIG. 8 shows an example depiction of a probability of an attribute that may be common in the background.

FIG. 9 shows an example depiction of a probability of an attribute, where the presence of the attribute may indicate a weak positive and an absence of the attribute may indicate a weak negative.

FIG. 10 shows an example depiction of a probability of an attribute, where the presence of the attribute may indicate a weak positive and an absence of the attribute may indicate a weak negative.

FIG. 11 shows an example depiction of a probability of an attribute, where the presence of the attribute may indicate a strong positive and an absence of the attribute may indicate a weak negative.

FIG. 12 shows an example depiction of a probability of an attribute, where the presence of the attribute may indicate a weak positive and an absence of the attribute may indicate a weak negative.

FIG. 13 shows an example depiction of a probability of an attribute, where the presence of the attribute may indicate a weak positive and an absence of the attribute may indicate a weak negative.

FIG. 14A shows an example depiction of default that may be used for interval reasoning.

FIG. 14B shows an example depiction of a model that may be used for interval reasoning.

FIG. 15 shows an example depiction of a density function for one or more of the embodiments.

FIG. 16 shows another example depiction of a density function for one or more of the embodiments.

FIG. 17 shows an example depiction of a model and default for an example slope range.

FIG. 18A depicts an example ontology for a room.

FIG. 18B depicts an example ontology for a household item.

FIG. 18C depicts an example ontology for a wall style.

FIG. 19 depicts an example instance of a model apartment that may use one or more ontologies.

FIG. 20 depicts an example default or background for a room.

FIG. 21 depicts how an example model may differ from a default.

FIG. 22 depicts an example flow chart of a process for expressing a diagnosticity of an attribute in a conceptual model.

FIG. 23 depicts another example flow chart of a process for expressing a diagnosticity of an attribute in a conceptual model.

### DETAILED DESCRIPTION

A detailed description of illustrative embodiments will now be described with reference to the various Figures. Although this description provides a detailed example of possible implementations, it should be noted that the details are intended to be exemplary and in no way limit the scope of the application.

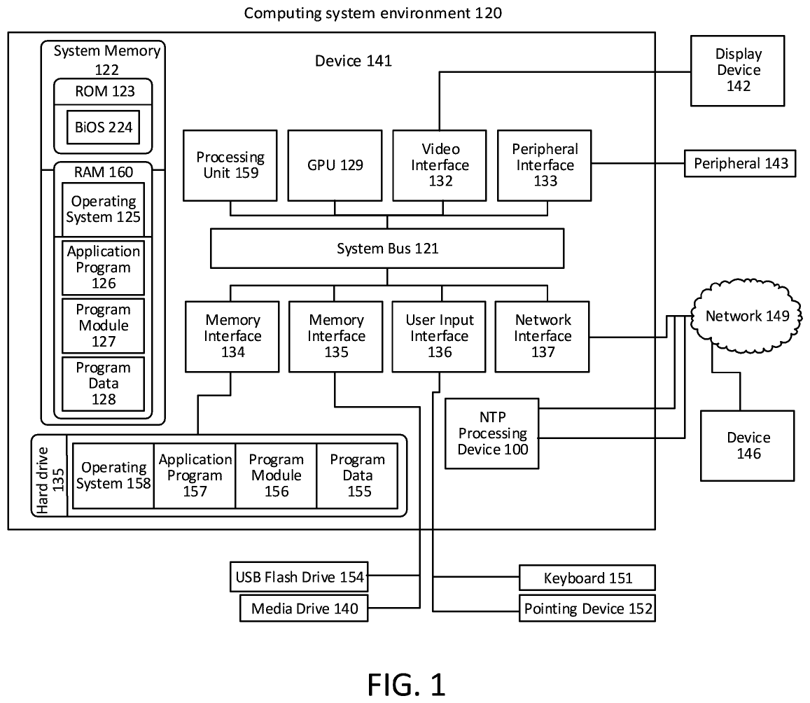

FIG. 1 shows an example computing environment that may be used for probabilistic reasoning. Computing system environment 120 is not intended to suggest any limitation as to the scope of use or functionality of the disclosed subject matter. Computing environment 120 should not be interpreted as having any dependency or requirement relating to the components illustrated in FIG. 1. For example, in some cases, a software process may be transformed into an equivalent hardware structure, and a hardware structure may be transformed into an equivalent software process. The selection of a hardware implementation versus a software implementation may be one of design choice and may be left to the implementer.

The computing elements shown in FIG. 1 may include circuitry that may be configured to implement aspects of the disclosure. The circuitry may include hardware components that may be configured to perform one or more function(s) by firmware or switches. The circuitry may include a processor, a memory, and/or the like, which may be configured by software instructions. The circuitry may include a combination of hardware and software. For example, source code that may embody logic may be compiled into machine-readable code and may be processed by a processor.

As shown in FIG. 1, computing environment 120 may include device 141, which may be a computer, and may include a variety of computer readable media that may be accessed by device 141. Device 141 may be a computer, a cell phone, a server, a database, a tablet, a smart phone, and/or the like. The computer readable media may include volatile media, nonvolatile media, removable media, non-removable media, and/or the like. System memory 122 may include read only memory (ROM) 123 and random access memory (RAM) 160. ROM 123 may include basic input/output system (BIOS) 124. BIOS 124 may include basic routines that may help to transfer data between elements within device 141 during start-up. RAM 160 may include data and/or program modules that may be accessible to by processing unit 159. ROM 123 may include operating system 125, application program 126, program module 127, and program data 128.

Device 141 may also include other computer storage media. For example, device 141 may include hard drive 138, media drive 140, USB flash drive 154, and/or the like. Media drive 140 may be a DVD/CD drive, hard drive, a disk drive, a removable media drive, flash memory cards, digital versatile disks, digital video tape, solid state RAM, solid state ROM, and/or the like. The media drive 140 may be internal or external to device 141. Device 141 may access data on media drive 140 for execution, playback, and/or the like. Hard drive 138 may be connected to system bus 121 by a memory interface such as memory interface 134. Universal serial bus (USB) flash drive 154 and media drive 140 may be connected to the system bus 121 by memory interface 135.

As shown in FIG. 1, the drives and their computer storage media may provide storage of computer readable instructions, data structures, program modules, and other data for device 141. For example, hard drive 138 may store operating system 158, application program 157, program module 156, and program data 155. These components may be or may be related to operating system 125, application program 126, program module 127, and program data 128. For example, program module 127 may be created by device 141 when device 141 may load program module 156 into RAM 160.

A user may enter commands and information into the device 141 through input devices such as keyboard 151 and pointing device 152. Pointing device 152 may be a mouse, a trackball, a touch pad, and/or the like. Other input devices (not shown) may include a microphone, joystick, game pad, scanner, and/or the like. Input devices may be connected to user input interface 136 that may be coupled to system bus 121. This may be done, for example, to allow the input devices to communicate with processing unit 159. User input interface 136 may include a number of interfaces or bus structures such as a parallel port, a game port, a serial port, a USB port, and/or the like.

Device 141 may include graphics processing unit (GPU) 129. GPU 129 may be connected to system bus 121. GPU 129 may provide a video processing pipeline for high speed and high-resolution graphics processing. Data may be carried from GPU 129 to video interface 132 via system bus 121. For example, GPU 129 may output data to an audio/video port (A/V) port that may be controlled by video interface 132 for transmission to display device 142.

Display device 142 may be connected to system bus 121 via an interface such as a video interface 132. Display device 142 may be a liquid crystal display (LCD), an organic light-emitting diode (OLED) display, a touchscreen, and/or the like. For example, display device 142 may be a touchscreen that may display information to a user and may receive input from a user for device 141. Device 141 may be connected to peripheral 143. Peripheral interface 133 may allow device 141 to send data to and receive data from peripheral 143. Peripheral 143 may include an accelerometer, an e-compass, a satellite transceiver, a digital camera (for photographs or video), a USB port, a vibration device, a television transceiver, a hands free headset, a Bluetooth® module, a frequency modulated (FM) radio unit, a digital music player, a media player, a video game player module, a speaker, a printer, and/or the like.

Device 141 may operate in a networked environment and may communicate with a remote computer such as device 146. Device 146 may be a computer, a server, a router, a tablet, a smart phone, a peer device, a network node, and/or the like. Device 141 may communicate with device 146 using network 149. For example, device 141 may use network interface 137 to communicate with device 146 via network 149. Network 149 may represent the communication pathways between device 141 and device 146. Network 149 may be a local area network (LAN), a wide area network (WAN), a wireless network, a cellular network, and/or the like. Network 149 may use Internet communications technologies and/or protocols. For example, network 149 may include links using technologies such as Ethernet, IEEE 802.11, IEEE 806.16, WiMAX, 3GPP LTE, 5G New Radio (5G NR), integrated services digital network (ISDN), asynchronous transfer mode (ATM), and/or the like. The networking protocols that may be used on network 149 may include the transmission control protocol/Internet protocol (TCP/IP), the hypertext transport protocol (HTTP), the simple mail transfer protocol (SMTP), the file transfer protocol (FTP), and/or the like. Data exchanged may be exchanged via network 149 using technologies and/or formats such as the hypertext markup language (HTML), the extensible markup language (XML), and/or the like. Network 149 may have links that may be encrypted using encryption technologies such as the secure sockets layer (SSL), Secure HTTP (HTTPS) and/or virtual private networks (VPNs).

Device 141 may include NTP processing device 100. NTP processing device may be connected to system bus 121 and may be connected to network 149. NTP processing device 100 may have more than one connection to network 149. For example, NTP processing device 100 may have a Gigabit Ethernet connection to receive data from the network and a Gigabit Ethernet connection to send data to the network. This may be done, for example, to allow NTP processing device 100 to timestamp data packets at line rate throughput.

As disclosed herein, artificial intelligence may be used to provide a probabilistic interpretation of scores. For example, the artificial intelligence may provide probabilistic reasoning with (e.g., using) complex human-generated and sensed observations. A score used for probabilistic interpretation may be a log base 10 of a probability ratio. For example, scores in a model may be log base 10 of a probability ratio (e.g., similar to the use of logs in decibels or the Richter scale), which provides an order-of-magnitude interpretation to the scores. Whereas the probabilities in a conjunction may be multiplied, the scores may be added.

A score used for probabilistic interpretation may be a measure of surprise; so that a model that makes a prediction (e.g., a surprising prediction) may get a reward for the prediction, but may not get much of a reward for making a prediction that would be expected (e.g., would normally be expected) to be true. For example, a prediction that is usual and/or rate may or may not be unexpected or surprising, and a score may be designed to reflect that. A surprise or unexpected prediction may be relative to a normal. For example, in probability, the normal may be an average, but it may be some other well-defined default, which may alleviate a need for determining the average.

A model with attributes may be used to provide probabilistic interpretation of scores. One or more values or numbers may be specified for an attribute. For example, two numbers may be specified for an attribute (e.g., each attribute) in a model; one number may be applied when the attribute is present in an instance of the model, and the other number may be when the attribute is absent. The rewards may be added to get a score (e.g., total score). In many cases, one of these may be small enough so that it may be effectively ignored, except for cases where it may be the differentiating attribute (in which case it may be a small e value such as 0.001). If the model does not make a prediction about an attribute, that attribute may be ignored.

To provide probabilistic interpretation; of scores, semantics and scores may be used. For example, a semantics for the rewards and scores may provide a principled way to judge correctness and to learn the weights from statistics of the world.

A matcher program may be used to recurse down one or more models (e.g., the hypotheses) and the instances (e.g., the observations) and may sum the rewards/surprises it may encounter. This may be done, for example, such that a model (e.g., the best model) is the one with the highest score, where score may be the sum of rewards. A challenge may be to have a coherent meaning for the rewards that may be added to give scores that makes sense and may be trained on real data. This is non-trivial as there are many complex ideas that may be interacting, and they may math may need to be adjust such that the numbers may make sense to a user.

As disclosed herein, scores may be placed on a secure theoretical framework. The framework may allow the meaning of the scores to be explained. The framework may also allow learning, such as prior and/or expert knowledge, from the data to occur. The framework may allow for unexpected answers to be investigated and/or debugged. The framework may allow for correct reasoning from one or more definitions to be derived. The framework may allow for trick cases to fall out. For example, the framework may help isolate and/or eliminate one or more cases (e.g., special cases). This may be done, for example, to avoid ad hoc adjustments, such as user defined weightings, for the one or more cases.

The framework provided may for compatibility. For example, the framework may allow for the reinterpretation of numbers rather than a rewriting of software code. The framework may allow for the usage of previous scores that may have been based on qualitative probabilities (e.g., kappa-calculus), based on order of magnitude probabilities (but may have drifted). The framework may allow for additive scores, probabilistic interpretation, and/or interactions with ontologies (e.g., including both kind-of and part-of and time).

An attribute of a model may be provided. The attribute may be a property-value pair.

An instance of a model may be provided. An instance may be a description of an item that may have been observed. For example, the instance, may be a description of a place on Earth that has been observed. The instance may be a sequence or tree of one or more attributes, where an attribute (e.g., each attribute) may be labelled present or absent. As used with regard to an attribute, absent may indicate that the attribute may have been evaluated (e.g., explicitly evaluated) and may have been found to be false. For example, “has color green absent” may indicate that it may have been observed that an object does not have the attribute a green color (e.g., the object does not have a green color). With regards to attribute, absent may be different from missing. For example, missing attribute may occur when the attribute may not have been mentioned. As described herein, attributes may be “observed,” where an observation may be part of a vocabulary of probabilities (e.g., a standard vocabulary of probabilities).

A context of an attribute in a model or an instance may be where it occurs. For example, it may be the attributes, or a subset of the attributes, that may come before it in a sequence. For example, the instance may have “there is a room,” “the color is red,” “there is a door,” “the color is green.” In the example, the context may mean that the room is red and the door (in the room) is green.

A model may be a description of what may be expected to be true if an instance matches a model. The model may be a sequence or tree of one or more attributes, where an attribute (e.g., each attribute) may be labeled with a qualitative measure of how confident it may predict some attributes.

A default may be a distribution (e.g., a well-defined distribution) over one or more property values. For example, in geology, it may the background geology. A default may be a value that may not specify anything of interest. A default may be a reference point to which one or more models may be compared. A default distribution may allow for a number of methods and/or analysis as described herein to be performed on one or more models. For example, as described herein, calibration may allow a comparison of one or more models that may be defined with different defaults. A default may be defined but may not need to be specified precisely; for example, a default may be a region that is within 20 km of Squamish.

Throughout this disclosure, the symbol “∧” is used for “and” and “¬” for “not.” In a conditional probability| means “given.” The conditional probability P(m|a∧c) may be “the probability of m given a and c are observed.” If A c may be all that is observed, P(m Iu A c) may be referred to as the posterior probability of m. The probability of m before anything may be observed, may be referred to as the prior probability of m, and may be written P(m), and may be the same as P(m|true).

A model m and attribute a may be specified in an instance in context c. The context may specify where an attribute appears in a model (e.g., in a particular mineralization). In an embodiment, the context c may have been taken into account and the probability of m given c, namely P(m|c), may have been calculated. When a may be observed (e.g., the new context may be a∧c), the probability may be updated using Bayes rule:

$$P ( m \vert a ∧ c ) = \frac{P ( a \vert m ∧ c )}{P ( a \vert c )} P ( m \vert c )$$

It may be difficult to estimate the denominator P(a|c), which may be referred to as the partition function in the machine. The numerator, P(a|m∧c) may also be difficult to assess, particularly if a may not be relevant to m. For example, Jurassic may be observed and c may be empty):

$$P ( {m}_{1} \vert Jurassic ) = \frac{P ( J u r a s sic \vert {m}_{1} )}{P ( Jurassic )} * P ( {m}_{1} )$$

The numerator might be estimated because it may rely on knowing about (e.g., only knowing about) m₁. The denominator, P(Jurassic), may have to be averaged over the Earth, (e.g., all of the Earth) and the probability may depend on the depth in the Earth that we may be considering. And the denominator may be difficult to estimate.

Instead of using (e.g., directly using) the probability of m₁, m₁ may be compared to some default model:

$$\frac{P ( m \vert α ∧ c )}{P ( d \vert α ∧ c )} = \frac{P ( α \vert m ∧ c )}{P ( α \vert d ∧ c )} * \frac{P ( m \vert c )}{P ( d \vert c )} \qquad Equation ( 1 )$$

In Equation (1), P(a|c) may cancel in the division. Instead of estimating the probability, the ratios may be estimated.

The score of a model, and for the reward of an attribute a given a model m in a context c (where the model may specify how the attributes of the instance update the score of the model) may be provide as follows:

$${score}_{d} ( m \vert c ) = {log}_{10} \frac{P ( m \vert c )}{p ( d \vert c )} \\ {reward}_{d} ( α \vert m , c ) = {log}_{10} \frac{P ( α \vert m ∧ c )}{P ( α \vert d ∧ c )}$$

As disclosed herein, a reward may be a function of four arguments: d, a, m and c. It may be described in this manner because it may be the reward of attribute a given model m and context c, with the default d. When c may be empty (or the proposition may be true) the last argument may sometimes be omitted. When d is empty, it may be understood by context and it may also be omitted.

As disclosed herein, the logs used may be in base 10 to aid in interpretability (such as, for example, in decibels and the Richter scale). For simplicity, the base will be omitted for the remainder of this disclosure. It is noted that although base 10 may be used, other bases may be used. rest of this paper.

When there may be a fixed default, the d subscript may be omitted and understood from context. The default d may be included when dealing with multiple defaults, as described herein.

Taking logarithms of Equation (1) gives:

score(m|a∧c)=reward(a|m,c)+score(m|c)

This may indicate how the score may be updated when a is processed. This may imply that the score may be the sum of the rewards from one or more attributes (e.g., each of the attributes) in the instance. If the instance a₁ . . . aₖ is observed, then the rewards may be summed up, where the context cᵢ may be the previous as (e.g., cᵢ=a₁ . . . aᵢ₋₁):

$$score ( m \vert {α}_{1} ∧ … ∧ {α}_{k} ) = {\sum }_{i = 1}^{k} reward ( {α}_{i} \vert m , {c}_{i} ) + s c o r e ( m ) \qquad Equation ( 2 )$$

where score(m) may be the prior for the model (score(m)=log P(m)), and cᵢ may be the context in the model given a₁ . . . aᵢ₋₁ may have been observed.

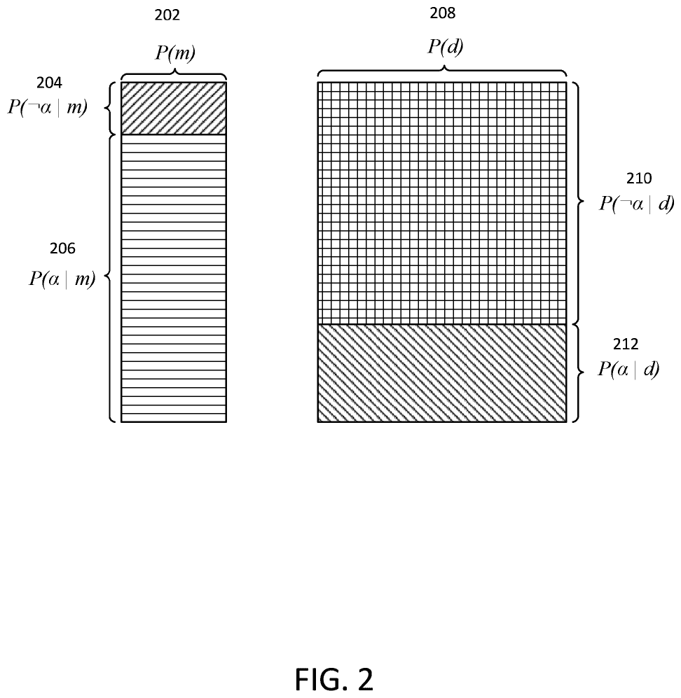

FIG. 2 shows an example of joint probability generated by the probabilistic reasoning embodiments disclosed herein. For example, FIG. 2 may show probabilities figuratively. For purposes of simplicity, FIG. 2 is shown with c omitted.

There may be four regions in FIG. 2, such as 204, 206, 210, and 212. Region 206 may be where a∧m is true. The area of region 206 may be P(a∧m)=P(a|m)*P(m). Region 204 may be where ¬a∧m is true. The area of the region 204 may be P(¬a∧m)=P(¬a|m)*P(m)=(1−P(a|m))*P(m). Region 212 may be where a∧d is true. The area of the region 212 may be P(a∧d)=P(a|d)*P(d). The region 210 may be where ¬a∧d is true. The area of the region 210 may be P(¬a∧d)=P(¬a|d)*P(d)=(1−P(a|d))*P(d). m may be true in region 204 and region 206. a may be true in the region 206 and region 212.

P(m)/P(d) is the ratio of the left area at 202 to the right area at 208. When a is observed, the areas at 204 and 210 may vanish, and the P(m|a)/P(d|a) becomes the ratio of the area at 206 to the area at 212. When ¬a is observed, the areas at 206 and 212 may vanish, and the P(m|¬a)/P(d|¬a) becomes the ratio of the area at 204 to the area at 210. Whether these ratios may be bigger or smaller than P(m)/P(d) may depend on whether the height of area 206 is bigger or smaller than the height of the area 212.

For an attribute a, the probability given the model may be the same as the default, for example P(a∧m∧c)=P(a|d∧c), then the reward may be 0, and it may be assumed that the model may not mention a in this case. Put the other way, if a is not mentioned in the current context, this may mean P(a|m∧c)=P(a|d∧c).

The reward (a|m, c) may tell us how much more likely a may be, in context c, given the model was true, than it was in the background.

Table 1 shows mapping rewards and/or score for probability ratios that may be associated with FIG. 2. In Table 1, the ratio may be as follows:

$$Ratio = \frac{P ( α \vert m ∧ c )}{P ( α \vert d ∧ c )}$$

As shown in Table 1, English labels may be provided. The English labels may assist in interpreting the reward, scores, and/or ratios. The English labels are not intended to be final descriptions. Rather, the English labels are provided for illustrative purposes. Further, the English labels may not be provided for all values. For example, it may not be possible to have reward=3 unless P(a|d∧c)≤0.001. Thus, if a may be likely in the default (e.g., more than 1 in a thousand), then a model may not have a reward of 3 even if may always be true given the model.

Table 1 may allow for the difference in final scores between models to be interpreted. For example, if one model has a score that is 2 more than another, it may mean it is 2 orders of magnitude, or 100 times, more likely. If a model has a score that is 0.6 more than the other it may be about 4 times as likely. If a model has a score that is 0.02 more, then it may be approximately 5% more likely.

TABLE 1
Mapping rewards and/or scores to possibility
ratios and English labels

| Reward | Ratio | | English Label |
|---|---|---|---|
| 3 | 1000.00:1 | 1:0.001 | |
| 2 | 100.00:1 | 1:0.01 | |
| 1 | 10.00:1 | 1:0.10 | strong positive |
| 0.8 | 6.31:1 | 1:0.16 | |
| 0.6 | 3.98:1 | 1:0.25 | |
| 0.4 | 2.51:1 | 1:0.40 | |
| 0.2 | 1.58:1 | 1:0.63 | weak positive |
| 0.1 | 1.26:1 | 1:0.79 | |
| 0.02 | 1.047:1 | 1:0.955 | very weak positive |
| 0 | 1.00:1 | 1:1.00 | not indicative |
| 0.02 | 0.955:1 | 1:0.047 | very weak negative |
| −0.1 | 0.79:1 | 1:1.26 | |
| −0.2 | 0.63:1 | 1:1.58 | weak negative |
| −0.4 | 0.40:1 | 1:2.51 | |
| −0.6 | 0.25:1 | 1:3.98 | |
| −0.8 | 0.16:1 | 1:6.31 | |
| −1 | 0.10:1 | 1:10.00 | strong negative |
| −2 | 0.01:1 | 1:100.00 | |
| −3 | 0.001:1 | 1:1000.00 | |

Qualitative values may be provided. As disclosed herein, English labels may be associated with rewards, ratios, and/or scores. These English labels may be referred to as qualitative values. A number of principles may be associated with qualitative values. Qualitative values that may be used may have the ability to be measured. Instead of measuring these values, the qualitative values may be assigned a meaning (e.g., a reasonable meaning). For example, a qualitative value may be given a meaning such as “weak positive.” This may be done, for example, to provide an approximate value that may be useful and may give a result (e.g., a reasonable result). The qualitative values may be calibrated. For example, the mapping between English labels and the values may be calibrated based on a mix of expert opinion and/or data. This may be approximate as terms (e.g., all terms) with the same word may be mapped to the same value.

The measures may be refined, for example, when there are problems with the results. As an example, a cost-benefit analysis may be performed to determine whether it is worthwhile to find the real values versus approximate values. It may be desirable to avoid a need for one or more accurate measurements (e.g., all measurements to be accurate), which may not be possible due to finite resources. A structure, such as a general structure, may be sufficient and may be used rather than a detailed structure. A more accurate measure may or may not make a difference to the solution.

Statistics and other measurements may be used to provide probabilistic reasoning and may be used when available. The embodiments disclosed herein may provide an advantage over a purely qualitative methodology in that the embodiments may integrate with data (e.g., real data) when it is available.

One or more defaults may be provided. The default d may act like a model. The default d may make a probabilistic prediction for a possible observation (e.g., each possible observation). An embodiment may not make a zero probability for a prediction (e.g., any prediction) that may be possible. Default d may depend on a domain. A default may be selected for a domain, and the default may be changed as experienced is gained in that domain. A default may evolve as experience may be gained.

For example, for modelling landslides in British Colombia (BC) it may be the distribution of feature values in an area, which may be small and well-understood area, such as the area around Squamish, BC, may be diverse. And the area may be used as a default. But the default area may need some small probabilities for observations.

The default may not make any zero probabilities, which may be because diving by zero is not permissible. An embodiment may overcome this by incorporating sensor noise for values that may not be in the background. For example, if the background does not include any gold, then P(gold|d) may be the background level of gold or a probability that gold may be sensed even if there may be a trace amount there.

Default d may be treated as independently predicting a value (e.g., every value). For example, the features may be conditionally independent given the model. The dependence of features may be modeled as described herein.

Negation may be provided. Probabilistic reasoning may be provided when attributes, whether or not the attributes are positive, are observed or missing. If a negation of an attribute is observed, where a reward for the attribute may be given, there may not be enough information to compute the score. When ¬a may be observed, an update rule may be:

$$\frac{P ( m \vert ¬ α ∧ c )}{P ( d \vert ¬ α ∧ c )} = \frac{P ( ¬ α \vert m ∧ c )}{P ( ¬ α \vert d ∧ c )} * \frac{P ( m \vert c )}{P ( d \vert c )} = \frac{1 - P ( α \vert m ∧ c )}{1 - P ( α \vert d ∧ c )} * \frac{P ( m \vert c )}{P ( d \vert c )}$$

### Thus

$$reward ( ¬ α \vert m , c ) = log \frac{1 - P ( α \vert m ∧ c )}{1 - P ( α \vert d ∧ c )}$$

Table 3 may show the positive and negative reward for an example default value. As shown in Table 3, as P(a|m) gets closer to zero, the negative reward may reach a limit. As P(a|m) gets closer to one, the negative reward may approach a negative infinity.

Knowing

$$\frac{P ( α \vert m ∧ c )}{P ( α \vert d ∧ c )}$$

may not provide enough information to compute

$$\frac{1 - P ( α \vert m ∧ c )}{1 - P ( α \vert d ∧ c )} .$$

The relationship may be given by Theorem 1 that follows:

Theorem 1. If 0<P (a|d∧c)<1 (and both fractions may be well-defined):

$$( a ) \frac{P ( α \vert m ∧ c )}{P ( α \vert d ∧ c )} = iff \frac{1 - P ( α \vert m ∧ c )}{1 - P ( α \vert d ∧ c )} = 1 \\ ( b ) \frac{P ( α \vert m ∧ c )}{P ( α \vert d ∧ c )} > 1 iff \frac{1 - P ( α \vert m ∧ c )}{1 - P ( α \vert d ∧ c )} < 1$$

(c) The above two may be the only constraints on these. For any assignment to

$$\frac{P ( α \vert m ∧ c )}{P ( α \vert d ∧ c )} ,$$

and for any number η>0 that obeys the top two conditions, it may be possible that

$$\frac{1 - P ( α \vert m ∧ c )}{1 - P ( α \vert d ∧ c )} = η .$$

### Proof.

$$( a ) \frac{P ( α \vert m ∧ c )}{P ( α \vert d ∧ c )} = 1 iff P ( α \vert m ∧ c ) = P ( α \vert d ∧ c ) iff 1 - P ( α \vert m ∧ c ) = 1 - P ( α \vert d ∧ c ) iff \frac{1 - P ( α \vert m ∧ c )}{1 - P ( α \vert d ∧ c )} = 1 ( b ) \frac{P ( α \vert m ∧ c )}{P ( α \vert d ∧ c )} > 1 iff P ( α \vert m ∧ c ) > P ( α \vert d ∧ c ) iff 1 - P ( α \vert m ∧ c ) < 1 - P ( α \vert d ∧ c ) iff \frac{1 - P ( α \vert m ∧ c )}{1 - P ( α \vert d ∧ c )} < 1 ( c ) Let \frac{P ( α \vert m ∧ c )}{P ( α \vert d ∧ c )} = ζ . So P ( α \vert m ∧ c ) = P ( α \vert d ∧ c ) ζ .$$

Consider the function –(x)=(1−xζ)/(1−x), (where x is P(a|d∧c)) This function is continuous in x, where 0≤x<1. When x→0, f(x)→1. Consider the case where ζ<1, then as x→1 the numerator is bounded away from zero and the denominator approached zero, so the fraction approaches infinity. Because the function is continuous, it takes all values greater than 1. If ζ=1, this is covered by the first case. If ζ>1, x cannot take all values, and f(x) must be truncated at 0, but f is continuous and takes all values between 1 and 0.

In the proof of part (c) above, the restriction on x may be reasonable. If a may be 10 times as likely as b, then b may have a probability of at most 0.1

Theorem 1 may be translated into the following rewards:
(a) reward(a|m, c)=0 if reward(¬a|m, c)=0
(b) reward(a|m, c)>0 if reward(¬a|m, c)<0
(c) reward(a|m, c) and reward(¬a|m, c) may take any values that do not violate the above two constraints.

In some embodiments,

$$\frac{P ( α \vert m ∧ c )}{P ( α \vert d ∧ c )}$$

and P(a|d∧c), or both

$$\frac{P ( α \vert m ∧ c )}{P ( α \vert d ∧ c )} and \frac{1 - P ( α \vert m ∧ c )}{1 - P ( α \vert d ∧ c )}$$

(or their reward equivalent) may be specified. In other embodiments, it may not be specified and some assumptions (e.g., reasonable assumptions) may be made. For example, these may rely on a rule that if x is small then (1−x)≈1, and that dividing or multiplying by something close to 1, may not make much difference, except for cases where everything else may be equal, in which case whether the ratio may be bigger than 1 or less than 1 may make the difference as to which may be better.

The probabilistic reasoning embodiments described herein may be applicable to a number of scenarios and/or industries, such as medicine, healthcare, insurance markets, finance, land use planning, environmental planning, real estate, mining, and/or the like. For example, probabilistic reasoning may be applied to mining such that a model for a gold deposit may be provided.

A model of a gold deposit may include one or more of the following:
Has Genetic Setting—Greenstone—Always; Present: strong positive; Absent: strong negative
Element Enhanced to Ore—Au—Always; Present: strong positive; Absent: strong negative
Mineral Enhanced to Ore—Electrum—Sometimes; Present: strong positive; Absent: weak negative
Element Enhanced—As—Usually; Present: weak positive; Absent: weak negative

An example instance for the gold deposit model may be as follows:
Has Genetic Setting—Greenstone—Present
Element Enhanced to Ore—Au—Present
Mineral Enhanced to Ore—Electrum—Absent
Element Enhanced—As—Present

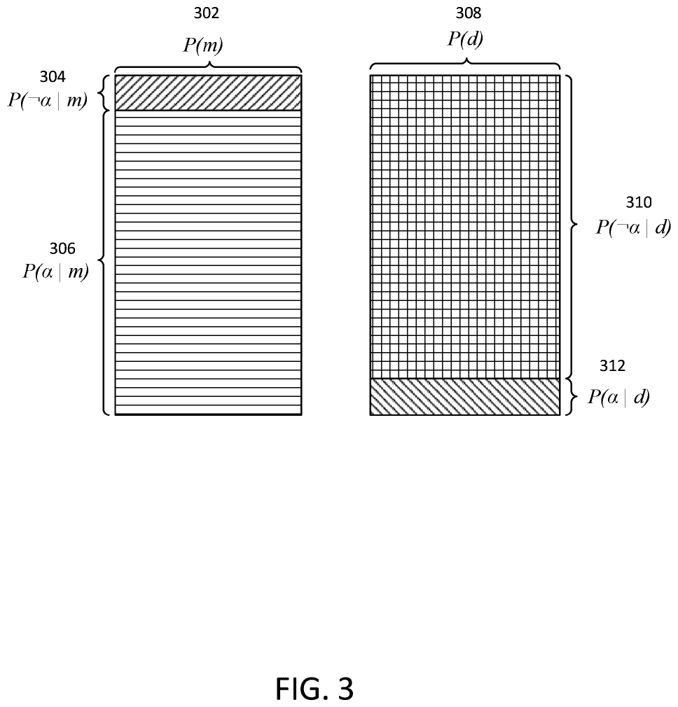

FIGS. 3-13 may reflect the rewards in the heights. But these figures may not reflect the scores in the widths. Given the rewards, the frequencies may be computed. FIGS. 3-13 may use the computed frequencies and may not use the stated frequencies. In FIGS. 3-13, the depicted heights may be accurate, but the widths may not have a significance.

The following description describes how the embodiments disclosed herein may be applied to provide probabilistic reasoning for the mining industry for illustrative purposes. The embodiments described herein may be applied to other industries such as medicine, finance, law, threat detection for computer security, and/or the like.

FIG. 3 shows an example depiction of a probability of an attribute in part of a model for a gold deposit. The model may have a genetic setting. The part of the model may be depicted as the following, where attribute a may be “Has Genetic Setting,” which may be “Greenstone”. FIG. 3 may depict the attribute a as “present: strong positive; absent: strong negative.” For example, the presence of greenstone may indicate a strong positive in the model for a gold deposit. The absence of greenstone may indicate a strong negative in the model for the gold deposit.

As shown in FIG. 3, the attribute a has been observed. At 302, the probability of an attribute in the model may be shown. At 304, the absence of the attribute greenstone may indicate a strong negative in the model for the gold deposit. At 306, the presence of the attribute greenstone may indicate a strong positive in the model for the gold deposit. At 308, the probability of an attribute in a default may be shown. An absence of the attribute greenstone in the default may provide a probability at 310. A presence of the attribute greenstone in the default may provide a probability at 312. In FIG. 3, the reward may be reward(Genetic_setting=greenstone|m)=1.

A second observation may be “Element Enhanced to Ore—Au—Present”. For example, the model may be used to determine a probability of a gold deposit given the presence and/or absence of AU. In the example model Au may frequently be found (e.g., always found) with gold. The presence of the attribute Au may indicate a strong positive. The absence of the attribute Au may indicate a strong negative. And the model may be depicted in a similar way as the genetic setting, with a being Au_enhanced_to_ore.

For example, in FIG. 3, a may be Au_enhanced_to_ore. As shown in FIG. 3, the attribute a has been observed. At 302, the probability of an attribute in the model may be shown. At 304, the absence of the attribute Au may indicate a strong negative in the model for the gold deposit. At 306, the presence of the attribute Au may indicate a strong positive in the model for the gold deposit. At 308, the probability of an attribute in a default may be shown. An absence of the attribute Au in the default may provide a probability at 310. A presence of the attribute Au in the default may provide a probability at 312. In FIG. 3, the reward may be reward(Au_enhanced_to_ore|m)=1.

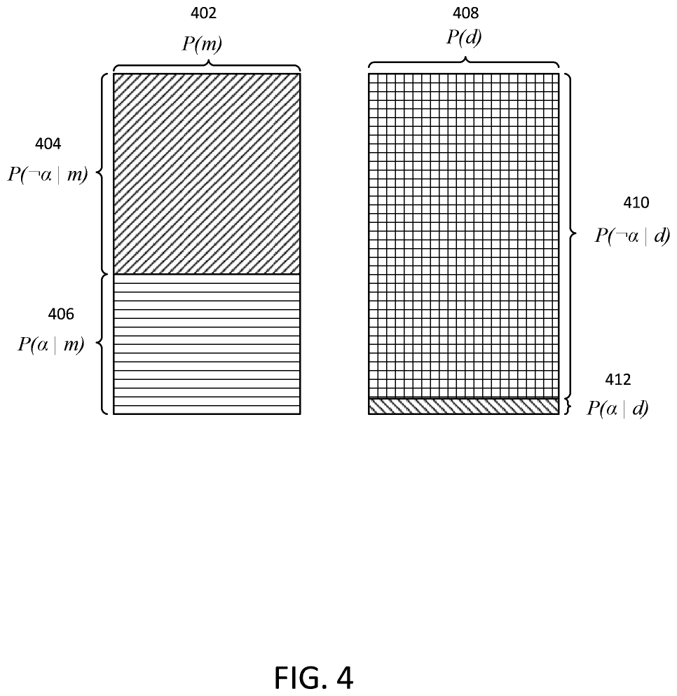

FIG. 4 shows an example depiction of a probability of an attribute in part of a model. The model may be for a gold deposit. The model may indicate a presence of an attribute may be a strong positive. The model may indicate that an absence of an attribute may be a weak negative. In a model for gold deposit, the attribute may be Electrum. For example, Electrum enhanced to Ore that is absent may be considered.

A model may be shown in FIG. 4, where the presence of an attribute may indicate a strong positive and an absence of the attribute may indicate a weak negative. At 402, a probability for the attribute in the model may be provided. At 404, the absence of the attribute in the model may indicate a weak negative. At 406, the presence of the attribute in the model may indicate a strong positive. At 408, a probability for the attribute in a default may be provided. At 410, a probability for the absence of the attribute in the default may be provided. At 412, a probability for the presence of the attribute in the default may be provided.

Using the gold deposit model discussed herein, the attribute may be Electrum. For example, where a may be Electrum_enhanced_to_ore, and ¬a may have been observed. Electrum may provide weak negative evidence for the model, for example, evidence that the model may be less likely. The reward may be reward(Electrum_enhanced_to_ore=absent|m)=−0.2.

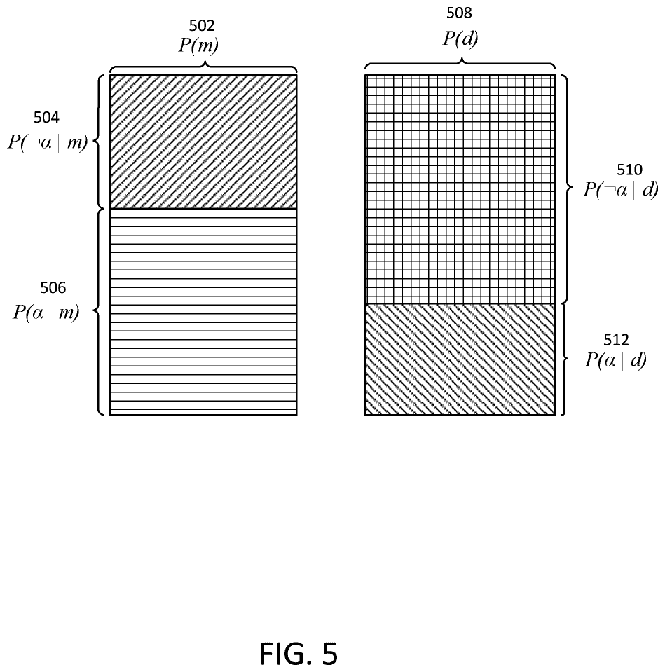

FIG. 5 shows another example depiction of a probability of an attribute in part of a model. The model may be for a gold deposit. The model may indicate a presence of an attribute may be a weak positive. The model may indicate that an absence of an attribute may be a weak negative. In a model for gold deposit, the attribute may be Arsenic (As).

A model may be shown in FIG. 5, where the presence of an attribute may indicate a weak positive and an absence of the attribute may indicate a weak negative. At 502, the probability of the attribute in the model may be shown. At 504, the absence of the attribute in the model may indicate a weak negative. At 506, the presence of the attribute in the model may indicate a weak positive. At 508, a probability for the attribute in a default may be provided. At 510, a probability for the absence of the attribute in the default may be provided. At 512, a probability for the presence of the attribute in the default may be provided.

Using the gold deposit model discussed herein, the attribute may be As. For example, where a may be As_enhanced and a may have been observed. As may provide weak positive evidence for the model, for example, evidence that the model may be more likely. A model with as present may indicate a weak positive and a model with as absent may indicate a weak negative. In FIG. 5, the reward may be reward(As_enhanced=present|m)=0.2.

Summing the rewards from FIGS. 3-5 may give a total reward. For example, the following may be added together to produce a total reward:

reward(Genetic_setting=greenstone|m)=1

reward(Au_enhanced_to_ore|m)=1

reward(Electrum_enhanced_to_ore=absent|m)=−0.2

reward(As_enhanced=present|m)=0.2

Considering the above, a total reward may be 1+1−0.2+0.2=2.0. The total reward may indicate that the evidence in the instance makes this model 100 times more likely than before the evidence.

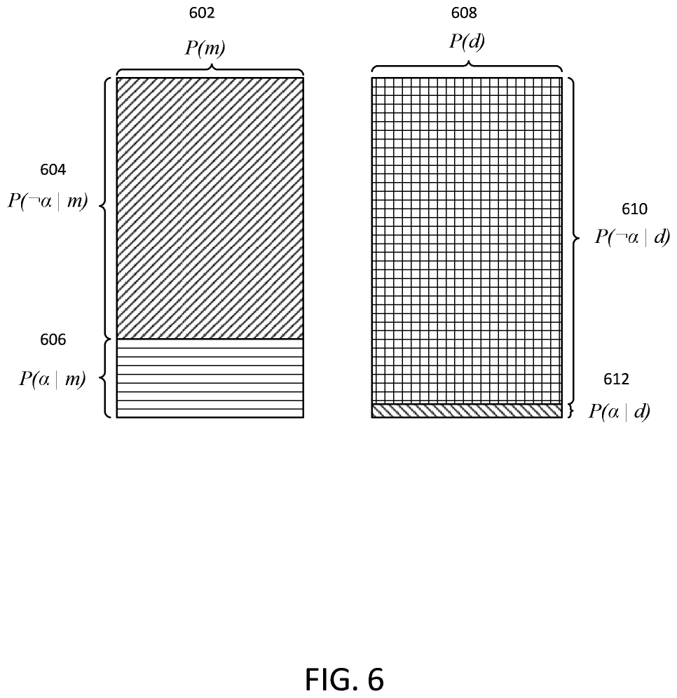

FIG. 6 shows an example depiction of a probability of an attribute that may be rare for a model and may be rare in the background. The model may indicate a presence of an attribute may be a weak positive. The model may indicate that an absence of an attribute may be a weak negative.

A model may be shown in FIG. 6, where the presence of an attribute may indicate a weak positive and an absence of the attribute may indicate a weak negative. At 602, the probability of the attribute in the model may be shown. At 604, the absence of the attribute in the model may indicate a weak negative. At 606, the presence of the attribute in the model may indicate a weak positive. At 608, a probability for the attribute in a default may be provided. At 610, a probability for the absence of the attribute in the default may be provided. At 612, a probability for the presence of the attribute in the default may be provided.

As shown in FIG. 6, a may be rare both for the case where m is true and in the background. As may mean that, in this case, both the numerator and denominator may be close to 1. So, the ratio may be close to 1 and the reward may be close to 0.

If the probability of an attribute in the model is greater than the probability in the default, the reward for present may be positive and the reward for absent may be negative. If the probability of an attribute in the model is less than the probability in the default, the reward for present may be negative and the reward for absent may be positive. If the probabilities may be the same, the model may not need to mention the attribute.

For example, if some mineral is rare whether or not the model is true (even though the mineral may be, say, 10 times as likely if the model is true, and so it provides evidence for the model), the absence of the mineral may be common even if the model may be true. So, observing the absence of the mineral may provide some, but weak (e.g., very weak), evidence that the model is false.

As another example follows:

$$Suppose reward ( α \vert m , c ) = 2 , so \frac{P ( α \vert m ∧ c )}{P ( α \vert d ∧ c )} = 100.$$

Suppose P(a|d∧c)=10⁻⁴.
Then P(a|m∧c)=100*10⁻⁴=10⁻²
Then

$$\begin{matrix}reward ( ¬ α \vert m , c ) = log \frac{1 - P ( α \vert m ∧ c )}{1 - P ( α \vert d ∧ c )} \\ = log \frac{1 - {10}^{- 2}}{1 - {10}^{- 4}} \\ = log 0.990099 \\ = - 0.004321\end{matrix}$$

The reward for ¬a is aways close to zero if a is rare, whether or not the model holds. (It may be easier to ignore the reward and give it some small±ϵ).

In the example above, the ratio

$$\frac{1 - P ( α \vert m ∧ c )}{1 - P ( α \vert d ∧ c )}$$

is close to 1, and so the score of ¬a is close to zero, but is of the opposite sign of the score of a. It may not be worthwhile to record these. Instead, a value, such as ±0.01, may be used. And the value may make a difference when one model may have this as an extra condition (e.g., the only extra condition).

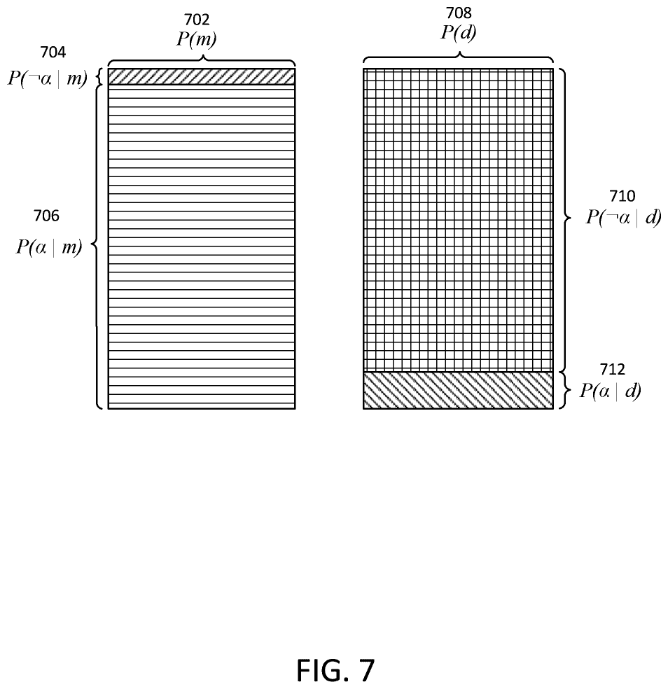

FIG. 7 shows an example depiction of a probability of an attribute that may be rare in the background and may not be rare in a model. The model may indicate a presence of an attribute may be a strong positive. The model may indicate that an absence of an attribute may be a strong negative.

A model may be shown in FIG. 7, where the presence of an attribute may indicate a strong positive and an absence of the attribute may indicate a strong negative. At 702, the probability of the attribute in the model may be shown. At 704, the absence of the attribute in the model may indicate a strong negative. At 706, the presence of the attribute in the model may indicate a strong positive. At 708, a probability for the attribute in a default may be provided. At 710, a probability for the absence of the attribute in the default may be provided. At 712, a probability for the presence of the attribute in the default may be provided.

As shown in FIG. 7, a may be common where m is true and a may be rare in the background (e.g., the default). The prediction for present observations and absent observations may be sensitive to the actual values.

In an example:

$$Suppose reward ( α \vert m , c ) = 2 , so \frac{P ( α \vert m ∧ c )}{P ( α \vert d ∧ c )} = 100.$$

Suppose P(a|d∧c)=0.00999.
Note that 0.01 is the most it can be.
Then P(a|m∧c)=100*0.00999=0.999
Then

$$\begin{matrix}reward ( ¬ α \vert m , c ) = log \frac{1 - P ( α \vert m ∧ c )}{1 - P ( α \vert d ∧ c )} \\ = log \frac{1 - 0.999}{1 - 0.0999} \\ = log 0.00101009 \\ = - 2.9956\end{matrix}$$

The reward for ¬a may be sensitive (e.g., very sensitive) to P(a|m∧c), and it may better specify both a reward for a and a reward for ¬a.

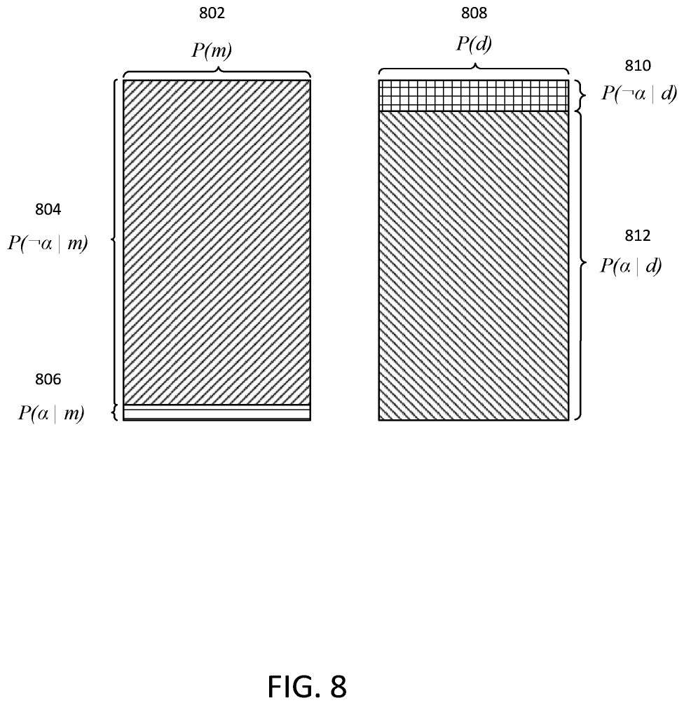

FIG. 8 shows an example depiction of a probability of an attribute that may be common in the background. The model may indicate a presence of an attribute may be a weak positive. The model may indicate that an absence of an attribute may be a weak negative.

A model may be shown in FIG. 8, where the presence of an attribute may indicate a weak positive and an absence of the attribute may indicate a weak negative. At 802, the probability of the attribute in the model may be shown. At 804, the absence of the attribute in the model may indicate a weak negative. At 806, the presence of the attribute in the model may indicate a weak positive. At 808, a probability for the attribute in a default may be provided. At 810, a probability for the absence of the attribute in the default may be provided. At 812, a probability for the presence of the attribute in the default may be provided.

If a is common in the background, there may never be a big positive award for observing a, but there may be a big negative reward.

In an example, the following may be considered:

Suppose P(a|d∧c)=0.9.

The most reward(a|m, c) may be is log 1/0.9≈0.046. So, there may not be (e.g., may never be) a big positive reward for observing a.

In another example, where a may be rare in the model, but may be common in the background, the following may be considered:

Suppose P(a|d∧c)=0.9.

$$Suppose reward ( α \vert m , c ) = - 2. so \frac{P ( α \vert m ∧ c )}{P ( α \vert d ∧ c )} = 0.01 .$$

So P(a|m∧c)=0.009
Then

$$\begin{matrix}reward ( ¬ α \vert m , c ) = log \frac{1 - P ( α \vert m ∧ c )}{1 - P ( α \vert d ∧ c )} \\ = log \frac{1 - 0.009}{1 - 0.9} \\ = log 9.91 \\ = 0.996\end{matrix}$$

This value may be sensitive (e.g., very sensitive) to P(a|d∧c), but may not be sensitive (e.g., may not be very sensitive) to P(a|m∧c). This may be because if P(a|m∧c)≈0, then 1−P(a|m∧c)≈1. It may be better to specify P(a|d∧c), and use that for one or more models (e.g., all models) when ¬a may be observed.

Mapping to and from probabilities and rewards may be provided. For example, of the following four values, any two may be specified and the other two may be derived:
P(a|m∧c)
P(a|d∧c)
reward(a|m, c)
reward(¬a|m, c)

To map to and from the probabilities and rewards, the probabilities may be >0 and 1. It may not be possible to compute the probabilities if the rewards are zero, in which case it may be determined that the probabilities may be equal, but it may not be determined what they are equal to.

The rewards may be derived from the probabilities using the following:

$$reward ( α \vert m , c ) = log \frac{P ( α \vert m ∧ c )}{P ( α \vert d ∧ c )} \\ reward ( ¬ α \vert m , c ) = log \frac{1 - P ( α \vert m ∧ c )}{1 - P ( α \vert d ∧ c )}$$

The probabilities may be derived from the rewards using the following:

$$P ( α \vert d ∧ c ) = \frac{1 - {10}^{reward ( ¬ α \vert m , c )}}{{10}^{reward ( α \vert m , c )} - {10}^{reward ( ¬ α \vert m , c )}} \\ P ( α \vert m ∧ c ) = {10}^{reward ( α \vert m , c )} * \frac{1 - {10}^{reward ( ¬ α \vert m , c )}}{{10}^{reward ( α \vert m , c )} - {10}^{reward ( ¬ α \vert m , c )}}$$

This may indicate that reward(a|m, c)≠reward(¬a|m, c). For example, they may be equal if they are both zero. In this case, there may not be enough information to infer P(a|m∧c), which should be similar to P(a|d, c).

If P(a|m) and reward(a|m, c) may be known, the other two may be computed as:

$$P ( α \vert d ∧ c ) = \frac{P ( α \vert m ∧ c )}{{10}^{reward ( α \vert m , c )}} \\ reward ( ¬ α \vert m , c ) = log \frac{1 - P ( α \vert m ∧ c )}{1 - \frac{P ( α \vert m ∧ c )}{{10}^{reward ( α \vert m , c )}}}$$

While we may derive any two from the other two, often these may not be sensitive (e.g., very sensitive) to one of the values. This may occur, for example, when dividing by a value close to zero, the difference from zero may matter, but if dividing by a value close to one, the distance from one may not matter. In these cases, a large variation may give approximately the same answer. So, it may be better to allow a user to specify the third value, an indicator that there may be an issue with the third value if it results in a large error.

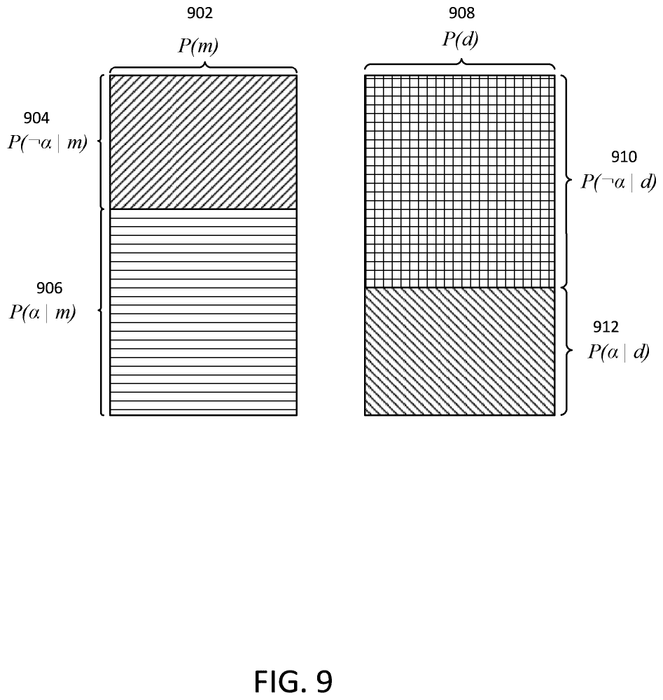

FIG. 9 shows an example depiction of a probability of an attribute, where the presence of the attribute may indicate a weak positive and an absence of the attribute may indicate a weak negative. At 906, a probability of a given model m may be a weak positive. For example, a model with a present may be a weak positive and may have a value of +0.2. At 904, a probability of ¬a given model m may be a weak negative. For example, a model with a absent may be a weak negative and may have a value of −0.2.

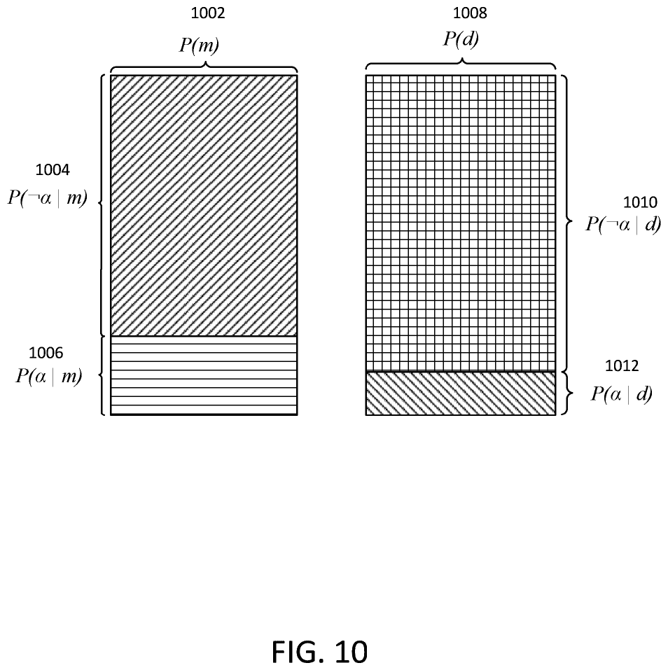

FIG. 10 shows an example depiction of a probability of an attribute, where the presence of the attribute may indicate a weak positive and an absence of the attribute may indicate a weak negative. At 1006, a probability of a given model m may be a weak positive. For example, a model with a present may be a weak positive and may have a value of +0.2. At 1004, a probability of ¬a given model m may be a weak negative. For example, a model with a absent may be a weak negative and may have a value of −0.05.

A user may not be able to ascertain whether the weak negative would be −0.2 or −0.05, but may have some idea that one of these diagrams is more plausible than the other. For example, a user may view FIG. 9 and FIG. 10 and have some idea that FIG. 9 or FIG. 10 may be more plausible than the other for a model.

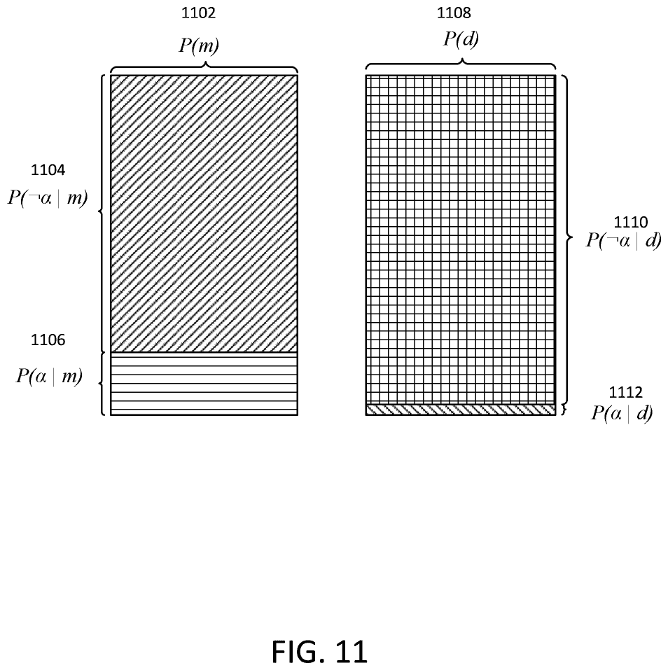

FIG. 11 shows an example depiction of a probability of an attribute, where the presence of the attribute may indicate a strong positive and an absence of the attribute may indicate a weak negative. At 1106, a probability of a given model m may be a strong positive. For example, a model with a present may be a strong positive and may have a value of +1. At 1104, a probability of ¬a given model m may be a weak negative. For example, a model with a absent may be a weak negative and may have a value of −0.01.

From a user standpoint, FIG. 11 may appear to be very different from FIG. 10. But, FIG. 11 and FIG. 10 may have a number of differences, such as between the values of 1004 and 1104, the values of 1006 and 1106, and the values of 1008 and 1108.

As disclosed herein, two out of the four probability/reward values may be used to change from one domain to the other. The decision of which two probability/reward values to use may change from one value to another. The decision of which to use may not affect the matcher. The decision of which to use may affect the user interface, such as how knowledge may be captured, and how solutions may be explained. It may be possible that a tool to capture knowledge, such as expert knowledge (e.g., a doctor, a geologist, a security expert, a lawyer, etc.) may include two or more of the four probability/rewards values (e.g., four).

In an embodiment, the following two probability/rewards values may be preferred:
P(a|d∧c)
reward(a|m, c)

This may be done, for example, such that for an attribute a (e.g., each attribute a), there may be a value per model (e.g., one value per model), which may be about diagnostically, and a global value (e.g., one global value), which may be about probability. The global value may be referred to as a supermodel.

The negative reward, which may be the value used by the matcher when ¬a is observed, may be obtained using:

$$reward ( ¬ α \vert m , c ) = log \frac{1 - P ( α \vert d ∧ c ) {10}^{reward ( α \vert m , c )}}{1 - P ( α \vert d ∧ c )} \qquad Equation ( 3 )$$

In cases where a may be unusual both in the default and the model, the value of P(a|d∧c) may not matter very much. The reward for the negation may be close to zero. For example, as disclosed herein, this may occur where P(a|d∧c) and P(a|m∧c) may both be small.

As disclosed herein, the value of P(a|d∧c) may matter when P(a|d∧c) may be close to one. The value of P(a|d∧c) may when the reward may be big enough such that P(a|d∧c) may be close to 1, in which case it may be better to treat this as a case (e.g., a special case) in knowledge acquisition.

In some embodiments, this may be unreasonable. For example, it may be unreasonable when the negative reward may be sensitive (e.g., very sensitive) to the actual values. This may occur when the P(a|d∧c) may close to 1, as it may cause a division by something close to 0. In that case, it may be better to reason in terms of ¬a rather than a, as further disclosed herein.

In an embodiment, the following may be considered:

$$er ( α \vert m , c ) = {10}^{reward ( α \vert m , c )} = \frac{P ( α \vert m ∧ c )}{P ( α \vert d ∧ c )}$$

This may imply:

P(a|m∧c)=er(a|m,c)*P(a|d∧c)

This may allow for P(a|m∧c) to be replaced by the right-hand side of the equation whenever it may not be provided. For example:

$$\begin{matrix}er ( ¬ α \vert m , c ) = {10}^{reward ( ¬ α \vert m , c )} \\ = \frac{1 - P ( α \vert m ∧ c )}{1 - P ( α \vert d ∧ c )} \\ = \frac{1 - er ( α \vert m , c ) * P ( α \vert d ∧ c )}{1 - P ( α \vert d ∧ c )}\end{matrix}$$

This may (taking logs) give Equation (3). To then derive the other results:

er(¬a|m,c)−er(¬a|m,c)*P(a|d∧c)=1−er(a|m,c)*P(a|d∧c)

Collecting the terms for P(a|d∧c) together may give:

er(a|m,c)*P(a|d∧c)−er(¬a|m,c)*P(a|d∧c)=1−er(¬a|m,c)

Which may provide the following:

$$P ( α \vert d ∧ c ) = \frac{1 - er ( ¬ α \vert m , c )}{er ( α \vert m , c ) - er ( ¬ α \vert m , c )}$$

Which may be one of the formulae. The following may then be derived:

$$P ( α \vert m ∧ c ) = er ( α \vert m , c ) * \frac{1 - er ( ¬ α \vert m , c )}{er ( α \vert m , c ) - er ( ¬ α \vert m , c )}$$

Alternative default ds may be provided. The embodiments disclosed herein may not depend on what d may actually be, as long as P(a|d∧c)≠0 when P(a|m∧c)≠0, because otherwise it may result in divide-by-zero errors. There may be a number of different defaults that may be used.

For example, d may some default model, which may be referred to as the background. This may be any distribution (e.g., well-defined distribution). The embodiments may convert between different defaults using the following:

$$\frac{P ( α \vert m ∧ c )}{P ( α \vert {d}_{1} ∧ c )} = \frac{P ( α \vert m ∧ c )}{P ( α \vert {d}_{2} ∧ c )} * \frac{P ( α \vert {d}_{2} ∧ c )}{P ( α \vert {d}_{1} ∧ c )}$$

To convert between d₁ and d₂,

$$\frac{P ( α \vert {d}_{2} ∧ c )}{P ( α \vert {d}_{1} ∧ c )}$$

may be used for each a where they may be different.

Taking logs may produce the following:

$$log \frac{P ( α \vert m ∧ c )}{P ( α \vert {d}_{1} ∧ c )} = log \frac{P ( α \vert m ∧ c )}{P ( α \vert {d}_{2} ∧ c )} + log \frac{P ( α \vert {d}_{2} ∧ c )}{P ( α \vert {d}_{1} ∧ c )}$$

$${reward}_{d 1} ( α \vert m , c ) = {reward}_{d 2} ( α \vert m , c ) + {reward}_{d 1} ( α \vert {d}_{2} , c )$$

d may be the proposition true. In this case P(d|anything)=1, and Equation (1) may be a standard Bayes rule. In this case, scores and rewards (e.g., all scores and rewards) may be negative or zero, because the ratios may be probabilities and may be less than or equal to 1. The probability of a value (e.g., each value) that may be observed may need to be known.

d may be ¬m. For example, each model m may be compared to ¬m. Then the score may become the log-odds and the reward may become the log-likelihood. There may be a mapping between odds and probability. This may be difficult to assess because ¬m may include a lot of possibilities, which an expert may be reluctant to assess. Using the log-odds may make the model equivalent to a logistic regression model as further described herein.

Conjunctions and other logical formulae may be provided. Sometimes features may operate in non-independent ways. For example, in a landslide, both a propensity and a trigger may be used as one without the other may not result in a landslide. In minerals exploration two elements may provide evidence for a model but observing both elements may not provide twice as much evidence.

As disclosed herein, the embodiments may be able to handle one or more scenarios. They may be expressive (e.g., equally expressive,) and they may work if the numbers may be specified accurately. The embodiment may they differ in what may be specified and may have different qualitative effects when approximate values may be given.

The embodiments disclosed herein may allow for logical formulae in weights and/or conditionals.

For purposes of simplicity, the embodiments may be discussed in terms of two Boolean properties a₁ and a₂. But the embodiments are not limited to two Boolean properties. Rather, the embodiments may operate on one or more properties, which may or may not be Boolean properties. In an example where two Boolean properties are used, each property may be modeled by itself (e.g., when the other may not be observed) and their interaction. To specify arbitrary probabilities on 2 Boolean variables, 3 numbers may be used as there may be 4 assignments of values to the variables. And the probability of the 4th assignment of values may be computed from the other three, as they may sum to 1.

In an example embodiment, the following may be provided:
reward(a₁|m)
reward(a₂|m)
reward(a₁∧a₂|m)

If a₁ may be observed by itself, the model may get the first reward, and if a₁∧a₂ may be observed, it may get all three rewards. The negated cases may be computed from this.

For example, for the following weights and probabilities:

| | |
|---|---|
| reward(α₁ \| m) = w₁ | P(α₁ \| d) = p₁ |
| reward(α₂ \| m) = w₂ | P(α₂ \| d) = p₂ |
| reward(α₁ \hat{ } α₂ \| m) = w₃ | |

a₁ and a₂ may independent given d, but may be dependent given m. w₃ may be chosen. The positive rewards may be additive:
score(m|a₁)=w₁
score(m|a₂)=w₂
score(m|a₁∧a₂)=w₁+w₂+w₃

Then

P(a₁|m)=P(a₁|d)*10^{w}^{₁}=p₁*10^{w}^{₁}

the following weights may be derived:

| | |
|---|---|
| w̅₁ = reward(¬α₁ \| m) | w̅₂ = reward(¬α₂ \| m) |

w̅₁̅ may be derived as follows (because a₂ may be ignored when not observed):

$$\begin{matrix}1 = P ( {α}_{1} \vert m ) + ( ¬ {α}_{1} \vert m ) \\ = P ( {α}_{1} \vert d ) * {10}^{{w}_{1}} + P ( ¬ {α}_{1} \vert d ) * {10}^{\overline{{\overline{w}}_{1}}} \\ = {p}_{1} * {10}^{{w}_{1}} + ( 1 - {p}_{1} ) * {10}^{\overline{{w}_{1}}} \\ \overline{{w}_{1}} = log \frac{1 - {p}_{1} * {10}^{{w}_{1}}}{1 - {p}_{1}}\end{matrix}$$

which may be similar to or the same as Equation (3). Similarly

$$\overline{{w}_{2}} = log \frac{1 - {p}_{2} * {10}^{{w}_{2}}}{1 - {p}_{2}}$$

The scores of other combinations of negative observations may be derived. Let score(m|a₁∧¬a₂)=w₄. w₄ may be derived as follows:

$$\begin{matrix}P ( {α}_{1} \vert m ) = P ( {α}_{1} ∧ {α}_{2} \vert m ) + P ( {α}_{1} ∧ ¬ {α}_{2} \vert m ) \\ {p}_{1} * {10}^{{w}_{1}} = {p}_{1} * {p}_{2} * {10}^{{w}_{1} + {w}_{2} + {w}_{3}} + {p}_{1} * ( 1 - {p}_{2} ) * {10}^{{w}_{4}} \\ {10}^{{w}_{1}} = {p}_{2} * {10}^{{w}_{1} + {w}_{2} + {w}_{3}} + ( 1 - {p}_{2} ) * {10}^{{w}_{4}} \\ {10}^{{w}_{4}} = \frac{{10}^{{w}_{1}} * ( 1 - {p}_{2} * {10}^{{w}_{2} + {w}_{3}} )}{1 - {p}_{2}} \\ {w}_{4} = {w}_{1} + log \frac{( 1 - {p}_{2} * {10}^{{w}_{2} + {w}_{3}} )}{1 - {p}_{2}}\end{matrix}$$

The reward for ¬a₂ in the context of a₁ may be:

$$reward ( ¬ {α}_{2} \vert m , {α}_{1} ) = log \frac{( 1 - {p}_{2} * {10}^{{w}_{2} + {w}_{3}} )}{1 - {p}_{2}}$$

And may not be equal to the reward for ¬a₂ not in that context of a₁. The discount (e.g., the number p₂ may be multiplied by in the numerator) may be discounted by w₃ as well as w₂.

By symmetry:

$$reward ( ¬ {α}_{1} \vert m , {α}_{2} ) = log \frac{( 1 - {p}_{1} * {10}^{{w}_{1} + {w}_{3}} )}{1 - {p}_{1}}$$

The last case may be when both are observed to be negative. For example, let score(m|¬a₁∧¬a₂)=w₅. w₅ may be derived as follows:

$$\begin{matrix}P ( ¬ {α}_{1} \vert m ) = P ( ¬ {α}_{1} ∧ {α}_{2} \vert m ) + P ( ¬ {α}_{1} ∧ ¬ {α}_{2} \vert m ) \\ ( 1 - {p}_{1} ) {10}^{\overline{{w}_{1}}} = ( 1 - {p}_{1} ) * {p}_{2} * {10}^{{w}_{2}} * \frac{( 1 - {p}_{1} * {10}^{{w}_{1} + {w}_{3}} )}{1 - {p}_{1}} + ( 1 - {p}_{1} ) * ( 1 - {p}_{2} ) * {10}^{w 5} \\ {10}^{w 5} = \frac{{10}^{\overline{{w}_{1}}} - {p}_{2} * {10}^{{w}_{2}} * \frac{( 1 - {p}_{1} * {10}^{{w}_{1} + {w}_{3}} )}{1 - {p}_{1}}}{1 - {p}_{2}} \\ = \frac{\frac{1 - {p}_{1} * {10}^{{w}_{1}}}{1 - {p}_{1}} - {p}_{2} * {10}^{{w}_{2}} * \frac{( 1 - {p}_{1} * {10}^{{w}_{1} + {w}_{3}} )}{1 - {p}_{1}}}{1 - {p}_{2}} \\ = \frac{1 - {p}_{1} * {10}^{{w}_{1}} - {p}_{2} * {10}^{{w}_{2}} * ( 1 - {p}_{1} * {10}^{{w}_{1} + {w}_{3}} )}{( 1 - {p}_{1} ) * ( 1 - {p}_{2} )} \\ = \frac{1 - {p}_{1} * {10}^{{w}_{1}} - {p}_{2} * {10}^{{w}_{2}} + {p}_{1} * {p}_{2} * {10}^{{w}_{1} + {w}_{2} + {w}_{3}}}{( 1 - {p}_{1} ) * ( 1 - {p}_{2} )}\end{matrix}$$

Which may be like the product of w̅₁̅ and w̅₂̅ except for the w₃. Note that if w₃ may not be zero it factorizes into two products corresponding to w̅₁̅ and w̅₂̅. It may be computed how much w₃ changes the independence assumption; continuing the previous derivation:

$$\begin{matrix}{10}^{{w}_{5}} = \frac{1 - {p}_{1} * {10}^{{w}_{1}} - {p}_{2} * {10}^{{w}_{2}} + {p}_{1} * {p}_{2} {10}^{{w}_{1} + {w}_{2} + {w}_{3}}}{( 1 - {p}_{1} ) * ( 1 - {p}_{2} )} \\ = \frac{1 - {p}_{1} * {10}^{{w}_{1}} - {p}_{2} * {10}^{{w}_{2}} + {p}_{1} * {p}_{2} {10}^{{w}_{1} + {w}_{2}} - {p}_{1} * {p}_{2} * {10}^{{w}_{1} + {w}_{2}} + {p}_{1} * {p}_{2} * {10}^{{w}_{1} + {w}_{2} + {w}_{3}}}{( 1 - {p}_{1} ) * ( 1 - {p}_{2} )} \\ = \frac{( 1 - {p}_{1} * {10}^{{w}_{1}} ) * ( 1 - {p}_{2} * {10}^{{w}_{2}} ) - {p}_{1} * {p}_{2} * {10}^{{w}_{1} + {w}_{2}} + {p}_{1} * {p}_{2} * {10}^{{w}_{1} + {w}_{2} + {w}_{3}}}{( 1 - {p}_{1} ) * ( 1 - {p}_{2} )} \\ = \frac{( 1 - {p}_{1} * {10}^{{w}_{1}} ) * ( 1 - {p}_{2} * {10}^{{w}_{2}} ) - {p}_{1} * {p}_{2} * {10}^{{w}_{1} + {w}_{2}} ( 1 - {10}^{{w}_{3}} )}{( 1 - {p}_{1} ) * ( 1 - {p}_{2} )} \\ = {10}^{\overline{{w}_{1}}} {10}^{\overline{{w}_{2}}} - \frac{{p}_{1} * {p}_{2} * {10}^{{w}_{1} + {w}_{2}} ( 1 - {10}^{{w}_{3}} )}{( 1 - {p}_{1} ) * ( 1 - {p}_{2} )}\end{matrix}$$

For example, the score may be as follows:

$$score ( m \vert ¬ {α}_{1} ∧ ¬ {α}_{2} ) = log \frac{1 - {p}_{1} * {10}^{{w}_{1}} - {p}_{2} * {10}^{{w}_{2}} + {p}_{1} * {p}_{2} * {10}^{{w}_{1} + {w}_{2} + {w}_{3}}}{( 1 - {p}_{1} ) * ( 1 - {p}_{2} )}$$

There may be some unintuitive consequences of the definition, which may be explained in the following example: Suppose reward(a₁|m)=0, reward(a₂|m)=0 and reward(a₁∧a₂|m)=0.2, and that P(a|d)=0.5=P(a₂|d). Observing either aᵢ by itself may not provide information, however observing both may make the model more likely. score(m|a₁∧¬a₂) may be negative; having one true and not the other may be evidence against the model. The score may have a value such as score(m|¬a₁∧¬a₂)=0.2, which may be the same as the reward for when both may be true. Increasing the probability that both may be true, while keeping the marginals on each variable the same, may lead to increasing the probability that both may be false. If the values are selected carefully and increasing the score of ¬a₁∧¬a₂ is not desirable, then the scores of each aᵢ may be increased.

In another embodiment, a different semantics may. Suppose that using reward(a₁|m), reward(a₂|m) may provide a new model m₁. The reward may be rewardₘ₁(a₁∧a₂|m), and may not be comparing the conjunction to the default, but to m₁. The conjunction may be increased in m by some specified weight, and the other combinations of truth values a₁ and a₂ may be decreased in the same or a similar proportion. This may be a similar model as would be recovered by a logistic regression model with weights for the conjunction, as further described herein. In the embodiment, the score may be

score(m|a₁∧a₂)=reward(a₁|m)+reward(a₂|m)+reward(a₁∧a₂|m)

In the embodiment, the score(a₁|m) may not be equal to reward(a₁|m), but the embodiment may take into account the reward of a₁∧a₂. The reward(a₁|m) may be the value used for computing a score (e.g., any score) that may be incompatible with the exceptional conjunction a₁∧a₂, such as score(m|a₁∧¬a₂).

In some examples described herein, score(m|a₁∧a₂) may be score(m|a₁∧a₂)=0.2, may be expected and score(m|¬a₁∧¬a₂)=−0.0941, and may be the same as other combinations of negations of both attributes (e.g., as long as there is at least one negation). score(m|¬a₁)=may be score(m|¬a₁)=0.01317, which may be more than the reward that occurs if the conjunction was not also rewarded.

For example, suppose that using just reward(a₁|m), reward(a₂|m) gives a new model m₁. reward(a₁∧a₂|m) may not be comparing the conjunction to the default, but to m₁. The conjunction may be increased by m and the other combinations of truth values a₁ and a₂ may be decreased by the same or similar proportion. For example, the following may be considered:

reward(a₁∧a₂|m)=w₃

Then, the following may occur:

P(a₁∧a₂|m)=10^{w}^{₃}*P(a₁∧a₂|d)

P(a₁∧¬a₂|m)=c*P(¬a₁∧¬a₂|d)

P(¬a₁∧a₂|m)=c*P(¬a₁∧¬a₂|d)

P(¬a₁∧¬a₂|m)=c*P(¬a₁∧¬a₂|d) Equation(4)

These may sum to 1 such that c may be computed (e.g., assuming a₁ and a₂ may be independent in the default):

$$c = \frac{1 - {10}^{{w}_{3}} * P ( {α}_{1} \vert d ) * P ( {α}_{2} \vert d )}{1 - P ( {α}_{1} \vert d ) * P ( {α}_{2} \vert d )} \qquad Equation ( 5 )$$

This may be like Equation (3) but with the conjunction having the reward. The scores of the others may be decreased by log c. d may be sequentially updated to m₁ using the attributes (e.g., the single attributes), and then to update m₁ to m using the formula above. For example:

| | |
|---|---|
| reward(α₁ \| m) = w₁ | P(α₁ \| d) = p₁ |
| reward(α₂ \| m) = w₂ | P(α₂ \| d) = p₂ |
| reward(α₁ \hat{ } α₂ \| m) = w₃ | |

We will define m₁ by:

| | |
|---|---|
| reward(α₁ \| m₁) = w₁ | P(α₁ \| d) = p₁ |
| reward(α₂ \| m₁) = w₂ | P(α₂ \| d) = p₂ |

The formula in Equation (4) may be used using reward, (a₁∧a₂|m), such that m₁ may be used instead of d as the reference.

For score(m|a₁∧¬a₂), a₁∧¬a₂ may be treated as a single proposition.

$$\begin{matrix}{score}_{d} ( m \vert {α}_{1} ∧ ¬ {α}_{2} ) = log \frac{P ( {α}_{1} ∧ ¬ {α}_{2} \vert m )}{P ( {α}_{1} ∧ ¬ {α}_{2} \vert d )} \\ = log ( \frac{P ( {α}_{1} ∧ ¬ {α}_{2} \vert m )}{P ( {α}_{1} ∧ ¬ {α}_{2} \vert {m}_{1} )} * \frac{P ( {α}_{1} ∧ ¬ {α}_{2} \vert {m}_{1} )}{P ( {α}_{1} ∧ ¬ {α}_{2} \vert d )} ) \\ = log \frac{P ( {α}_{1} ∧ ¬ {α}_{2} \vert m )}{P ( {α}_{1} ∧ ¬ {α}_{2} \vert {m}_{1} )} + log \frac{P ( {α}_{1} ∧ ¬ {α}_{2} \vert {m}_{1} )}{P ( {α}_{1} ∧ ¬ {α}_{2} \vert d )} \\ = log \frac{1 - {10}^{{w}_{3}} * {p}_{1} * {p}_{2}}{1 - {p}_{1} * {p}_{2}} + {w}_{1} + log \frac{1 - {10}^{{w}_{2}} {p}_{2}}{1 - {p}_{2}}\end{matrix}$$

where the left side may be Equation (5) and the right two conjunctions may be the same as before (without the conjunction). The other cases where both a₁ and a₂ may be assigned truth values may be the same or similar as the independent cases (without w₃), but with an extra terms added for the assignments inconsistent with a₁∧a₂:

$$\begin{matrix}score ( m \vert {α}_{1} ∧ {α}_{2} ) = {w}_{1} + {w}_{2} + {w}_{3} \\ score ( m \vert {α}_{1} ∧ ¬ {α}_{2} ) = {w}_{1} + log \frac{1 - {10}^{{w}_{2}} {p}_{2}}{1 - {p}_{2}} + log \frac{1 - {10}^{{w}_{3}} * {p}_{1} * {p}_{2}}{1 - {p}_{1} * {p}_{2}} \\ score ( m \vert ¬ {α}_{1} ∧ {α}_{2} ) = log \frac{1 - {10}^{{w}_{1}} {p}_{1}}{1 - {p}_{1}} + {w}_{2} + log \frac{1 - {10}^{{w}_{3}} * {p}_{1} * {p}_{2}}{1 - {p}_{1} * {p}_{2}} \\ score ( m \vert ¬ {α}_{1} ∧ ¬ {α}_{2} ) = log \frac{1 - {10}^{{w}_{1}} {p}_{1}}{1 - {p}_{1}} + log \frac{1 - {10}^{{w}_{2}} {p}_{2}}{1 - p 2} + log \frac{1 - {10}^{{w}_{3}} * {p}_{1} * {p}_{2}}{1 - {p}_{1} * {p}_{2}}\end{matrix}$$

Consider the case where, for example, only at may be observed. The following may be used

P(a₁|·)=P(a₁∧a₂|·)+P(a₁∧¬a₂|·)

as long as they · may be replaced consistently. In the following s may be the right side of Equation (6):

$$\begin{matrix}reward ( m \vert {α}_{1} ) = log \frac{P ( {α}_{1} \vert m )}{P ( {α}_{1} \vert d )} \\ = log \frac{P ( {α}_{1} ∧ {α}_{2} \vert m ) + P ( {α}_{1} ∧ ¬ {α}_{2} \vert m )}{P ( {α}_{1} \vert d )} \\ = log \frac{P ( {α}_{1} ∧ {α}_{2} \vert d ) * {10}^{{w}_{1} + {w}_{2} + {w}_{3}} + P ( {α}_{1} ∧ ¬ {α}_{2} \vert d ) * {10}^{s}}{P ( {α}_{1} \vert d )} \\ = log \frac{P ( {α}_{1} \vert d ) * P ( {α}_{2} \vert d ) * {10}^{{w}_{1} + {w}_{2} + {w}_{3}} + P ( {α}_{1} \vert d ) * P ( ¬ {α}_{2} \vert d ) * {10}^{s}}{P ( {α}_{1} \vert d )} \\ = log P ( {α}_{2} \vert d ) * {10}^{{w}_{1} + {w}_{2} + {w}_{3}} + P ( ¬ {α}_{2} \vert d ) * {10}^{s} \\ = log ( {p}_{2} * {10}^{{w}_{1} + {w}_{2} + {w}_{3}} + ( 1 - {p}_{2} ) * {10}^{s} ) \\ = log {10}^{{w}_{1}} * ( {p}_{2} * {10}^{{w}_{2} + {w}_{3}} + ( 1 - {p}_{2} ) * {10}^{log \frac{1 - {p}_{2} * {10}^{{w}_{2}}}{1 - p 2} + log \frac{1 - {10}^{{w}_{3} * {p}_{1} * {p}_{2}}}{1 - {p}_{1} * {p}_{2}}} ) \\ = {w}_{1} + log ( {p}_{2} * {10}^{{w}_{2} + {w}_{3}} + ( 1 - {p}_{2} ) * \frac{1 - {p}_{2} * {10}^{{w}_{2}}}{1 - p 2} * \frac{1 - {10}^{{w}_{3}} * {p}_{1} * {p}_{2}}{1 - {p}_{1} * {p}_{2}} ) \\ = {w}_{1} + log ( {p}_{2} * {10}^{{w}_{2}} * {10}^{{w}_{3}} + ( 1 - {p}_{2} * {10}^{{w}_{2}} ) * \frac{1 - {10}^{{w}_{3}} * {p}_{1} * {p}_{2}}{1 - {p}_{1} * {p}_{2}} )\end{matrix} \qquad Equation ( 6 )$$

The term inside the log on right side may be a linear interpolation between 10^{w}^{₃} and the value of Equation (5), where the interpolation may be governed by p₂*10^{w}^{₂}.

For the other cases, a₁ may be any formula, and then a₂ may be the conjunction of the unobserved propositions that make up the conjunction that may be exceptional.

In another embodiment, it may be possible to specify one or more (e.g., all but 1) of the combinations of truth values: reward(a₁∧a₂|m), reward(a₁∧¬a₂|m) and reward(¬a₁∧a₂|m).

In another embodiment, conditional statement may be used. This may be achieved, for example, by using the context of the rewards. For example, the following may make the context explicit:
reward(a₁|m, c)
reward(a₂|m, at A c)
reward(a₂|m, ¬a₁∧c)

This may follow the idea of belief networks (e.g., Bayesian networks), where a₁ may be a parent of a₂. This may provide desirable properties in that the numbers may be as interpretable as for the non-conjunctive case for the cases where values for both a₁ and a₂ may be observed.

For example, in the landslide domain, different weights may be used for the trigger when the propensity may be present, and when it may be absent (e.g., the propensity becomes part of the context for the trigger).

There may be issued to be addressed that may arise because it may be asymmetric with respect to a₁ and a₂. For example, if only the conjunction needs to be rewarded, then it may not treat them symmetrically. The reward for a₁ may be assessed when a₂ may not be observed, and the reward for a₂ may be assessed in one or more (e.g., each) of the conditions for the values for a₁. The score for a₁ without a₂ being observed may be available (e.g., directly available) from the model, whereas the score for a₂ without a₁ being observed may be inferred.

Interaction with Aristotelian definitions may be provided. A class C may be defined in the Aristotelian way in terms of a conjunction of attributes:

p₁=v₁,p₂=v₂, . . . ,pₖ=vₖ

For example, object x may be in class C may be the equivalent to the conjunction of triples:

[symbol]

x,p₁,v₁ [symbol] ∧ [symbol] x,p₂,v₂ [symbol] ∧ . . . ∧ [symbol] x,pₖ,vₖ

[symbol]

It may be assumed that the properties may be ordered such that the domain of each property comes before the property. For example, the class may be defined by p₁=v₁∧ . . . ∧pᵢ₋₁=vᵢ₋₁ may be a subclass of domain(pᵢ)). Assuming false∧x may be false even if x may be undefined, this conjunction may be defined (e.g., may always be well defined).

For example, a granite may be defined as:

(x,type,granite)≡(x,genetic,igneious)∧(x,|fesic_status,felsic)∧(x,source,intrusive)∧(x,texture,phaneritic)

In instances, this may be treated as a conjunction. For example, observing (x, type, granite) may be equivalent to a conjunction of the proprieties defining a granite.

In models, the definition may be as a conjunction as disclosed herein. For example, if granite may have a (positive) reward, then the conjunction may have that reward. Any sibling and cousin of granite (which may differ in at least one value and may not be a granite) may have a negative reward. A more general instance (e.g., providing a subset of the attributes) may have a positive reward, as it may be possible that it is a granite. The reward may be in proportion to the probability that it may be a granite. Related concepts may have a positive reward by adding that conjunction to the rewards. For example, a reward may be provided for a component (e.g., each component) of a definition and a reward for more general conjunctions (such as [symbol] x, genetic, igneious [symbol] ∧ [symbol] x, fesic_status, felsic [symbol] ∧ [symbol] x, texture, phaneritic [symbol] . The reward for granite may them be distributed among them subsets of attributes.

Parts and aggregations may be provided. For example, rewards may interact with parts. In an example, a part may be identifiable in the instance. The existence of the part may be observable and may be observed to be false. This may occur in mineral assemblages and may be applicable when the grouping depends on the model.

Rewards may be propagated. Additional hypotheses may be considered, such as whether a part exists and whether a may be true in those parts.

It may be assumed that in the background, the probability of an attribute a may not depend on whether the part exists or not. It may be assumed that the model may not specify what happens to a when the part does not exist, and that it may use the same as in the background. For example, it may be assumed that P(a|m∧¬p)=P(a|d).

With these assumptions, attribute a and part p may be provided for as follows:
reward(p|m, c) for a part p
reward(¬p|m, c) for a part p
reward(a|m, p∧c)—notice how the part may join the context
reward(¬a|m, p∧c)

As disclosed herein, from the first two P(p|m) and P(p|d) may be computed (e.g., as long as they are both not zero; in that case P(p|m) or P(p|d) may need to be specified). And from the second two P(a|p∧m) and P(a|p∧d) may be computed.

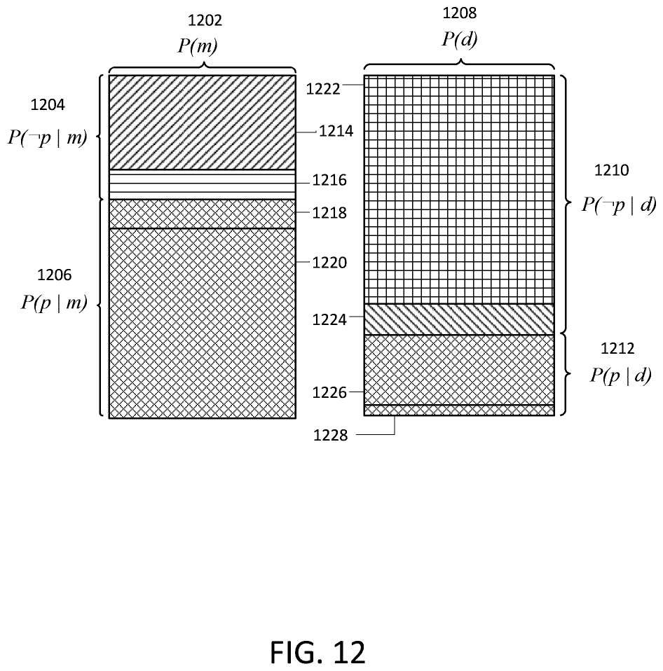

FIG. 12 shows an example depiction of a probability of an attribute, where the presence of the attribute may indicate a weak positive and an absence of the attribute may indicate a weak negative. At 1206, P(p|m)=0.6. At 1212, P(p|d)=0.3. As shown in FIG. 12, P(a|p A m)=0.9 and P(a|d)=0.2. m∧a may be true at 1220 and/or 1216. d A a may be true at 1224 and/or 1228. Part p may be true in the areas at 1218, 1220, 1226, and/or 1228. Part p may be false in the areas at 1214, 1216, 1222, and/or 1224.

If a∧p may be observed, the reward may be as follows:

reward(a∧p|m,c)=reward(a|m,p∧c)+reward(p|m,c)

Model propagation may be provided. In an example embodiment, a model may have parts by an instance may not have parts.

If a may be observed (e.g., so the instance may not be divided into parts), then the following may be provided:

$$\begin{matrix}\frac{P ( α \vert m ∧ c )}{P ( α \vert d ∧ c )} = \frac{P ( α ∧ p \vert m ∧ c ) + P ( α ∧ ¬ p \vert m ∧ c )}{P ( α \vert d ∧ c )} \\ = \frac{\begin{matrix}P ( α \vert p ∧ m ∧ c ) * P ( p \vert m ∧ c ) + \\ P ( α \vert ¬ p ∧ m ∧ c ) * P ( ¬ p \vert m ∧ c )\end{matrix}}{P ( α \vert d ∧ c )} \\ = \frac{\begin{matrix}P ( α \vert p ∧ m ∧ c ) * P ( p \vert m ∧ c ) + \\ P ( α \vert d ∧ c ) * ( 1 - P ( p \vert m ∧ c ) )\end{matrix}}{P ( α \vert d ∧ c )} \\ = \frac{P ( α \vert p ∧ m ∧ c )}{P ( α \vert d ∧ c )} * P ( p \vert m ∧ c ) + ( 1 - P ( p \vert m ∧ c ) )\end{matrix}$$

This may be a linear interpolation between

$$\frac{P ( α \vert p ∧ m ∧ c )}{P ( α \vert d ∧ c )}$$

and 1. For example, a linear interpolation between x and y may be x*p+y*(1−p) for 0≤p≤1.

The rewards may be as follows:

$$\begin{matrix}reward ( α \vert m , c ) = log \frac{P ( α \vert m ∧ c )}{P ( α \vert d ∧ c )} \\ = log ( {10}^{reward ( α \vert m , p ∧ c )} * \\ P ( p \vert m ∧ c ) + ( 1 - P ( p \vert m ∧ c ) ) )\end{matrix}$$

This may not be simplified further. And this value may be (e.g., may always be) of the same sign, but closer to 0 than reward(a|m, p∧c).

As disclosed herein, in an example, if a may have observed in the context of p, then the reward may be reward(a|m, p∧c)=log 0.9/0.2=0.635, which may be added to the reward of p. If just a may have been observed (e.g., not in any part), the reward may be as follows:

$$\begin{matrix}reward ( α \vert m , c ) = log \frac{P ( α \vert m ∧ c )}{P ( α \vert d ∧ c )} \\ = log ( 0. 9 / 0.2 * 0.6 + 0.4 ) \\ = 0.49 1\end{matrix}$$

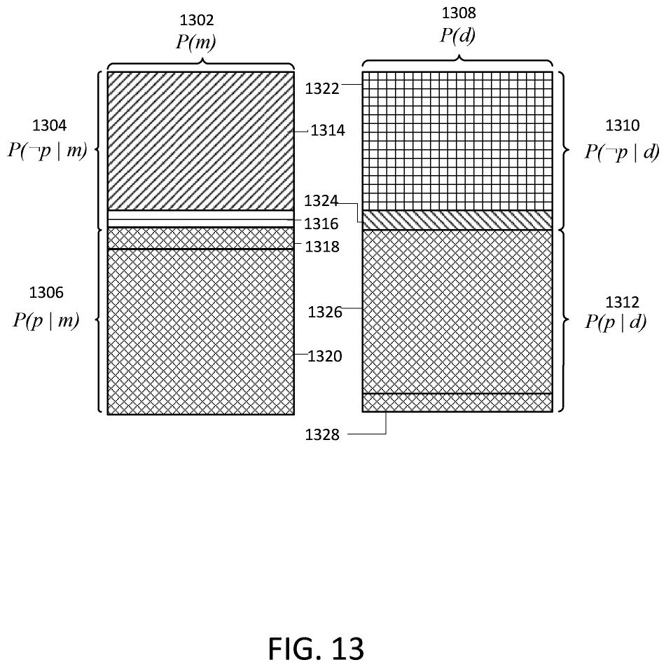

FIG. 13 shows an example depiction of a probability of an attribute, where the presence of the attribute may indicate a weak positive and an absence of the attribute may indicate a weak negative. As shown in FIG. 13, a part may have zero reward, and may not be diagnostic. The probability of the part may be middling, but a may be diagnostic (e.g., very diagnostic).

For example, the rewards may be reward(p|m, c)=0 and the probability may be P(p|m)=0.5. In this case, it may be that reward(p|¬m, c)=0 and P(p|d)=0.5. The reward may be reward(a|m, p∧c)=1, such that a may be diagnostic (e.g., very diagnostic) of the part. If the instances may not have any part, then the following may be derived:

$$\begin{matrix}reward ( α \vert m , c ) = log ( 10 * 0.5 + 0.5 ) \\ = 0.74\end{matrix}$$

Observing a may eliminate the areas at 1310, 1312, 1314, and/or 1318. This may make m more likely.

The reward may be reward(a|m, p∧c)=2, such that a may even be more diagnostic of the part. If the instances did not have any part, then the following may be derived:

$$\begin{matrix}reward ( α \vert m , c ) = log ( 100 * 0.5 + 0.5 ) \\ = 1.703\end{matrix}$$

Existence uncertainty may be provided. The existence of some part (e.g., some mineralization) may be evidence for or against a model. There may be multiple parts in an instance that may possibly correspond to the part that may be hypothesized to exist in the model. To provide an explainable output, it may be desirable to identify which parts may correspond.

For positive reward cases, the embodiments may allow such statements as follows:
M1: there exists a large, bright room. This is true if a large bight room is identified.

For negative rewards, there may be one or more possible statements (e.g., two possible statements):
M2: there usually exists a room that is not green. This is true if a non-green room is identified. The green rooms are essentially irrelevant.
M3: no room is green. (e.g., There usually does not exists a green room.) The existence of a green room is contra-evidence for this model. In this case, green rooms may be looked for.

In an example, the first and second of these (M1 and M2) may be addressed.

For example, the following may be known:
max(P(x), P(y))≤P(x∨y)≤P(x)+P(y)≤1

An extreme (e.g., each extreme) may be possible. For example, P(x∨y)=max(P(x), P(y) when one of x and y implies the other, and P(x∨y)=P(x)+P(y) when x are y are mutually exclusive.

An example may have 2 parts in an instance p₁ and p₂ and the model may have a₁ . . . aₖ in part p with some reward. The probability of the match (which may correspond to P(a∧(p₁∨p₂)) may then be maxᵢ(P(a∧pᵢ)), which may provide the following:

$$reward ( α \vert m , c ) = {max}_{i} reward ( α ∧ {p}_{i} \vert m , c )$$

This may provide a role assignment, which may specify the argmax (e.g., which i gives the max value).

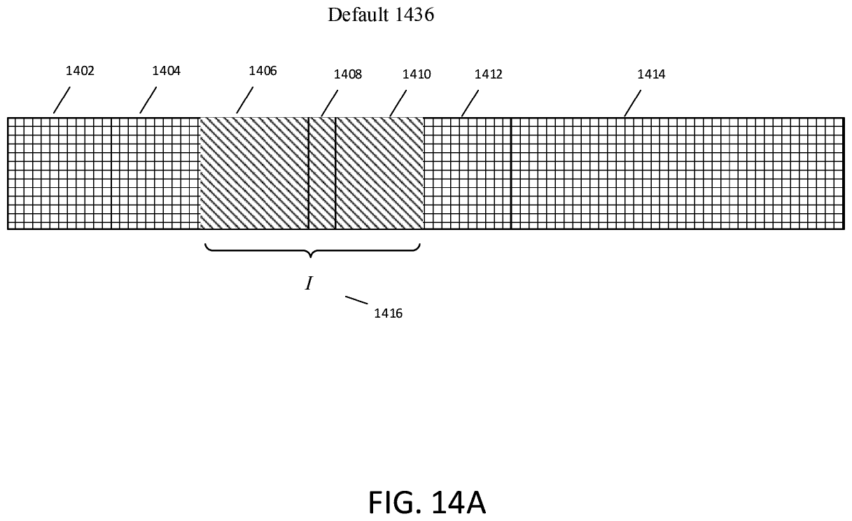

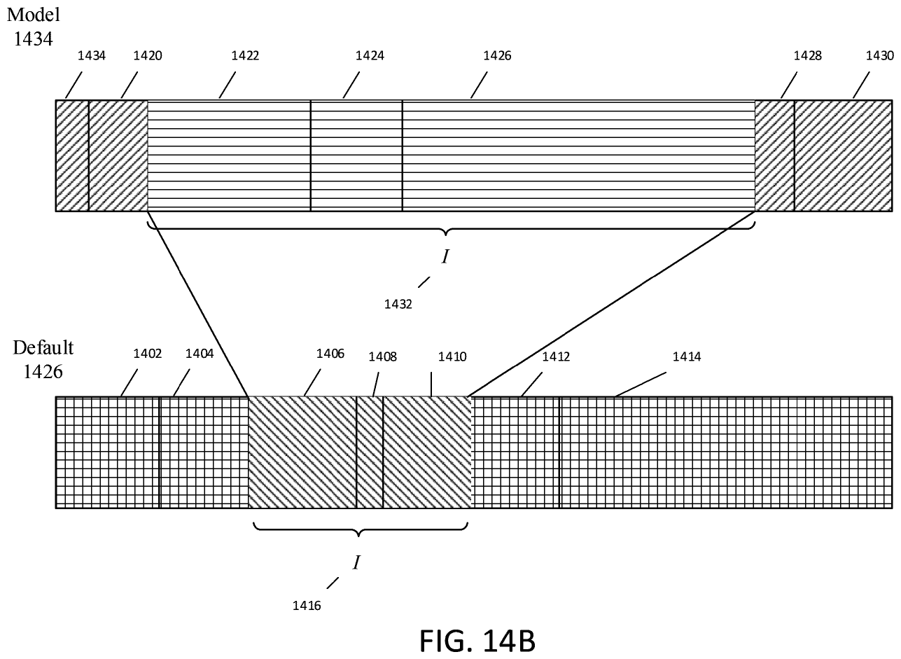

Interval reasoning may be provided. FIG. 14A shows an example depiction of default that may be used for interval reasoning. FIG. 14B shows an example depiction of a model that may be used for interval reasoning.

In an example, a range of a property may be numeric (e.g., a single number). This may occur for time (e.g., both short-term and geological time), weight, slope, height, and/or the like. Something more sophisticated may be used for multi-dimensional variables such as color or shape when they may be more than a few discrete values.

An interval may be squashed into the range [0,1], where the length of an interval may correspond to its probability.

FIG. 14A shows an example depiction of default that may be used for interval reasoning. As shown in FIG. 14A, a distribution of a real-valued property, may be divided into 7 regions, where an interval I is shown at 1416. The 7 regions may be 1402, 1404, 1406, 1408, 1410, 1412, and 1414. The regions may be in some hierarchical structure. The default may be default 1436.

FIG. 14B shows an example depiction of a model that may be used for interval reasoning. As shown in FIG. 14B, a distribution of a real-valued property, may be divided into 7 regions, where an interval I is shown at 1432. The 7 regions may be 1418, 1420, 1422, 1424, 1426, 1428, and 1430. The regions may be in some hierarchical structure. Model 1434 may specify the interval I at 1432, which may be bigger than the interval I at 1416. Then everything else may stretch or shrink in proportion. For example, when I expands, the intervals in I may expand by the same amount (e.g., 1422, 1424, 1426), and the intervals outside of I may shrink by the same amount (e.g., 1434, 1428, 1430).

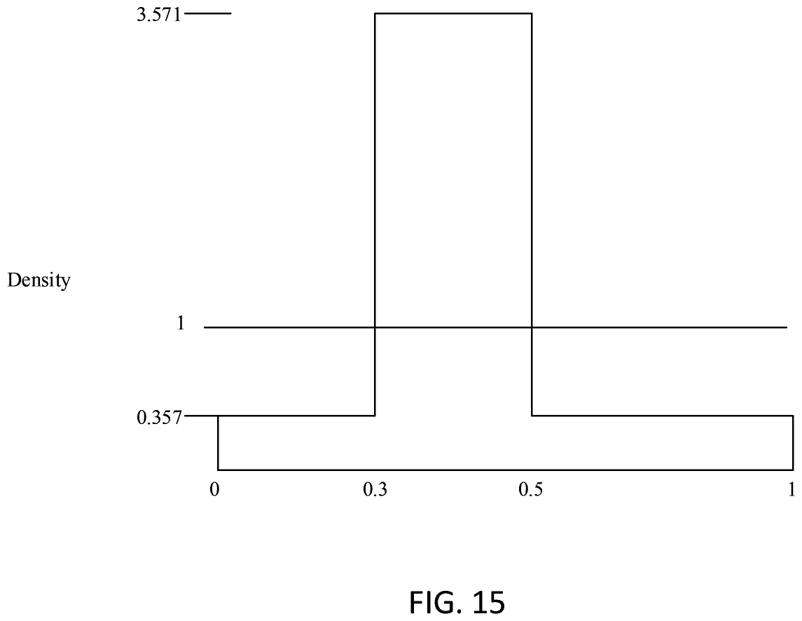

FIG. 15 shows an example depiction of a density function for one or more of the embodiments. For example, FIG. 15 may represent change in intervals shown in FIGS. 14A and 14B as a product of the default interval and a probability density function. In a probability density function the x-axis is the default interval, and the area under the curve is 1. This density function may specify what the default may be multiplied by to get the model. The default may correspond to the density function that may be the constant function with value 1 in range [0,1].

In the example density function, the top area may be the range of the value that is more likely given the model, and the lower area may be the range of values that are less likely given the model. The model probability may be obtained by multiplying the default probability by the density function. In this model, the density of the interval [0.3,0.5] may be 10 times the other values.

The two numbers that may be multiplied may be the height of the density function in the interval I:

$$k = \frac{P ( I \vert m ∧ c )}{P ( I \vert d ∧ c )}$$

and the height of the density function outside of the interval I may be provided as follows:

$$r = \frac{P ( ¬ I \vert m ∧ c )}{P ( ¬ I \vert d ∧ c )} = \frac{1 - P ( I \vert m ∧ c )}{1 - P ( I \vert d ∧ c )}$$

The interval [I0, I1] that is modified by the model may be known. Then the probability in the model may be specified by one or more of the following:
P(I|m∧c), how likely the interval may be in the model. This may be the area under the curve for the interval in the density function.
k, the ratio of how much more likely I may be in the model than in the default. This may be constrained by:

$$0 \leq k \leq \frac{1}{P ( I \vert d ∧ c )}$$

r, the ratio of how much more likely intervals outside of I may be in the model than in the default. This may be constrained by the fact that probabilities are in the range [0,1].
k/r this may be the ratio of the heights in the density function. This may have the advantage that the ratio may be unconstrained (it may take a nonnegative value (e.g., any nonnegative value)).

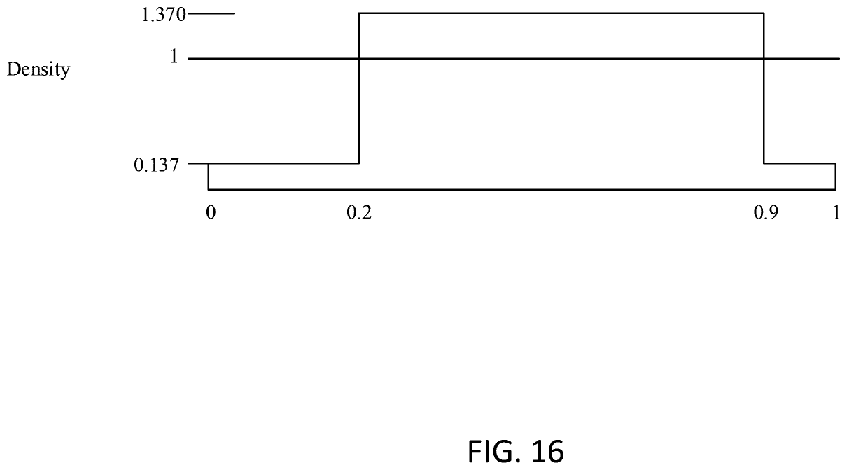

FIG. 16 shows another example depiction of a density function for one or more of the embodiments. In this model, the density of the interval [0.2,0.9] may be 10 times the other values. For the interval [0.2,0.9], the reward may be at most 1/0.7≈1.43.

Interval instance, single exceptional model interval may be provided. An instance may be scored that may be specified by an interval (e.g., as opposed to a point observation). In the instance, interval J may be observed, and the model may have I specified. J may be partitioned into J∩I, the overlap (or set intersection) between J and I, and J/I, the part of J outside of I. The reward may be computed using the following:

$$\begin{matrix}\frac{P ( J \vert m ∧ c )}{P ( J \vert d ∧ c )} = \frac{P ( J ∩ I \vert m ∧ c ) + P ( J ∖ I \vert m ∧ c )}{P ( J \vert d ∧ c )} \\ = \frac{P ( J ∩ I \vert m ∧ c )}{P ( J \vert d ∧ c )} + \frac{P ( J ∖ I \vert m ∧ c )}{P ( J \vert d ∧ c )} \\ = \frac{P ( J ∩ I \vert m ∧ c )}{P ( J ∩ I \vert d ∧ c )} * \frac{P ( J ∩ I \vert d ∧ c )}{P ( J \vert d ∧ c )} + \\ \frac{P ( J ∖ I \vert m ∧ c )}{P ( J ∖ I \vert d ∧ c )} * \frac{P ( J ∖ I \vert d ∧ c )}{P ( J \vert d ∧ c )} \\ = k * \frac{P ( J ∩ I \vert d ∧ c )}{P ( J \vert d ∧ c )} + r * \frac{P ( J ∖ I \vert d ∧ c )}{P ( J \vert d ∧ c )}\end{matrix}$$

where k and r may be provided as described herein. This may be a linear interpolation of k and r where the weights may be given by the default model.

Reasoning with more of the distribution specified may be provided. The embodiment may allow for many rewards or probabilities to be specified while the others may be able to grow or shrink so as to satisfy probabilities and to maintain one or more ratios.

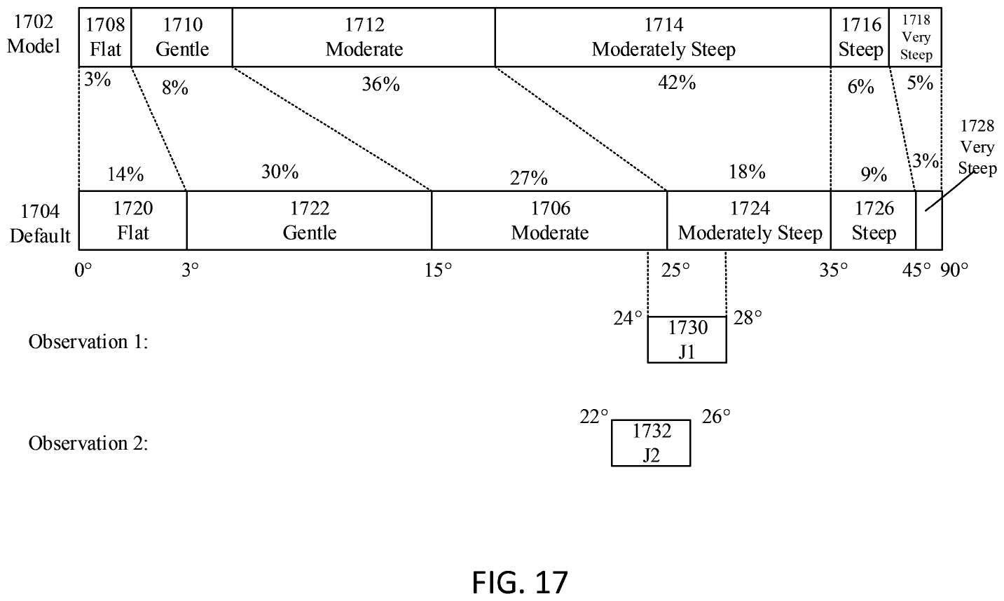

FIG. 17 shows an example depiction of a model and default for an example slope range. FIG. 17 shows a slope for a model at 1702 and a default at 1704. Smaller ranges of slope (e.g., if moderate at 1706 was divided into smaller subdivisions), may be expanded or contracted in proportion to the range specified. The rewards or probabilities of model 1702 may be provided at 1708, 1710, 1712, 1714, 1716, and 1718. 1708 may indicate a flat slope (0-3 percent grade) with a 3% probability. 1710 may indicate a gentle slope (3-15 percent grade) with an 8% probability. 1712 may indicate a moderate slope (15-25 percent grade) with a 36% probability. 1714 may indicate a moderately steep slope (25-35 percent grade) with a 42% probability. 1716 may indicate a steep slope (35-45 percent grade) with a 6% probability. 1718 may indicate a very steep slope (45-90 percent grade) with a 5% probability.

The rewards or probabilities of default 1704 may be provided at 1720, 1722, 1706, 1724, and 1726. 1720 may indicate a flat slope (0-3 percent grade) with a 14% probability. 1722 may indicate a gentle slope (3-15 percent grade) with a 30% probability. 1706 may indicate a moderate slope (15-25 percent grade) with a 27% probability. 1724 may indicate a moderately steep slope (25-35 percent grade) with a 18% probability. 1726 may indicate a steep slope (35-45 percent grade) with a 9% probability. 1718 may indicate a very steep slope (45-90 percent grade) with a 3% probability.

Given the default at 1704, the model at 1702 may specify five of the rewards or probabilities (as there are six ranges). The other one may be computed because the sum of the possible slopes may be one. The example may ignore overhangs, with slopes greater that 90. This may be done for simplicity in demonstrating the embodiments described herein as overhangs may be complicated considering there may be 3 or more slopes at any location that has an overhang.

The rewards for observations may be computed as described herein, where the observations may be considered as disjoint unions of smaller intervals. The observed ranges in may not be contiguous. For example, it may be observed that something happened on a Tuesday in some April, which may be discontiguous intervals. Although this is not explored in this example, discontiguous intervals may be implemented and/or used by the embodiments disclosed herein.

If the qualitative intervals may be observed, then the following may be provided:

$$reward ( gentle \vert m ) = log \frac{P ( gentle \vert m )}{P ( gentle \vert d )} = log 0.08 / 0.30 = - 0.5 \; 740 reward ( moderate \vert m ) = log \frac{P ( moderate \vert m )}{P ( moderate \vert d )} = log 0.36 / 0.27 = 0.1 \; 368 reward ( moderate\_steep \vert m ) = log \frac{P ( moderate\_steep \vert m )}{P ( moderate\_steep \vert d )} = log 0.42 / 0.18 = 0.3680$$

If a different interval may be observed, a case analysis of how that interval overlaps with the specified intervals may be performed. For example, consider interval J1 at 1732 in FIG. 17. This may be seen as the union of two intervals, 24-25 degrees and 25-28 degrees. The first may be 1/10 of the moderate range and may grow like the moderate, and the second may be 3/10 of the moderately steep and may grow like moderately steep. For example:

$$\begin{matrix}reward ( J 1 \vert m ) = log \frac{P ( J 1 \vert m )}{P ( J 1 \vert d )} \\ = log \frac{1 / 10 * 0.36 + 3 / 10 * 0.42}{1 / 10 * 0.27 + 3 / 10 * 0.18} \\ = log \frac{0.162}{0.081} \\ = log 2. \\ = 0.301\end{matrix}$$

Similarly, for observation 2 at 1732:

$$\begin{matrix}reward ( J 2 \vert m ) = log \frac{P ( J 2 \vert m )}{P ( J 2 \vert d )} \\ = log \frac{3 / 10 * 0.36 + 1 / 10 * 0.42}{3 / 10 * 0.27 + 1 / 10 * 0.18} \\ = log \frac{0.15}{0.099} \\ = log 1.51515 \\ = 0.18046\end{matrix}$$

As shown above, these may be between the rewards of moderate and moderately steep, with J1 more like moderately-steep and J2 more like moderate.

The model may specify intervals I₁ . . . Iₙ as exceptional (and may include the other intervals such that I₁∪ . . . ∪Iₙ covers the whole range, and Iⱼ∩Iₖ={ } for j≠k). J may be the interval or set of intervals in the observation. Then the following may be provided:

$$\begin{matrix}reward ( J \vert m ) = log \frac{P ( J \vert m )}{P ( J \vert d )} \\ = log {\sum }_{i} \frac{P ( J ∩ {I}_{i} \vert m )}{P ( J \vert d )} \\ = log {\sum }_{i} \frac{P ( J ∩ {I}_{i} \vert m )}{P ( J ∩ {I}_{i} \vert d )} * \frac{P ( J ∩ {I}_{i} \vert d )}{P ( J \vert d )}\end{matrix}$$

Point observations may be provided. If the observation in an instance may be a point, then if the point may be interior to an interval (e.g., not on a boundary) that may have been specified in the model, the reward may be used for that interval. There may be a number of ways to handle a point that is on a boundary.

For example, the modeler may be forced to specify to which side a boundary interval is. This may be done by agreeing to a convention that an interval from i to j means {x|i<x≤j}, which may be written as the interval (i, j], or means {x|i≤x<j} which may be written as the interval [i, j).

As another example, it may be assumed that a point p means the interval [p−ϵ, p+ϵ] for some small value ϵ (where ϵ may be small enough to stay in an interval; this may give the same result as taking the limit as ϵ approaches 0).

In FIG. 17, an observation of 25 degrees, may be the observation of the interval (24,26), which may have the following reward:

$$\begin{matrix}reward ( Model \vert ( 24 , 26 ) ) = log \frac{P ( ( 24 , 26 ) \vert Model )}{P ( ( 24 , 26 ) \vert Model )} \\ = log \frac{1 / 10 * 0.36 + 1 / 10 * 0.42}{1 / 10 * 0.27 + 1 / 10 * 0.18} \\ = log \frac{0.078}{0.045} \\ = log 1.7333 \\ = 0.23888\end{matrix}$$

In another example, it may be assumed that the interval around the point observation may be equal in the default probability space. In this case, the reward may be the log of the average of the probability ratios of the two intervals, moderate and moderately steep. For example, the rewards of the two intervals may be as follows:

$$\begin{matrix}reward ( Model \vert ( 24 , 26 ) ) = log \frac{36 / 27 + 42 / 18}{2} \\ = 0.26324\end{matrix}$$

These may have a difference that may be subtle. For example, it may be difficult for an expert to ascertain whether the error may be in the probability estimate or may be in the actual measurement. It may make a difference (e.g., a big difference) when a large interval with a low probability may be next to a small interval with a much larger probability. For example, in geological time there are very old-time scales that include many years.

As described herein, the embodiments may provide clear semantics that may allow a correct answer to be calculated according to the semantics. The inputs and the outputs may be interpreted consistently. The rewards may be learned from data. Reward for absent may not be inferred from reward for present. What numbers to specify may be designed such that it may make sense to experts. A basic matching program may be provided. Instances and/or existing models may not need to be changed as the program may add the rewards in recursive descent through models and instances. English terms and/or labels may be translated into to rewards. Existential uncertainty may be provided. For example, properties of zones that may or may not exist. Interval uncertainty, such as time, may be provided. Models may be compared with models.

Relationships to logistic regression may be provided. This model may be similar to a logistic regression model with a number of properties.

In an example embodiment, missing information may be modeled. For example, for an attribute (e.g., each attribute) a there may be a weight for the presence a and a weight for the absense of a (e.g., a weight for a and a weight for ¬a). Neither weight may be used if a may not be observed. This may allow both the model and logistic regression to learn the probability of the default (e.g., when nothing may be specified); it may be the sigmoid of the bias (the parameter may not be multiplied by a proposition (e.g., any proposition)).

A base-10 may be used instead of base-e to aid in interpretability. A weight (e.g., each weight) may be explained and interpreted when comparing the background. In some cases, such as simple cases, they may be interpreted as log-odds as described herein. Both may be interpreted when there may be more complex formulas (e.g., conjunctions with weights). To change the base, the weights may be multiplied by a constant as described herein.

If a logistic regression model may be used, the logistic regression may be enhanced for intervals, parts, and the like. And a logistic regression model may be supported.

A derivation of logistic regression may be provided. For example, In may be the natural logarithm (base e), and it may be assumed none of the probabilities may be zero:

$$\begin{matrix}P ( m \vert α ) = \frac{P ( m ∧ α )}{P ( α )} \\ = \frac{P ( m ∧ α )}{P ( m ∧ α ) + P ( ¬ m ∧ α )} \\ = \frac{1}{1 + \frac{P ( ¬ m ∧ α )}{P ( m ∧ α )}} \\ = \frac{1}{1 + {e}^{l n \frac{P ( ¬ m ∧ α )}{P ( m ∧ α )}}} \\ = \frac{1}{1 + {e}^{- l n \frac{P ( m ∧ α )}{P ( ¬ m ∧ α )}}} \\ = sigmoid ( ln odds ( m \vert α ) )\end{matrix}$$

where sigmoid(x)=1/(1+e^{-x}), and

$$odds ( m \vert α ) = \frac{P ( m ∧ α )}{P ( ¬ m ∧ α )} .$$

For example sigmoid may be connected (e.g., deeply connected) with probability (e.g., conditional probability). If the odds may be a product then the log-odds may be a sum. Logistic regression may be seen as a way to find a product decomposition of a conditional probability.

If the observations may be a₁ . . . aₖ, and the aᵢ may be independent given m (which may have the assumption made above before logical formulae and conjunction were introduced), then the following may be provided:

$$( P ( m \vert α ) = sigmoid ( ln odds ( m ) + {\sum }_{i = 1}^{k} ln odds ( m \vert {α}_{i} ) )$$

which may be similar to Equation (2).

Base 10 and base e may be a product difference:

10^{x}=e^{ln10}^{^{x}}=e^{x*ln10}≈e^{2.3*x}

Converting from base 10 to base e may be performed by multiplying by ln10≈2.3. Converting from base e to base 10 may be done by dividing by ln10.

The formalism chosen may have been done so to estimate the probability of a model in comparison with a default that a comparison with what happens when the model may not be true. It may be difficult to learn the weights for the logistic regression when random sampling may not occur. For example, the model may be compared to a default distribution in some part (e.g., small part) of the world by sampling locally, but global sampling may assist in estimating the odds.

Default probabilities may be provided which may use partial knowledge, missing attributes heterogenous models, observations, and/or the like.

For many cases, when building models of the world, a small part of the world may be seen. It may be possible say what happens when the model holds (e.g., P(a|m) for an attribute a), but may not be used to determine the global average P(a), which may be used to compute the probability of m given a is observed, namely P(m|a). This may use a complete and covering set of hypotheses, or the ability to sample P(a) directly. P(m|a) may not be computed to compare different models, but may use the ratio between them

When models and observations may be heterogenous, may make predictions on different observations, it may not be possible to simply compute the ratios.

These problems may be solved by choosing a default distribution and specifying how the models may differ from the default. The posterior ratio between the model and the default may allow us to compare models without computing the probability of the attributes, and may also allow for heterogenous observations, where missing attributes may be interpreted as meaning the probability may be the same as the default.

Heterogenous models and observations may be provided. Many domains (e.g., real domains) may be characterized by heterogeneous observations at multiple levels of abstraction (in terms of more and less general terms) and detail (in terms of parts and subparts). Many domains (e.g., real domains) may be characterized by multiple hypotheses/models that may be made by different people at multiple levels of abstraction and detail and may not cover one or more possibilities (e.g., all possibilities). Many domains (e.g., real domains) may be characterized by a lack of access to the whole universe of interest and so may not be able to sample to determine the prior probabilities of features (or what may be referred to as the “partion function” in machine learning). For an observations/model pair (e.g., each observations/model pair), the model may have one or more missing attributes (which may be part of the model, but may not be observed) and missing data may not be missing at random, and the model may not predict a value for the attribute.

The use of default probabilities, where a model (e.g., each model) may be calibrated with respect to a default distribution where one or more attributes (e.g., all attributes) may be missing, may allow for a solution.

An ontology may be provided. An ontology may be concepts that are relevant to a topic, domain of discourse, an area of interest, and/or the like. For example, an ontology may be provided for information technology, computer languages, a branch of science, medicine, law, and/or other expert domains.

In an example, an ontology may be provided for an apartment to generate probabilistic reasoning for an apartment search. For example, the ontology may be used by one or more servers to generate a probabilistic reasoning that may aid a user in searching for an apartment. While this example may be done for an apartment search, other domains of known may be used. For example, an ontology may be used to generate probabilistic reasoning for medicine, healthcare, real estate, insurance markets, mining, mineral discovery, law, finance, computer security, geological hazard discovery, and/or the like.

A classification of rooms may have a number of considerations. A room may or may not have a role. For example, when comparing the role of a bedroom versus a living room, the living room may be used as a bedroom, and a bedroom may be used as a TV room or study. When looking at a prospective apartment, the current role may not be the role a user may use for a room. Presumably someone may be interested in the future role they may use a room for rather than the current role. Some rooms may be designed as specialty rooms, such as bathrooms or kitchens. In those case, it may be assumed that “kitchen” may mean a room with plumbing for a kitchen rather than the role it may be used for.

A room may often be defined (e.g., well defined). For example, in a living—dining room division, there may be a wall with a door between them or they may be open to each other. If they may be open to each other, some may say they may be different rooms, because they are logically separated, and other might say they may be one room. There may be a continuum of how closed off from each other they are. A bedroom may be difficult to define. A definition may be a bedroom as a room that may be made private. But a bedroom may not be limited to that definition. For example, if you remove the door from a bedroom it may not stop the room from being a bedroom. However, if a user were to see an apartment advertised with bedrooms that were open to the rest of the apartment, that person may feel that the advertising was misleading

In an example embodiment, the physical aspects of the space may be separated from the role. And a probabilistic model may be used to predict future roles. People may also be allowed to make up roles.

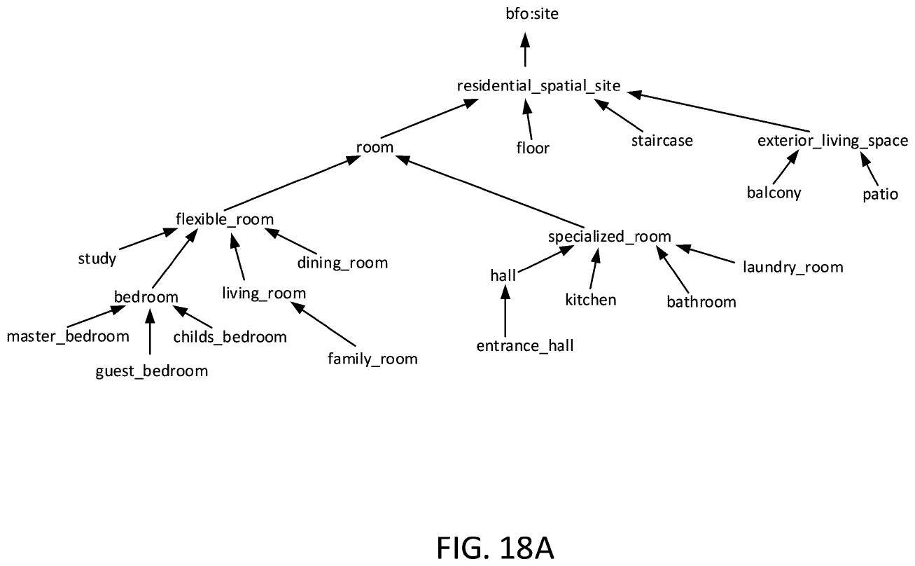

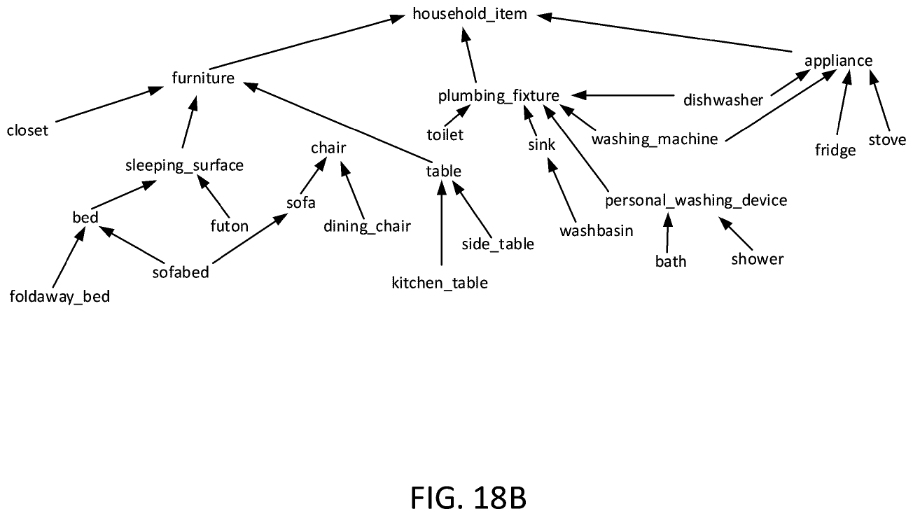

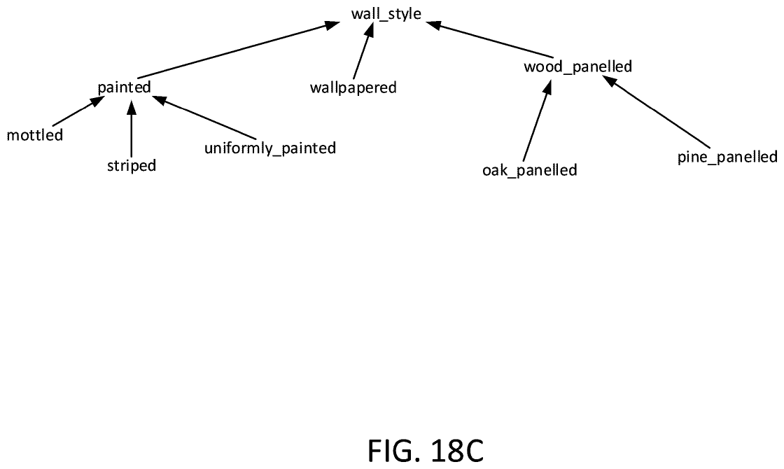

FIGS. 18A-C depict example depictions of one or more ontologies. For example, the one or more ontologies shown in FIGS. 18A-C may be used to described room, household-items, and/or and wall-style. FIG. 18A may depict an example ontology for a room. FIG. 18B may depict an example ontology for a household item. FIG. 18C may depict an example ontology for a wall style. The one or more ontologies shown in FIGS. 18A-C may provide a hierarchy for rooms, household items, and/or wall styles.

Using FIG. 18A, an example hierarchy may be as follows:
room=residential_spatial_site & enclosed_by=walls & size=human_sized specialized_room=room & is_specialty_room=true
kitchen=specialized_room & contains=sink & contains=stove & contains=fridge
bedroom=room & is_specialty_room=false & made_private=true

An ontology may be provided by color. The ontology for color may be defined by someone who knows about color, such as an expert about human perception, someone who worked at a paint store, and/or the like. Color may be defined in terms of 3 dimensions: hue, saturation and brightness. The brightness may depend on the ambient light and may not be a property of the wall paint. The (e.g., daytime) brightness may be a separate property of rooms and apartments. Grey may be considered a hue.

For the hue, it may be assumed that the colors may be the values of a hue property. For example, it may be a functional property. Hue may be provided for as follows: range hue [red, orange, yellow, green, blue, indigo, violet, grey]

Similarly, the saturation may be as values. Saturation may be a continuum, a 2 dimensional, one or more ranges and the like. Rang saturation may be provided for as follows:
range saturation [deep_color, rich_color, light_color, pale_color]

Example classes of colors may be defined as follows:
Pale_pink=Color & hue=red & saturation=pale_color
Pink=Color & hue=red & saturation in [pale_color, light_color]
Red=Color & hue=red
Rich_Red=Color & hue=red & saturation=rich_color
Deep_red Color & hue=red & saturation=deep_color

In an example, for the (daytime) brightness of rooms, the following may be used:
range brightness={sunny, bright, shaded, dark}

where (e.g., for the Northern Hemisphere) sunny may means south-facing and unshaded, bright may be East or West facing, shaded may be North facing or otherwise in shade, and dark may mean that it may be darker than would be expected from a North-facing window (e.g., because of small windows or because there is restricted natural lighting).

An example instance of an apartment using one or more ontologies may be provided as follows:
Apartment34
   size=large
   contains_room
      type bedroom
      size small
      has_wall_style mottled
   contains_room
      type bathroom
      has_wall_style wallpapered

In the example instance above, the apartment may contain 2 rooms (e.g., at least 2 rooms), one of which may be a small mottled bedroom, and the other of which may be a wallpapered bathroom.

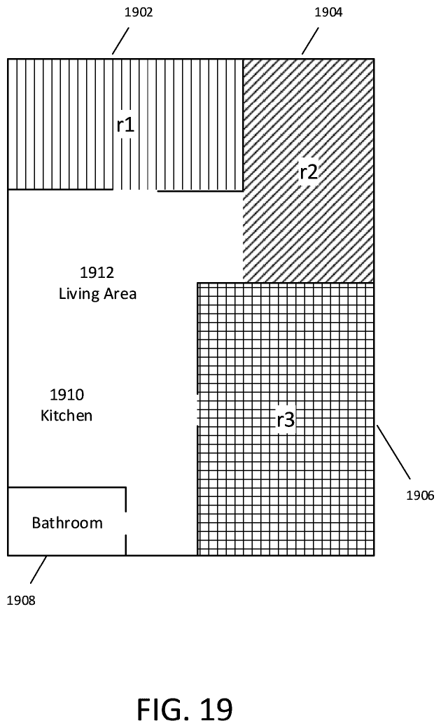

FIG. 19 may depict an example instance of a model apartment that may use one or more ontologies. As shown in FIG. 19, may have a room that contains both a kitchen and a living room. There may be a question whether the kitchen and the living room may be considered separate rooms. As shown in FIG. 19, the example apartment may have a bathroom at 1908, a kitchen at 1910, a living room at 1912, bedroom r1 at 1902, bedroom r2 at 1903, and bedroom r3 at 1906. The instance of the apartment in FIG. 19 may be provided as follows:
Apartment77
   size=large
   contains_room r1
      type bedroom
      color orange
   contains_room r2
      type bedroom
      size small
      color pink
      brightness bright
   contains_room r3
      type bedroom
      size large
      color green
      brightness shaded
   contains_room br
      type bathroom
   contains_roon mr
      type kitchen
      type living room
      brightness sunny
   contains_room other absent

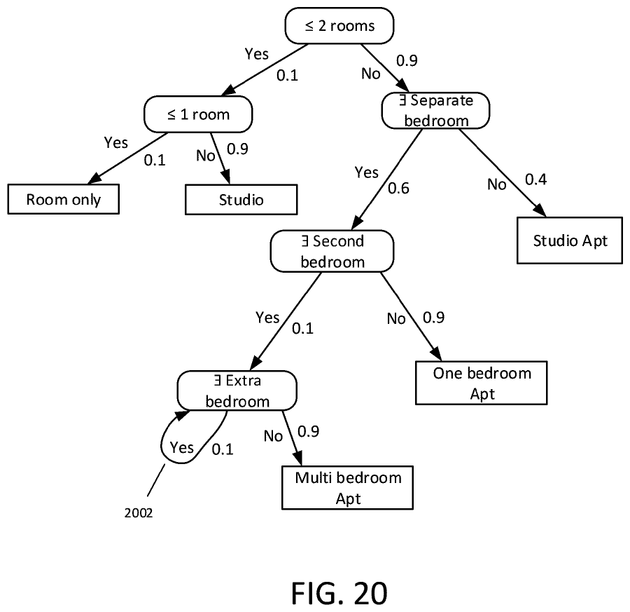

FIG. 20 may depict an example default or background for a room. For example, FIG. 20 may show a default for the existence of rooms of certain types, such as bedrooms. As shown in FIG. 20, at 2002, the loop under “there exists another bedroom” may mean that there may not be a bound to the number of bedrooms, but there may be an exponential distribution on the number of bedrooms beyond 2. In the default, the other probabilities may be independent of the number of rooms.

For the color of the walls, there may be two dimensions as described herein. As these may be functional properties, a distribution may be chosen. These colors of rooms may be assumed to be independent in the default. But there may be alternatives to the assumption of independence. For example, a color theme may be chosen, and the colors may depend on the theme. As another example, the color may depend on the type of the room.

In a default, hue and saturation may be provided as follows:

Hue:
red: 0.25, orange: 0.1, yellow: 0.1, green: 0.2, blue: 0.2, indigo: 0.05, violet: 0.05, grey: 0.05

Saturation:
deep_colour: 0.1, rich_colour: 0.1, light_colour: 0.7, pale_colour: 0.1

So, for example, it may be assumed that a room (e.g., all rooms) may have a color. The probability of the color given the default may be determined. For example, the probability for pink given the default may be as follows:

$$\begin{matrix}P ( pink \vert d ) = \begin{matrix}P ( Colour \& hue = red \& saturationin \{ pale\_colour , \\ light\_colour \} )\end{matrix} \\ = 0.25 * 0.8 \\ = 0.2\end{matrix}$$

The brightness (e.g., daytime brightness) may depend on the window size and direction and whether there may be a clear view. A distribution may be:
sunny: 0.2, bright: 0.5, shaded: 0.3, dark: 0.1

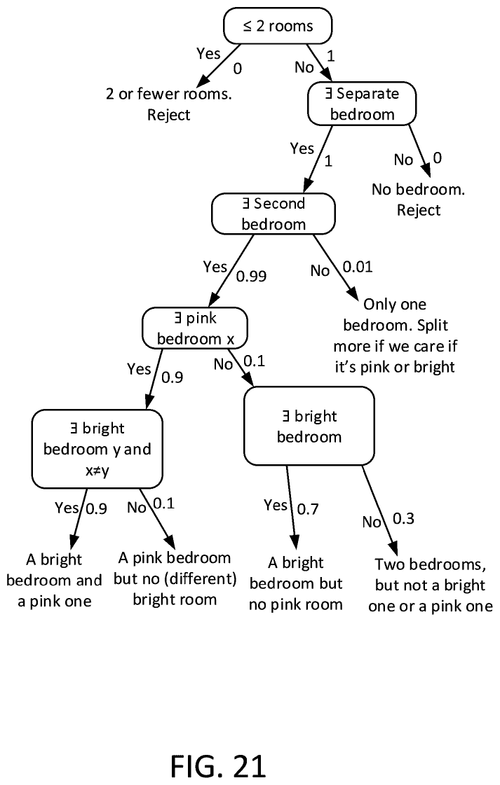

A model may be provided. A model may specify how it may differ from a default (e.g., the background). FIG. 21 may depict how an example model may differ from a default.

In an example, a model may be labeled Model01. The model may be a model for a two-bedroom apartment.

In an example, a user may want a two-bedroom apartment. The user may want at least one bedroom. And the user may prefer a second bedroom. The user may prefer that one bedroom is sunny, and a different bedroom is pink. An example model may specify how what the user wants may differ from the default. And the model may omit one or more thing that the user may not care about.

In the default, which may consider that there may be multiple bedrooms of which one or more may be pink:

$$P ( ∃ pink bedroom \vert d ) = 0.9 * 0.6 * ( 1 - ( 1 - 0.1 ) * ( 1 - 0.08 ) / ( 1 - 0.1 + 0.1 * 0.08 ) ) = 0.047577$$

The left two products may be reading down the tree of FIG. 21, and the right may be from the derivation of P(pink|d) as described herein.

The following may be provided:

$$\begin{matrix}reward ( ∃ pink bedroom \vert m , \{ \} ) = log \frac{P ( ∃ pink bedroom \vert m )}{P ( ∃ pink bedroom \vert d )} \\ \approx \frac{1 * 1 * ( 0.99 * 0.9 * 0.01 )}{0.047577} \\ = log 18.94 \\ 1.277\end{matrix}$$

where the approximation may be because it may not have been model what happens when there may not be a second bedroom.

The reward may be as follows:

$$\begin{matrix}\begin{matrix}reward ( ∃ x : pink ( x ) ∧ bedroom ( x ) ∧ \\ ∃ y : bright ( y ) ∧ bedroom ( y ) ∧ x \neq y \vert m , \{ \} )\end{matrix} = log \frac{\begin{matrix}P ( ∃ x : pink ( x ) ∧ \\ bedroom ( x ) ∧ \\ ∃ y : bright ( y ) ∧ \\ bedroom ( y ) ∧ x \neq y \vert m )\end{matrix}}{\begin{matrix}P ( ∃ x : pink ( x ) ∧ \\ bedroom ( x ) ∧ \\ ∃ y : bright ( y ) ∧ \\ bedroom ( y ) ∧ x \neq y \vert d )\end{matrix}} \\ = \frac{1 * 1 * 0.99 * 0.9 * 0.9}{\begin{matrix}0.9 * 0.6 * 0.1 * ( 1 - \\ ( 1 - 0.1 ) * {( 1 - 0.08 )}^{2} / ( 1 - \\ 0.1 + 0.1 * 0.08 ) ) * \\ ( 1 - ( 1 - 0.1 ) * ( 1 - 0.5 )\end{matrix}} \\ = log 161.05 \\ = 2.207\end{matrix}$$

where the numerator may be from following the branches for FIG. 20.

The reward for the existence of a bright room and a separate pink room may be as follows:
exists pink bedroom=+1
exists sunny bedroom=+1.5

Expectation over an unknown number of objects may be provided. It may be known that there are k objects, and the probability of some property may be true is p for each object, then the probability that there exists an object with that property may be:

P(∃x:p(x))=1−(1−P)^{k}

which may be 1 minus the probability that the property may be false for one or more objects (e.g., all objects). p may be used for both the probability and the property, but it should be clear which is which from the context.

It may be known that there are (at least) k objects, and for each number of objects, the probability that there exists another object (e.g., an extra object) may be e. For example, the existence of another room in FIG. 20 may fits this pattern.

the number of extra objects may be summed over (where i may be the number of extra objects); eⁱ(1−e) may be the probability that there may be i extra objects, and there may exist an object with k+i objects (e.g., i extra objects) with probability (1−(1−p)^{k+i}) The following may be provided:

$$\begin{matrix}P ( ∃ ( \geq 1 ) x : p ( x ) \vert ∃ ( \geq k ) x ) = {\sum }_{i = 0}^{∞} {e}^{i} ( 1 - e ) ( 1 - {( 1 - p )}^{k + i} ) \\ = ( 1 - e ) ( {\sum }_{i = 0}^{∞} {e}^{i} - {( 1 - p )}^{k} {\sum }_{i = 0}^{∞} {( e ( 1 - p ) )}^{i} ) \\ = \begin{matrix}( 1 - e ) / ( 1 - e ) - \\ ( 1 - e ) {( 1 - p )}^{k} / ( 1 - e ( 1 - p ) )\end{matrix} \\ = 1 - ( 1 - e ) {( 1 - p )}^{k} / ( 1 - e + ep )\end{matrix}$$

$$Because s = {\sum }_{i = 0}^{∞} {x}^{i} = 1 + xs = 1 / ( 1 - x ) .$$

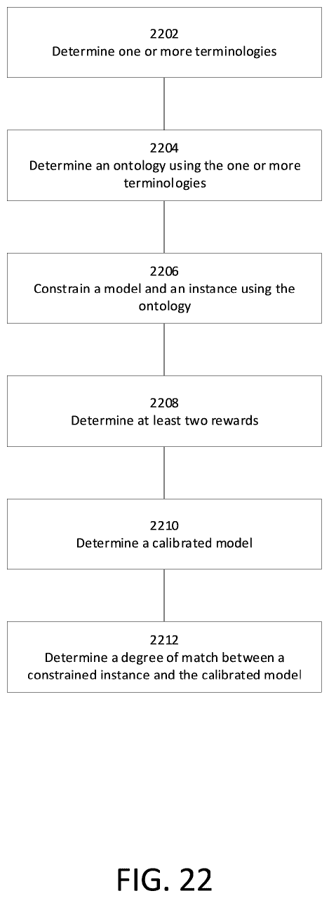

FIG. 22 may depict an example flow chart of a process for expressing a diagnosticity of an attribute in a conceptual model. At 2202 one or more terminologies may be determined. A terminology may assist in describing an attribute. For example, the terminology for an attribute may be “color blue” for a color attribute of a model room. A terminology may be considered a taxonomy. For example, the terminology may be a system for naming, defining, and/or classifying groups on the basis of attributes.

For example, a terminology may be provided for geologists, which may use scientific vocabulary to describe their exploration targets and the environments they occur in. The words in these vocabularies may occur within sometimes complex taxonomies, such as the taxonomy of rocks, the taxonomy of minerals, and the taxonomy of geological time, and the like.

At 2204, an ontology may be determined using the one or more terminologies. An ontology may be a domain ontology. The ontology may help describe a concept relevant to a topic, a domain of discourse, an area of interest, and/or an area of expertise. For example, a terminology may be provided for geologists, which may use scientific vocabulary to describe their exploration targets and the environments they occur in. The words or terms in these vocabularies may occur within one or more taxonomies (e.g., one or more terminologies), such as the taxonomy of rocks, the taxonomy of minerals, and the taxonomy of geological time, to mention only a few. An ontology may incorporate these taxonomies into a reasoning. For example, the ontology may indicate that that basalt is a volcanic rock, but granite is not.

At 2206, a model and an instance may be constrained, for example, using an ontology. An ontology may be defined using one or more terminologies in the domain of expertise. For example, a terminology may be provided for geologists, which may define scientific vocabulary to describe their exploration targets and the environments they occur in. The words or terms in these vocabularies may occur within one or more taxonomies (e.g., one or more terminologies), such as the taxonomy of rocks, the taxonomy of minerals, and the taxonomy of geological time, to mention only a few. An ontology may incorporate these taxonomies into a reasoning. For example, the ontology may indicate that that basalt is a volcanic rock, but granite is not.

A constrained model and a constrained instance may be determined by constraining a model and an instance using the ontology. For example, the model may be constrained by defining the model by expressing one or more model attribute using the ontology. As an example, a model that may be used by a geologist may be constrained by the ontology used by the geologist. The instances may be constrained in a similar manner.

At 2208, at least two rewards are determined. A reward may be determined as described herein. A reward may be a function of four arguments: d, a, m and c. For example, the reward of attribute a may be determined given model m and context c, with the default d. When c may be empty (or the proposition may be true) the last argument may sometimes be omitted. When d is empty, it may be understood by context and it may also be omitted. The reward may be calculated using the following equation:

$${reward}_{d} ( α \vert m , c ) = log \frac{P ( α \vert m ∧ c )}{P ( α \vert d ∧ c )}$$

The reward (a|m, c) may tell us how much more likely a may be, in context c, given the model was true, than it was in the background.

At 2210, a calibrated model may be determined. The model may be determined as described herein. The calibrated model may be determined by calibrating the constrained model to a default model using a terminology from the one or more terminologies to express a first reward and a second reward. The first reward and/or the second rewards may be a frequency of the attribute in the model, a frequency of the attribute in the default model, a diagnosticity of a presence of the attribute, or a diagnosticity of an absence of the attribute. The first reward may be different from the second reward.

At 2212, a degree of match between a constrained instance and the calibrated model may be determined. The degree of match may indicate how the constrained instance may relate to the calibrated model. For example, the degree of match may indicate how useful the model may be, a probability of the model, and degree of accuracy of a model, a degree of accuracy of the model predicting the instance, and the like.

A device for expressing a diagnosticity of an attribute in a conceptual model may be provided. The device may be the device at 141 with respect to FIG. 1 The device may comprise a memory and a processor. The processor may be configured to perform a number of actions. One or more terminologies in a domain of expertise for expressing one or more attributes may be determined. An ontology may be determined using the one or more terminologies in the domain of expertise. A constrained model and a constrained instance may be determined by constraining a model and an instance using the ontology. A calibrated model may be determined by calibrating the constrained model to a default model using a terminology from the one or more terminologies to express a first reward and a second reward. A degree of match between the constrained instance and the calibrated model may be determined. A probabilistic rationale may be generated using the degree of match. The probabilistic rationale may explain how the degree of match was reached.

An ontology may be determined using the one or more terminologies in the domain of expertise by determining one or more terms of the one or more terminologies. One or more links between the one or more terms of the one or more terminologies may be determined. Use of the terms (e.g., the one or more terms) may be constrained to express a possible description of the attribute.

In an example, a number of actions may be performed to determine the constrained model and the constrained instance using the ontology. A description of the model may be generated using the one or more links between the terms of the one or more terminologies. A description of the instance may be generated using the one or more links between the terms of the one or more terminologies.

In an example, a number of actions may be performed to determine the calibrated model by calibrating the constrained model to a default model using a terminology from the one or more terminologies to express a first reward and a second reward. The first reward and/or the second rewards may be a frequency of the attribute in the model, a frequency of the attribute in the default model, a diagnosticity of a presence of the attribute, or a diagnosticity of an absence of the attribute. The first reward may be different from the second reward. The frequency of the attribute in the model, the frequency of the attribute in the default model, the diagnosticity of the presence of the attribute, and the diagnosticity of the absence of the attribute may be calculated as described herein (e.g., FIGS. 2-14B).

The first and second reward may be used to calculate a third and fourth rewards. For example, the first reward may be the frequency of the attribute in the model. The second reward may be the diagnosticity of the presence of the attribute. As described herein, the frequency of the attribute in the model and the diagnosticity of the presence of the attribute in the model may be used to derive the frequency of the attribute in the default model and/or the diagnosticity of the absence of the attribute.

The attribute may be a property-value pair. The domain of expertise may be a medical diagnosis domain, a mineral exploration domain, an insurance market domain, a financial domain, a legal domain, a natural hazard risk mitigation domain, and/or the like.

The default model may comprise a defined distribution over one or more property values. The model may describe the attribute that should be expected to be true when the instance matches the model. The model may comprise a sequence attributes with a qualitative measure of prediction confidence. The instance may comprise a tree of attributes defined by the one or more terminologies in the domain of expertise. The instance may comprise a sequence of attributes defined by the one or more terminologies in the domain of expertise.

A method implemented in a device for expressing a diagnosticity of an attribute in a conceptual model may be provided. One or more terminologies in a domain of expertise for expressing one or more attributes may be determined. An ontology may be determined using the one or more terminologies in the domain of expertise. A constrained model and a constrained instance may be determined by constraining a model and an instance using the ontology. A calibrated model may be determined by calibrating the constrained model to a default model using a terminology from the one or more terminologies to express a first reward and a second reward. A degree of match may be determined between the constrained instance and the calibrated model.

A computer readable medium having computer executable instructions stored therein may be provided. The computer executable instructions may comprise a number of actions. For example, one or more terminologies in a domain of expertise for expressing one or more attributes may be determined. An ontology may be determined using the one or more terminologies in the domain of expertise. A constrained model and a constrained instance may be determined by constraining a model and an instance using the ontology. A calibrated model may be determined by calibrating the constrained model to a default model using a terminology from the one or more terminologies to express a first reward and a second reward. A degree of match may be determined between the constrained instance and the calibrated model.

As described herein, a device may be provided for expressing a diagnosticity of an attribute in a conceptual model. One or more terminologies may be determined in a domain of expertise for expressing one or more attributes. An ontology may be determined using the one or more terminologies in the domain of expertise. A constrained model and a constrained instance may be determined by constraining a model and an instance using the ontology. A calibrated model may be determined by calibrating the constrained model to a default model using a terminology from the one or more terminologies to express a first reward and a second reward. A degree of match may be determined between the constrained instance and the calibrated model.

A probabilistic rationale may be generated using the degree of match. The probabilistic rationale explaining how the degree of match was reached.

An ontology may be determined using the one or more terminologies in the domain of expertise by determining terms of the one or more terminologies and determining one or more links between the terms of the one or more terminologies. The one or more links between the terms of the one or more terminologies may be determined by constraining a use of the terms to express a possible description of the attribute.

A constrained model and/or constrained instance may be determined, for example, using the ontology. A description of the model may be generated using the one or more links between the terms of the one or more terminologies. A description of the instance may be generated using the one or more links between the terms of the one or more terminologies.

The first reward may be a frequency of the attribute in the model, a frequency of the attribute in the default model, a diagnosticity of a presence of the attribute, or a diagnosticity of an absence of the attribute. The first reward may be different from the second reward, and the second reward may be the frequency of the attribute in the model, the frequency of the attribute in the default model, the diagnosticity of the presence of the attribute, or the diagnosticity of the absence of the attribute. A third reward and/or a fourth reward may be determined using the first reward and the second reward.

An attribute may be a property-value pair. A domain of expertise may be a medical diagnosis domain, a mineral exploration domain, a natural hazard risk mitigation domain, and/or the like.

A default model may comprise a defined distribution over one or more property values. A model may describe the attribute that may be expected to be true when the instance matches the model. A model may comprise a sequence attributes with a qualitative measure of prediction confidence.

An instance may comprise a tree of attributes defined by the one or more terminologies in the domain of expertise. An instance may comprise a sequence of attributes that may be defined by one or more terminologies in the domain of expertise.

Implementations of the described techniques may include hardware, a method or process, or computer software on a computer-accessible medium. For example, as disclosed herein, a device may be provided for expressing a diagnosticity of an attribute in a conceptual model. The device may include a memory, and a processor, the processor configured to perform a number of actions. One or more model attributes may be determined that may be relevant for a model. The model may be defined by expressing, for each model attribute in the one or more model attributes, at least two of a frequency of the model attribute in the model, a frequency of the model attribute in a default model, a diagnosticity of a presence of the model attribute, and a diagnosticity of an absence of the model attribute. An instance may be determined that may include one or more instance attributes, where an instance attribute in the one or more instance attributes may be assigned a positive diagnosticity when the instance attribute may be present and may be assigned a negative diagnosticity when the instance attribute may be absent. A predictive score for the instance may be determined by summing contributions made by the one or more instance attributes. An explanation associated with the predictive score may be determined for each model attribute in the one or more model attributes using the frequency of the model attribute in the model and the frequency of the model attribute in the default model.

The predictive score may indicate a predictability or likeliness of the model. The instance may be a first instance, the predictive score may be a first predictive score. A second instance may be determined a second predictive score may be determined. A comparative score may be determined using the first predictive score and the second predictive score. The comparative score may indicate whether the first instance or the second instance offers a better prediction.

The positive diagnosticity may be associated with a diagnosticity of the presence of a correlating model attribute from the one or more model attributes. The negative diagnosticity may be associated with a diagnosticity of the absence of a correlating model attribute from the one or more model attributes.

A prior score of the model may be determined by comparing a probability of the model to a default model. A posterior score may be determined for the model and the instance using the prior score and the predictive score.

As described herein, a device may be provided for expressing a probabilistic reasoning of an attribute in a conceptual model. The device may include a memory and a processor. The processor may be configured to perform a number of actions. A model attribute may be determined that may be relevant for a model. The model may be determined by expressing at least two of a frequency of the model attribute in the model, a frequency of the model attribute in a default model, a probabilistic reasoning of a presence of the model attribute, a probabilistic reasoning of an absence of the model attribute. An instance may be determined and may include at least an instance attribute that has a positive probabilistic reasoning or a negative probabilistic reasoning. A predictive score may be determined for the instance using a contribution made by the instance attribute. An explanation associated with the predictive score may be determined using the frequency of the model attribute in the model and the frequency of the model attribute in the default model.

The instance may be a first instance and the predictive score may be a first predictive score. A second instance may be determined. A second predictive score may be determined. A comparative score may be determined using the first predictive score and the second predictive score. The comparative score may indicate whether the first instance or the second instance offers a better prediction. The predictive score may indicate a predictability or likeliness of the model.

The positive probabilistic reasoning may be associated with the probabilistic reasoning of the presence of the model attribute. The negative probabilistic reasoning may be associated with the probabilistic reasoning of the absence of the model attribute.

A prior score of the model may be determined by comparing a probability of the model to a default model. A posterior score may be determined for the model and the instance using the prior score and the predictive score

As described herein, a method may be provided for expressing a probabilistic reasoning of an attribute in a conceptual model. The method may be performed by a device. A model attribute may be determined that may be relevant for a model. The model may be determined by expressing at least two of a frequency of the model attribute in the model, a frequency of the model attribute in a default model, a probabilistic reasoning of a presence of the model attribute, a probabilistic reasoning of an absence of the model attribute. An instance may be determined and may include at least an instance attribute that has a positive probabilistic reasoning or a negative probabilistic reasoning. A predictive score may be determined for the instance using a contribution made by the instance attribute. An explanation associated with the predictive score may be determined using the frequency of the model attribute in the model and the frequency of the model attribute in the default model.

The instance may be a first instance and the predictive score may be a first predictive score. A second instance may be determined. A second predictive score may be determined. A comparative score may be determined using the first predictive score and the second predictive score. The comparative score may indicate whether the first instance or the second instance offers a better prediction. The predictive score may indicate a predictability or likeliness of the model.

The positive probabilistic reasoning may be associated with the probabilistic reasoning of the presence of the model attribute. The negative probabilistic reasoning may be associated with the probabilistic reasoning of the absence of the model attribute.

A prior score of the model may be determined by comparing a probability of the model to a default model. A posterior score may be determined for the model and the instance using the prior score and the predictive score.

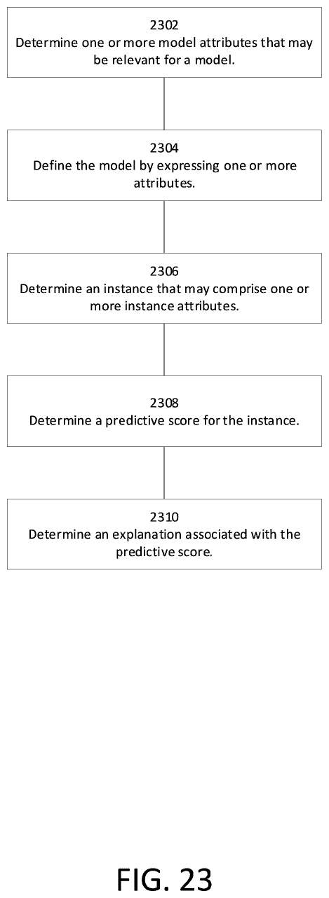

FIG. 23 depicts another example flow chart of a process for expressing a diagnosticity of an attribute in a conceptual model. The process may be carried a device that may comprise a memory and a processor. For example, the processor may be configured to the processor or a portion of the process shown in FIG. 23.

At 2302, one or more model attributes that may be relevant for a model may be determined.

At 2304, the model may be defined by expressing one or more attributes. For example, the model may be defined by expressing one or more attributes with their corresponding reward. The model may be defined by expressing one or more attributes using any of the methods described herein. For example, the model may comprise a sequence attributes with a qualitative measure of prediction confidence. The one or more attributes may be expressed as one or more terminologies in a domain of expertise. For example, an ontology may be determined and may be used to express the one or more attributes. And the one or more attributes and the ontology may be used to define the model.

A model with attributes may be used to provide probabilistic interpretation of scores. One or more values or numbers may be specified for an attribute. For example, two numbers may be specified for an attribute (e.g., each attribute) in a model; one number may be applied when the attribute is present in an instance of the model, and the other number may be when the attribute is absent. The rewards may be added to get a score (e.g., total score). In many cases, one of these may be small enough so that it may be effectively ignored, except for cases where it may be the differentiating attribute (in which case it may be a small ε value such as 0.001). If the model does not make a prediction about an attribute, that attribute may be ignored.

At 2306, an instance that may comprise one or more instance attributes may be determined. The instance may be determined as described herein. An instance may comprise a tree of attributes defined by the one or more terminologies in the domain of expertise. An instance may comprise a sequence of attributes that may be defined by one or more terminologies in the domain of expertise.

At 2308, a predictive score for the instance may be determined. The predictive score may indicate a predictability or likeliness of the model. A predictive score may be determined for the instance using a contribution made by the instance attribute. The score of a model, and for the reward of an attribute a given a model m in a context c (where the model may specify how the attributes of the instance update the score of the model) may be provide as follows:

$${score}_{d} ( m \vert c ) = {log}_{10} \frac{P ( m \vert c )}{P ( d \vert c )}$$

At 2310, an explanation associated with the predictive score may be determined. An explanation associated with the predictive score may be determined using the frequency of the model attribute in the model and the frequency of the model attribute in the default model.

A probability distribution may imply a probability of a hypothesis and a probability of evidence, however there may be cases where these may not be available, or there may be cases where more assumptions may be needed than may be reasonable. For example, the probability of a soil slide without an understanding of anything regarding the location is difficult to estimate and experts may be reluctant to try. In some embodiments, as described herein, there may not be a reliance on making global probability assumptions. For example, global probability assumptions may not be used to determine a probability. A probability ratio may be used. The probability ratio may allow for calibrating one or more (e.g., all) probabilities with respect to a default assignment of values to variables, and independence may be expressed using a ceteris paribus (e.g., everything else being equal) semantics. For example, embodiments described herein may allow for the expression of statements such as landslides are three times as likely on a steep slope than they are on a moderate slope. Such statements may be useful, explainable, and may be better suited to being transported from one location to another. And such statements may be used to provide predictions in a number of fields, such as the medical field, the product recommendation field, the geology field and the like. While examples provide examples from an application in landslide prediction, the embodiments may be applied to other fields to provide predictions.

Explainable models may be built. Models may be learned in one location and may be applied in others. This may be referred to as transportability of conditionals. For example, the transportability of conditionals may allow for observed features to be used to compare hypotheses that may be conditioned on the observation in a probabilistic framework.

The assignment of a value to a variable may be a proposition. The conjunction, negation or disjunction of propositions may also be a proposition.

In an example, a prediction of soil slides may be provided where the inputs may be slope, rock type, fire (e.g., number of years ago, or none), and logging (e.g., number of years ago, or none). A location with steep slope may be observed. The location may be observed with no fire in a recorded history, with an indication that it was clearcut 12 years ago, with an indication that it is on granite. A probability of a soil slide in that location may be predicted using:

P(soil slide|Slope=steep∧Fire=none∧Clearcut=12yearsago∧Rocktype=granite)

In the above equation, the description of the location is on the right-hand side of | and ∧ means “and”. In the above equation, random variables may start with upper case letters, and the lower case variant may be the proposition that the value of the variable is true (e.g., soil slide means Soil slide=true).

This may arguably be the appropriate causal direction, as a feature (e.g., each feature) on the right-hand side may have a causal effect on soil slides. The model may be transportable, explainable, and learnable. There may be other causal effects that may not be used in the modelling, which may vary from one location to another.

There may be one or more ways to represent a conditional probability, from tables to decisions trees to neural networks. There may be differences in model which may not occur for artificial domains with tables or trees, but may arises in applications where some generalization may be applied, or where one or more causes (e.g., all causes) may not be modeled.

A standard representation of a conditional probability may be logistic regression, which may be extended to the softmax for multi-valued variables. It may be typical to have a sigmoid or a softmax as the last layer of a neural network that makes probabilistic predictions. In some embodiments, a sigmoid may be used, which may be applicable for making predictions for Boolean features.

When H is Boolean (and h means H=true), P(h|e)=sigmoid (log odds(h|e)) where sigmoid(x) 1/(1+e^{-x}) and

$$odds ( h \vert e ) = \frac{P ( h ∧ e )}{P ( ¬ h ∧ e )} .$$

Logistic regression may make the independence assumption that

$$\frac{P ( h ∧ e )}{P ( ¬ h ∧ e )} ,$$

where e=e₁ . . . eₖ, decomposes into a product of terms, one for each eᵢ. Taking logs, the product may become a sum, to give the standard logistic regression formulation. A weight may have a meaning in terms of odds. For example, each of the weights may have a meaning in terms of odds, but may relies on assessing ratios involving P(h|¬e . . . ), which may be unknowable in the soil slide domain for locations that may not be known.

It may not be possible to assess the probability given the slope may not be steep, as it may depend on the distributions of slopes, and so the probability may not be directly transportable.

The weights of logistic regression may not be assessed. For example, people may rarely assess the weights of logistic regression directly. A similar problem may arise when learning a logistic regression model from data. The weights learned may depend on the data conditioned on, and it may be desirable to learn stable predictions. In an example, the training data may include a distribution of slopes, and the conditional probabilities may be sensitive to this distribution, which may not reflect the distribution in the location that the model may be applied to. A complete table may have a similar issue when variables being conditioned on may not include one more relevant variables (e.g., all relevant variables), and it may be rare for real world domains as the conditional may depend on distribution of the unmodelled variables. Modular representations like logistic regression may rely on comparing what happens when a feature is true to compared to when the feature is false. The weight associated with Slope=steep may reflect the effect of observing this proposition is true as opposed to observing it is false. This may be problematic when an observation is false may covers too many cases for it to be meaningful or stable to transportation.

It may be easier to assess a comparison than it is to assess any of the probabilities directly. For example, assessing the value for:

$$\frac{P ( soil slide \vert slope = steep ∧ x )}{P ( soil slide \vert slope = moderate ∧ x )}$$

where x may be any other value. This may be an assessment of how much more (or less) likely is a soil slide when the slope is steep, compared to when it is moderate. This may be something that experts may be willing to assess and may be measurable. The statement that this is true for any x may be considered a ceteris paribus—everything else being equal—assumption.

Instead of comparing a feature value with its negation or assessing the probability directly, it may be comparing it to a well-defined default. This may be weaker information than is provided by the conditional probability or conditional odds, and may provide weaker results. However, the information may be easier to acquire and explain, it may be transportable (e.g., but may require one number for calibration in a new location for each conditional probability to extract the conditional distribution), and the conclusions may be useful (e.g., even without calibration) in that allow for a comparison of hypotheses in useful ways.

In an embodiment, it may be assumed that the probability is defined on variables X₁ . . . Xₙ, where a variable has a range, a disjoint and covering set of values the variable may take on. For the sake of simplicity, that the range of each variable may be discrete.

The assignment of a value to a variable may be a proposition. The conjunction, negation or disjunction of propositions may also be a proposition.

The term “instance” may be used for a set of observed values v=v₁ . . . vₙ where vᵢ may be the instance's value for variable Xᵢ, leaving the variable implicit. Note that v, by itself, is the tuple, which may be used as an instance description.

An instance (e.g., each instance) may be compared to a well-defined default. A default may be denoted as d=d₁ . . . dₙ, where dᵢ may be a default value for variable Xᵢ. d is the tuple of assignments to the corresponding properties. A fixed default may be assumed. Although a changing default may be use in some embodiments.

In an example, a hypothesis h may be provided and may be what is to be predicted. An instance v₁ . . . vₙ and corresponding defaults d₁ . . . dₙ, may be provided where the instance and default are not all the same. The variables may be ordered so that v₁ may be different from d₁. The following equality may be used:

$$\frac{P ( h \vert v 1 , v 2 … vn )}{P ( h \vert d 1 , d 2 … dn )} = \frac{P ( h \vert v 1 , v 2 … vn )}{P ( h \vert d 1 , v 2 … vn )} \frac{) P ( h \vert d 1 , v 2 … vn )}{P ( h \vert d 1 , d 2 … dn )}$$

On the right side of the equality, the denominator of the first fraction and the numerator of the second fraction are identical and cancel. The first fraction is of the form amenable to the ceteris paribus assumption, and this may be assumed as the same for all v₂ . . . vₙ. The second is of the same form as the term on the left of the equality, but has one fewer non-default values. This may be solved recursively with the same equation. This may be stopped with a value of 1 when the v's and d's are the same.

Rather than multiplying, it may be more natural to add, and in an example may be performed in the log space. The logarithms base 10 may be used, as these may be easier for people to interpret as orders of magnitude.

Given a feature Xᵢ and a value vᵢ (e.g., writing vᵢ for Xᵢ=vᵢ) and a default d that may specify a well-defined assignment of values to variables, and a hypothesis h, a reward may be defined as follows:

$${reward}_{d} ( h \vert {v}_{i} ) = {log}_{10} \frac{P ( h \vert {v}_{i} ∧ x )}{P ( h \vert {d}_{i} ∧ x )}$$

In the equation above, the ceteris paribus assumption may be that this may be the same for all x. The log may be base 10 so that the values may be interpreted as orders of magnitude. As further described below, the base may be omitted and may be assumed to be 10. It should be noted that although base 10 may be used herein, the embodiment anticipate using any base. Thus, the embodiments and corresponding example may be practiced using any base.

A model for a hypothesis may be a set of reward statements, with the assumption that propositions with no reward specified may have a reward of zero. A model for a hypothesis may specify how the prediction may differ from the default for one or more relevant features (e.g., each relevant feature). In examples, models may introduce new features, and these may be used without modifying other models. In example, most of the models may use a small subset of the features.

In an example, if soil slides were 10 times as likely on steep slopes as they are on moderate slopes, and moderate slopes were the default, we would have:

reward_{d}(soil slide|slope=steep)=1

reward_{d}(soil slide|slope=moderate)=0

where the second may not have been specified.

Taking logs turns product into sums. Sums may be easier to work with. The score of hypothesis h for instance v may be provide with respect to default d as:

$${scored}_{d} = log \frac{P ( h \vert v )}{P ( h \vert d )}$$

Under the ceteris paribus semantics for rewards, the scores may be the sum of rewards:

$${scored}_{d} ( h \vert {v}_{1} … {v}_{n} ) = {\sum }_{i} {reward}_{d} ( h \vert {v}_{i} )$$

The reward may be weaker than the probability. For example, knowing that a soil slide is 10 times as likely on a steep slope as a moderate slope, may not provide enough information to infer the probability of a soil slide. It may be inferred that the probability of a soil slide on a moderate slope is less than or equal to 0.1, because probability of a soil slide on a steep slope may be less than 1. The reward may not indicate the probability of a soil slide on other slopes.

The information to specify the scores may be strictly weaker than the probabilities may be provide. For example, given a reward for every non-default value of every variable, there are infinitely many probabilities that are consistent with the rewards. There may be two parts to this example. The first is that there may be at least one, and the second is that multiplying all of the probabilities by ε<1 may results in another consistent probability distribution.

If, the probabilities of the defaults are known, the probabilities may be computed as follows:

P(h|v)=P(h|d)*10^{score}^{_{d}}^{(h|v)} Equation (7)

This may specify how to transport the model to a new location. The probabilities may need to be calibrated by estimating P(h|d) for the new location. Because the default d may be fixed, one evaluation may be used for each hypothesis h for the new location, and predictions about the new location may be made combinatorially.

Instances may be compared. For example, multiple instances and a model may be used in a comparison. There may be multiple instances, and they may be compared to a model (e.g., a single model). For example, it may be desirable to know which location is more likely to have a soil slide, or which person is more likely to have a disease (and by how much). It might be more persuasive to claim that this location/person is 7.5 times as likely and another location/person to have a landslide/disease than to give an accurate assessment of a probability.

Given instance v:

score_{d}(h|v)=log P(h|v)−log P(h|d)]

Given another instance v′:

$${score}_{d} ( h \vert v ) - {score}_{d} ( h \vert {v}^{′} ) = log P ( h \vert v ) - log P ( h \vert {v}^{′} ) = log \frac{P ( h \vert v )}{P ( h \vert {v}^{′} )}$$

As the log probability of h given d cancels.

The difference in scores reflects may reflect ratio of the probability of the model given the instance, independently of the default. The difference in scores may be treated as a difference in probabilities, and although the scores may depend on the default, the difference in scores may not.

Model comparisons may be provided. For example, an instance may be compared to one or more models. In an example, there may be multiple models, and an instance (e.g., a single instance). For example, it may be desirable to know whether some location is more likely to have a soil slide or a rockfall, or whether someone is more likely to have covid-19 or the flu.

Given instance v and two models m₁ (about hypothesis h₁) and m₂ (about hypothesis h₂):

score_{d}(h₁|v)−score_{d}(h₂|v)=log P(h₁|v)−log P(h₁|d)−log P(h₂|v)+log P(h₂|d)

### Thus:

$$log \frac{P ( {h}_{1} \vert v )}{P ( {h}_{2} \vert v )} = {score}_{d} P ( {h}_{1} \vert v ) - {score}_{d} ( {h}_{2} \vert v ) + log \frac{P ( {h}_{1} \vert d )}{P ( {h}_{2} \vert d )}$$

The difference in scores may not be directly interpreted as how much more likely one hypothesis than another, but may need to be adjusted by

$$log \frac{P ( {h}_{1} \vert d )}{P ( {h}_{2} \vert d )} ,$$

which may be independent of the instance, and may reflect the relative probability of the hypotheses in the default situation.

Learning may be provided, such a learning for rewards. To learn the rewards, independence may be exploited. For example, if the ratio in the definition of reward is true for all x, then it may be true in expectation.

$$\begin{matrix}{reward}_{d} ( h \vert {v}_{i} ) = {log}_{10} \frac{P ( h \vert {v}_{i} ∧ x )}{P ( h \vert {d}_{i} ∧ x )} \\ = {log}_{10} \frac{P ( h \vert {v}_{i} )}{P ( h \vert {d}_{i} )}\end{matrix}$$

Because discrete values may be used, one variable may be addressed at a time, and it may be appropriate to assume a Dirichlet distribution, or a beta distribution for the Boolean case. A way to estimate the probability is in such models may be to use both the training data and pseudo-counts that reflect priors. For example:

$$p ( h \vert {v}_{i} ) = \frac{\# h ∧ {v}_{i} + {c}_{0}}{\# h ∧ {v}_{i} + \# ¬ h ∧ {v}_{i} + {c}_{1}}$$

where #h∧vᵢ may be the number of training examples for which h∧vᵢ is true, and #¬h∧vᵢ is the number of training examples for which vᵢ is true and h is false. c₀ and c₁ may be positive real numbers with c₁>c₀>0. The values c₀=1, c₁=2 may give Laplace smoothing, which may be appropriate if there is a uniform prior. The cᵢ dominate when there may be little data; when there are many examples, they may get washed out by the data.

The same may be done for P(h|dᵢ), and the ratio may be used as the reward. For simplicity, it may be assumed the same pseudo-counts may estimate the two probabilities. It may be a different number of examples that may be used to estimate P(h|vᵢ) and P(h|dᵢ).

If we write #vᵢ=#h∧vᵢ+#¬h∧vᵢ, which may be the number of times vᵢ is true and it may be known whether h may or may not be true. The following may be provided:

$${reward}_{d} ( h \vert {v}_{i} ) \approx log \frac{\# h ∧ {v}_{i} + {c}_{0}}{\# {v}_{i} + {c}_{1}} \times \frac{\# {d}_{i} + {c}_{1}}{\# h ∧ {d}_{i} + {c}_{0}} \qquad Equation ( 8 )$$

$$= log \frac{\# h ∧ {v}_{i} + {c}_{0}}{\# h ∧ {d}_{i} + {c}_{0}} \times \frac{\# {d}_{i} + {c}_{1}}{\# {v}_{i} + {c}_{1}} \qquad Equation ( 9 )$$

There may be positive evidence for h. For example, it may be known when h is true, it may not be known when it is false. For example, in the soil slides example described herein, there may be many examples of soil slides, but locations may not be interpreted without soil slides labelled as not having soil slides. However, in an example, positive examples may be used to estimate the left product of equation (9). The second product may be treated as the inverse of the proportion of vi compared to di in the population as a whole. This may not assume the closed world assumption, but that the same proportion of h may be missing when dᵢ and vᵢ true. More sophisticated solutions may be used when other models of missing data may be assumed.

In an example, other statistics that may be assessed for soil slides. For example, #vᵢ+c₁ may be assed (e.g., how many steep slopes are there). As another example, what proportion of the slopes are steep may be assessed. The ratio

$$\frac{\# h ∧ {v}_{i} + {c}_{0}}{\# {v}_{i} + {c}_{1}}$$

may be assessed, for example, to estimate what portion of the steep slopes have landslides, which may be very unstable as it may depend on the weather, the rocktype, and other factors.

The ratio

$$\frac{\# h ∧ {v}_{i} + {c}_{0}}{\# h ∧ {d}_{i} + {c}_{0}}$$

may be assessed, for example, to estimate how much more likely is a landslide on a steep slope compared to moderate slope. This ratio may be misleading as soil slides may be more common on moderate slopes than steep slopes, even though a steep slope may be more prone to soil slides, because moderate slopes may be more common. A value that may be used for the reward adjusts for this, and so may be applicable for areas with different proportions of slopes.

Recalibration may be provided. Recalibration may involve one or more changing defaults. In an example, a set of rewards may be calibrated to one default, and another set of rewards may be calibrated to another default. This may occur when the sets of rewards were designed by different people who happened to choose different defaults. The score and rewards may be recalibrated. For example, score or some rewards calibrated with respect d may be calibrated with respect to d′. The following may be used:

score_{d′}(h|v)=score_{d}(h|v)+score_{d′}(h|d)

reward_{d′}(hᵢ|vᵢ)=reward_{d}(h|vᵢ)+reward_{d′}(h|dᵢ)

Proof:

$$\frac{P ( h \vert v )}{P ( h \vert {d}^{′} )} = \frac{P ( h \vert v )}{P ( h \vert d )} \frac{P ( h \vert d )}{P ( h \vert {d}^{′} )}$$

where the P(h|d) cancel. Taking logs may provide the desired result for scores. The reward derivation may be similar. The scores may have one number for each h to recalibrate (e.g., score_{d′}(h|d)), but there may be one recalibration for each variable where they may differ for the rewards.

Interactions between features (e.g., conjunctions and other formulae) may be provided. In some examples, ceteris paribus may not be an appropriate assumption. Two values may be complements if both true gives more evidence than the sum of each individually. They may be substitutes if both true may give less evidence than the sum of each individually. For example, for landslides, high rainfall (e.g., a trigger) and loose soil (e.g., a propensity) may give a much higher probability of a landslide than either one alone, and may be considered complements. In a mountainous area on the west coast of a continent, facing west and having high rainfall both may provide a similar sort of information, and they may be considered substitutes.

To handle complements, substitutes, and more complex interactions, logical formulae may be used as parts of the rewards. For example, the following equation may provide a reward:

reward_{d}(soil_slide|slope=steep∧rain=high)=1.5

The reward above may specify that the probability of landslides may be increased when both the slope is steep, and the rainfall may be high. This may not give a reward when only one is true.

The definition of reward may be extended to allow for logical formulae on the right side of the |, which may provide the following:

$${score}_{d} ( h \vert v ) = {\sum }_{{reward}_{d} ( h \vert f ) \vert v \vert = f} {reward}_{d} ( h \vert f ) \qquad Equation ( 10 )$$

where v|=f means that f may be true of the assignment v. This may be summing over one or more rewards statement in the model (e.g., all of the reward statements in the model) for which the formula is true of the instance v.

In an example, the reward may be provided by the following:

reward_{d}(soil_slide|Facing=west∨Rain=high)=0.8

This may indicate an increase of probability of 10^{0.8}≈6.3 compared to the default if either the rainfall is high, or the slope faces West. This disjunction may be equivalent to:

reward_{d}(soil_slide|Facing=west)=0.8

reward_{d}(soil_slide|Rain=high)=0.8

reward_{d}(soil_slide|Facing=west∧Rain=high)=−0.8

where the last equation may not allow for double score counting when both rainfall is high and the slope faces West.

The reward may be provided as the logarithm of the ratio of probabilities. The reward may be the value that make Equation (10) hold. For example, the reward of conjunctions may be as follows, which may occur even in the presence of rewards for atomic propositions:

$$\begin{matrix}{reward}_{d} ( h \vert {v}_{1} ∧ {v}_{2} ) = log \frac{P ( h \vert {v}_{1} ∧ {v}_{2} )}{P ( h \vert {v}_{1} ∧ {d}_{2} )} \frac{P ( h \vert {d}_{1} ∧ {d}_{2} )}{P ( h \vert {d}_{1} ∧ {v}_{2} )} \\ = \begin{matrix}log P ( h \vert {v}_{1} ∧ {v}_{2} ) + log P ( h \vert {d}_{1} ∧ {d}_{2} ) - \\ log P ( h \vert {v}_{1} ∧ {d}_{2} ) - log P ( h \vert {d}_{1} ∧ {v}_{2} )\end{matrix}\end{matrix} \qquad Equation ( 11 )$$

The left product of Equation (11) may indicate how much the probability may change going from d₂ to v₂ in the presence of v₁. The left product of Equation (11) may indicate an inverse of how much the probability changes going from d₂ to v₂ in the presence of d₁. When the ceteris paribus may hold, how much the probability changes going from d₂ to v₂ may be the same whether v₁ or d₁ holds, and so product may be one and the logarithm may be zero.

The diagnosticity may be transferable from one domain to another. For example, conditionals may be learned (e.g., conditional probabilities or rewards/scores) in British Columbia (BC), Canada and they may be applied and/or tested in another location, such as in Veneto, Italy. The two location may have different distributions of slopes, clearcuts and landslides.

The prediction of P(y|x1 . . . xₙ) may be evaluated for multiple instances of Y and xᵢ using both log-likelihood and sum-of-squares error. This may be tested for y being slope slide and rock fall, and the xᵢ being slopes, rocktype, clearcut, and the like. A number of comparisons may be performed. In an example, the probability in may be learned BC and may be applied in Veneto with and without Laplace smoothing (e.g., adding a pseudo count of 1). In an example, a logistic regression model in BC may be learned and may be applied in Veneto. In an example, rewards in BC may be learned, and then scores may be predicted in Veneto, which may involve adjusting the default for Veneto using Equation (7).

Diagnosticity may be an approach to provide a preference score between one or more entities based on probability (e.g., a frequency) of attributes and their importance (e.g., a diagnosticity). This may be used to search and rank entities in a database. For example, diagnosticity may be used to assist in searching for an apartment, a product, and the like. There may be a number of approaches to diagnosticity. For example, there may be a default model diagnosticity approach and a default instance diagnosticity approach.

The default model diagnosticity approach may be used when data is missing (for example, when silence may not imply absence) and the missing data may be inferred from global probability distributions. The default model diagnosticity approach may be used when data is not missing (e.g., when silence may imply absence).

A default model diagnosticity approach may be provided. For example, diagnosticity scores may be used that may be based on a global default: scores may be determined by comparing preference on instance values (e.g., a default model) to the global probability distribution of instance attributes. This may be useful when precise attribute values may be hard to quantify precisely (e.g., they may be missing or may not be specified), and it may be easier to quantify their probability. Probability distributions of attribute values may be quantified based on data or expert judgment. For example, a geologist may not know if there is gold in a specific land area, but she may guess the probability of the presence of gold given the global distribution of gold in rocks or on her expert judgment of presence of gold on that specific region of the world.

The default model diagnosticity approach may be used in a number of fields. For example, the default model diagnosticity approach may be used to provide product recommendations, apartment reconditions, medical recommendations, geological recommendations, and the like. A default model diagnosticity approach may be used to provide apartment recommendations, which may be based on user preferences. For example, a family may be moving to Vancouver from the United States for work. A house model (e.g., an ideal house model) may be created for the family. For example, a real estate agent may create a model based on her expertise in understanding what the family may be seeking. An example of the house model may be seen Table 4. The house model may be used to query the available apartment database. Apartments may be ranked by similarity to the model by adding the diagnosticity scores of one or more attributes (e.g., each attribute).

TABLE 4
Model of an ideal apartment for a family

| | | | Probability | | |
|---|---|---|---|---|---|
| | | | in the | | |
| | Attribute | Probability in | background | Diagnosticity | Diagnosticity |
| Attribute | value | the model | (default) | if present | if absent |
| Has room | 3 | 0.99 (People | 0.5 (half of the | 0.297 | −1.699 |
| | | want 3 | available | | |
| | | bedrooms: one | houses have | | |
| | | for them and one | 3 bedrooms) | | |
| | | room for each of | | | |
| | | their 2.2 | | | |
| | | statistical kids) | | | |
| Has distance | 10 | 0.8 (People want | 0.6 | 0.204 | −0.398 |
| to school | minutes | a short commute | | | |
| | | but it may not be | | | |
| | | as important as | | | |
| | | the number of | | | |
| | | rooms) | | | |
| Has | Not | 0.01 (people may | 0.9 (most | −1.954 | 0.996 |
| neighborhood | crime | not want an | available | | |
| safety | | unsafe | houses may | | |
| | | neighborhood) | be in | | |
| | | | neighborhoods | | |
| | | | that are less | | |
| | | | safe) | | |

In this example, the apartment recommendation may take into consideration the realtor's knowledge of the world (e.g., peoples' preferences for apartments) which may be expressed as probabilities between 0 and 1 in the “probability in the model” field. The apartment recommendation may take into consideration the probability distribution of attributes values between 0 and 1 in the “probability in the background (default)” field, which may be obtained from an apartment database and/or the realtor domain expertise. The score may be expressed as the logarithm of the ratio between “probability in the model” field and “probability in the in the background (default)” field, as shown in Table 4. By adding the scores of one or more attributes (e.g., each attribute,) a total score may be obtained that may allow for ranking of the apartment instances.

A default instance diagnosticity approach may be provided. The default instance diagnosticity approach may use diagnosticity scores that may be based on a local default. For example, scores may be determined by comparing instance values to the values of a known (e.g., default) instance. This approach may be useful when it may be hard to define global probability distributions of attributes, and instead local probabilities may be compared. Local probabilities may be based on data (e.g., this person may be 7.5 times more likely to get that disease than this other person) or on subjective preferences (e.g., this person values the quality of a neighborhood of a house twice as much as the house age).

The default instance diagnosticity approach may be used in a number of fields. For example, the default model diagnosticity approach may be used to provide product recommendations, apartment reconditions, medical recommendations, geological recommendations, and the like.

A default instance diagnosticity approach may be used to provide apartment recommendations. For example, a real estate agent may be interviewing an international student that has just moved to Canada. The student has been assigned to an old one-bedroom apartment in East Van but she is not happy with it and she asks for other options.

The real estate agent may have two other options: a new one-bedroom apartment in one neighborhood in Squamish and an old two-bedroom apartment in a second neighborhood in Squamish. The real estate agent may wish to understand which one of the two apartments the student may like the most as compared to the default apartment in East Van that the student has been assigned to. The real estate agent may interview the student to determine the student's preference. The default instance diagnosticity approach may compare the preferences to the available apartments and provide a recommendation.

For example, the student's preferences may indicate that the student doesn't like that the apartment in East Van is old, near a major street, and far from hiking trails. The student's preference may indicate that the student likes that the apartment in East Van is near stores and in a young neighborhood. The student's preference may indicate that the student would like a newer apartment, with more space (2 bedroom), in a young neighborhood, with a nice view of the mountains. The student's preferences may indicate that the student would like an apartment near stores and hiking. The student's preference may indicate that the student would prefer not to spend more than $2,000 for two bedroom ($1,000 per room) or $1,500 for one bedroom.

The student preferences may be input into the default instance diagnosticity approach. The student preferences may be adjusted with positive and negative rewards for an apartment attribute (e.g., each apartment attribute). For example, a reward of +1 may be given to the age attribute of the apartment (e.g., apartment has age: new=+1). The scale ranges for the reward may be between −1 and +1. Zero may be a default for the scale range. The scale range may be logarithmic, such that that 1 may be 10 times more than 0. The awards may be adjusted programmatically, by a user, or a combination of both. For example, the real estate agent may adjust a score for a price per room based on feedback from the student.

Table 5 shows an instance for the apartment in East Van, which may be used as a default instance with the default instance diagnosticity approach.

TABLE 5
Default: old one-bedroom in East Van

| Attribute | Attribute value | Score |
|---|---|---|
| Has room | 1 | — |
| Has age | old | — |
| Has price per room | $1,200 | — |
| Has view | Street | — |
| Has noise | High | — |
| Has neighborhood | young professional | — |
| demographic | | |
| Has distance to store | 20 min | — |
| Has distance to hiking | 60 min | — |
| | | — |

Table 6 shows an instance for a first apartment in Squamish, which may be used as an instance with the default instance diagnosticity approach. The first apartment may be a new one-bedroom apartment that may be in a young neighborhood.

TABLE 6
A new one-bedroom in Squamish, young neighborhood.

| Attribute | Attribute value | Score |
|---|---|---|
| Has room | 1 | 0 |
| Has age | New | +1 |
| Has price per room | $1,700 | −0.5 |
| Has view | Mountain | +1 |
| Has noise | quiet | +1 |
| Has neighborhood | Young professional | 0 |
| demographic | | |
| Has distance to store | 40 min | −0.9 |
| Has distance to hiking | 10 min | +1 |
| | | Total = +2.6 |

Table 7 shows an instance for a second apartment in Squamish, which may be used as an instance with the default instance diagnosticity approach. The second apartment may be an old two-bedroom apartment that may be in an old neighborhood.

TABLE 7
An old two-bedroom in Squamish, old neighborhood

| Attribute | Attribute value | Score |
|---|---|---|
| Has room | 2 | +1 |
| Has age | Old | 0 |
| Has price per room | $850 | +0.8 |
| Has view | Street | 0 |
| Has noise | quiet | +1 |
| Has neighborhood | Old retired | −1 |
| demographic | | |
| Has distance to store | 40 min | −0.9 |

The default instance diagnosticity approach may indicate that the student has a preference for the first apartment. For example, the default instance diagnosticity approach may indicate to the real estate agent that the student may like either of the apartments in Squamish more than the one in East Van, with a preference for the new one-bedroom apartment.

The apartment recommendation based on Tables 5-7 may be based on personal preferences expressed as probabilities between −1 and +1 on a logarithmic scale. Attributes may be determined based on available information and the default may be arbitrary. For example, it may be the apartment the student has been assigned to. In another example, the default may be another apartment, such as the first apartment, or the second apartment. By adding the scores of one or more attributes (e.g., each attribute), a total score may be obtained that may allow for the apartment instances to be ranked based on client's preferences.

A device may be provided for expressing a diagnosticity of an attribute in a conceptual model. The device may comprise a memory and a processor. The processor may be configured to perform a number of actions. One or more model attributes that may be relevant for a model may be determined. The model may be defined. For example, the model may be defined by expressing for a model attribute (e.g., each model attribute) at least two of a frequency of the model attribute in the model, a frequency of the model attribute in a default model, a diagnosticity of a presence of the model attribute, and a diagnosticity of an absence of the model attribute.

An instance that may comprising one or more instance attributes may be determined. An instance attribute in the one or more instance attributes may be assigned a positive diagnosticity when the instance attribute may be present. An instance attribute in the one or more instance attributes may be assigned a negative diagnosticity when the instance attribute may be absent (e.g., may not be present).

A predictive score for the instance may be determined. For example, the predictive score for the instance may be determined by summing one or more contributions made by the one or more instance attributes.

An explanation associated with the predictive score may be determined for the one or more attributes using one or more of the frequency of the model attribute in the model, and the frequency of the model attribute in the default model. For example, an explanation associated with the predictive score may be determined for each model attribute in the one or more model attributes using the frequency of the model attribute in the model and the frequency of the model attribute in the default model.

In an example, the predictive score may indicate a predictability or likeliness of the model.

In an example the instance may be a first instance, the predictive score may be a first predictive score. A second instance may be determined. A second predictive score may be determined. A comparative score may be determined. For example, a comparative score may be determined using the first predictive score and the second predictive score, the comparative score indicating whether the first instance or the second instance offers a better prediction.

In an example, the positive diagnosticity may be associated with a diagnosticity of the presence of a correlating model attribute from the one or more model attributes. In an example, the negative diagnosticity may be associated with a diagnosticity of the absence of a correlating model attribute from the one or more model attributes.

In an example, a prior score of the model may be determined. For example, a prior score of the model may be determined by comparing a probability of the model to a default model.

In an example, a posterior score for the model and the instance may be determined. For example, a posterior score for the model and the instance may be determined using the prior score and the predictive score.

A device may be provided for expressing a probabilistic reasoning of an attribute in a conceptual model. The device may comprise a memory and a processor. The processor may be configured to perform a number of actions. A model attribute that may be relevant for a model may be determined. The model may be determined by expressing at least two of a frequency of the model attribute in the model, a frequency of the model attribute in a default model, a probabilistic reasoning of a presence of the model attribute, a probabilistic reasoning of an absence of the model attribute. An instance may be determined. The instance may comprise at least an instance attribute that may have a positive probabilistic reasoning or a negative probabilistic reasoning. A predictive score for the instance may be determined. For example, a predictive score for the instance may be determined using a contribution made by the instance attribute. An explanation associated with the predictive score may be determined. For example, an explanation associated with the predictive score may be determined using the frequency of the model attribute in the model and the frequency of the model attribute in the default model.

In an example, the instance may be a first instance and the predictive score may be a first predictive score. A second instance may be determined. A second predictive score may be determined. A comparative score may be determined. For example, a comparative score may be determined using the first predictive score and the second predictive score. The comparative score may indicate whether the first instance or the second instance offers a better prediction.

In an example, the predictive score may indicate a predictability or likeliness of the model.

In an example, the positive probabilistic reasoning may be associated with the probabilistic reasoning of the presence of the model attribute. In an example, the negative probabilistic reasoning may be associated with the probabilistic reasoning of the absence of the model attribute.

In an example, a prior score of the model may be determined. For example, a prior score of the model may be determined by comparing a probability of the model to a default model.

In an example, a posterior score for the model and the instance may be determined. For example, a posterior score for the model and the instance may be determined using the prior score and the predictive score.

A method may be provided that may be performed by a device for expressing a probabilistic reasoning of an attribute in a conceptual model. A model attribute that is relevant for a model may be determined. The model may be determined. For example, the model may be determined by expressing at least two of a frequency of the model attribute in the model, a frequency of the model attribute in a default model, a probabilistic reasoning of a presence of the model attribute, a probabilistic reasoning of an absence of the model attribute. An instance may be determined. The instance may comprise at least an instance attribute that may have a positive probabilistic reasoning or a negative probabilistic reasoning. A predictive score may be determined for the instance. For example, the predictive score may be determined using a contribution made by the instance attribute. An explanation associated with the predictive score may be determined. For example, an explanation may be determined using the frequency of the model attribute in the model and the frequency of the model attribute in the default model.

In an example, the predictive score may indicate a predictability or likeliness of the model.

In an example, the positive probabilistic reasoning may be associated with the probabilistic reasoning of a presence of the model attribute. In an example, the negative probabilistic reasoning may be associated with the probabilistic reasoning of the absence of the model attribute.

In an example, a prior score of the model may be determined. For example, a prior score of the model may be determined by comparing a probability of the model to a default model.

In an example, a posterior score for the model and the instance may be determined. For example, a posterior score for the model and the instance may be determined using the prior score and the predictive score.

It will be appreciated that while illustrative embodiments have been disclosed, the scope of potential embodiments is not limited to those explicitly described. For example, while may probabilistic reasoning being applied to geology, mineral discovery, and/or apartment searching, probabilistic reasoning may be applied to other domains of expertise. For example, probabilistic reasoning may be applied to computer security, healthcare, real estate, land using planning, insurance markets, medicine, finance, law, and/or the like.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable storage media include, but are not limited to, a read only memory (ROM), a random access memory (RAM), a register, cache memory, semiconductor memory devices, magnetic media such as internal hard disks and removable disks, magneto-optical media, and optical media such as CD-ROM disks, and digital versatile disks (DVDs).

What is claimed:

1. A device for expressing a diagnosticity of an attribute in a conceptual model, the device comprising:
a memory, and
a processor, the processor configured to:
determine one or more model attributes that are relevant for a model;
define the model by expressing, for each model attribute in the one or more model attributes, at least two of a frequency of the model attribute in the model, a frequency of the model attribute in a default model, a diagnosticity of a presence of the model attribute, and a diagnosticity of an absence of the model attribute;
determine an instance comprising one or more instance attributes, wherein an instance attribute in the one or more instance attributes is assigned a positive diagnosticity when the instance attribute is present and is assigned a negative diagnosticity when the instance attribute is absent;
determine a predictive score for the instance by summing contributions made by the one or more instance attributes; and
determine an explanation associated with the predictive score for each model attribute in the one or more model attributes using the frequency of the model attribute in the model and the frequency of the model attribute in the default model.

2. The device of claim 1, wherein the predictive score indicates a predictability or likeliness of the model.

3. The device of claim 1, wherein the instance is a first instance, the predictive score is a first predictive score, and the processor is further configured to:
determine a second instance;
determine a second predictive score; and
determine a comparative score using the first predictive score and the second predictive score, the comparative score indicating whether the first instance or the second instance offers a better prediction.

4. The device of claim 1, wherein the positive diagnosticity is associated with a diagnosticity of the presence of a correlating model attribute from the one or more model attributes.

5. The device of claim 1, wherein the negative diagnosticity is associated with a diagnosticity of the absence of a correlating model attribute from the one or more model attributes.

6. The device of claim 1, wherein the processor is further configured to determine a prior score of the model by comparing a probability of the model to a default model.

7. The device of claim 6, wherein the processor is further configured to determine a posterior score for the model and the instance using the prior score and the predictive score.

8. A device for expressing a probabilistic reasoning of an attribute in a conceptual model, the device comprising:
a memory, and
a processor, the processor configured to:
determine a model attribute that is relevant for a model;
determine the model by expressing at least two of a frequency of the model attribute in the model, a frequency of the model attribute in a default model, a probabilistic reasoning of a presence of the model attribute, a probabilistic reasoning of an absence of the model attribute;
determine an instance comprising at least an instance attribute that has a positive probabilistic reasoning or a negative probabilistic reasoning;
determine a predictive score for the instance using a contribution made by the instance attribute; and
determine an explanation associated with the predictive score using the frequency of the model attribute in the model and the frequency of the model attribute in the default model.

9. The device of claim 8, wherein the instance is a first instance, the predictive score is a first predictive score, and the processor is further configured to:
determine a second instance;
determine a second predictive score; and
determine a comparative score using the first predictive score and the second predictive score, the comparative score indicating whether the first instance or the second instance offers a better prediction.

10. The device of claim 8, wherein the predictive score indicates a predictability or likeliness of the model.

11. The device of claim 8, wherein the positive probabilistic reasoning is associated with the probabilistic reasoning of the presence of the model attribute.

12. The device of claim 8, wherein the negative probabilistic reasoning is associated with the probabilistic reasoning of the absence of the model attribute.

13. The device of claim 8, wherein the processor is further configured to determine a prior score of the model by comparing a probability of the model to a default model.

14. The device of claim 13, wherein the processor is further configured to determine a posterior score for the model and the instance using the prior score and the predictive score.

15. A method performed by a device for expressing a probabilistic reasoning of an attribute in a conceptual model, the method comprising:
determining a model attribute that is relevant for a model;
determining the model by expressing at least two of a frequency of the model attribute in the model, a frequency of the model attribute in a default model, a probabilistic reasoning of a presence of the model attribute, a probabilistic reasoning of an absence of the model attribute;
determining an instance comprising at least an instance attribute that has a positive probabilistic reasoning or a negative probabilistic reasoning;
determining a predictive score for the instance using a contribution made by the instance attribute; and
determining an explanation associated with the predictive score using the frequency of the model attribute in the model and the frequency of the model attribute in the default model.

16. The method of claim 15, wherein the predictive score indicates a predictability or likeliness of the model.

17. The method of claim 15, wherein the positive probabilistic reasoning is associated with the probabilistic reasoning of a presence of the model attribute.

18. The method of claim 15, wherein the negative probabilistic reasoning is associated with the probabilistic reasoning of the absence of the model attribute.

19. The method of claim 15, further comprising determining a prior score of the model by comparing a probability of the model to a default model.

20. The method of claim 19, further comprising determining a posterior score for the model and the instance using the prior score and the predictive score.
